(19) United States
(12) Patent Application Publication
(10) Pub. No.: US 2025/0305001 A1
(43) Pub. Date: Oct. 2, 2025

(54) Genetic Construct

(71) Applicants: Healing Genes LLC, New York, NY (US); Panacea Venture Healthcare Fund I, LP, Shanghai (CN)

(72) Inventor: Michael McDonald, Guildford (GB)

(73) Assignees: Healing Genes LLC, New York, NY (US); Panacea Venture Healthcare Fund I, LP, Shanghai (CN)

(21) Appl. No.: 19/203,701

(22) Filed: May 9, 2025

Related U.S. Application Data
(63) Continuation of application No. 16/616,302, filed on Nov. 22, 2019, now Pat. No. 12,319,927.

(30) Foreign Application Priority Data
May 25, 2017 (GB) 1708385.8

(51) Int. Cl. C12N 15/86; A61K 48/00; A61P 25/28; C12N 9/02; C12N 9/78

(52) U.S. Cl. CPC C12N 15/86; A61P 25/28; C12N 9/0071; C12N 9/78; C12Y 114/16002; C12Y 305/04016; A61K 48/00; C12N 2750/14132; C12N 2750/14143

(57) ABSTRACT

A genetic construct comprises a promoter operably linked to a first coding sequence, which encodes tyrosine hydroxylase (TH), and a second coding sequence, which encodes GTP cyclohydrolase 1 (GCH1), wherein the second coding sequence is 3′ to the first coding sequence, and the first and second coding sequences are part of a single operon. The genetic construct does not encode aromatic amino acid decarboxylase (AADC).

The present invention relates to genetic constructs and recombinant vectors comprising such constructs, and to the uses of the constructs and vectors in gene therapy methods for treating neurodegenerative disorders, such as Parkinson's disease.

Parkinson's disease is a neurodegenerative disease associated with the loss of dopamine-producing cells in the striatum. There are three enzymes which are necessary for the production of dopamine by brain cells: tyrosine hydroxylase (TH), GTP cyclohydrolase 1 (GCH1) and aromatic amino acid decarboxylase (AADC). TH and GCH1 regulate the production of L-DOPA (a precursor to dopamine) from tyrosine, and AADC converts L-DOPA to dopamine. The current treatment options for Parkinson's disease include oral administration of L-DOPA, which, in contrast to dopamine, is absorbed across the blood-brain barrier. This treatment is efficacious because AADC is still present in the brains of Parkinson's disease patients.

Oral L-DOPA therapy can lead to side effects, such as abnormal movement. These side effects are believed to be due to the fluctuation levels of L-DOPA in blood and brain caused by the short half-life of L-DOPA and the variable absorption across the gut mucosa and blood brain barrier resulting from competition with other amino for active transport (Lees, April 2008, The Importance of Steady-State plasma DOPA levels in reducing motor fluctuations in Parkinson's disease, Expert Roundtable Supplement, CNS Spectr 13:4 (Suppl 7) P4-7).

Gene therapy for Parkinson's disease involves the transfer of a vector into the striatum, where the vector carries genes necessary for the production of dopamine or L-DOPA by brain cells that would ordinarily be non-dopamine producing. The aim of such treatment is the local generation of dopamine within the affected areas of the brains of Parkinson's patients. Several methods of gene therapy have been disclosed. However, while the technique has shown promise, and the previous methods provide a proof of the principle, previous vectors have not been optimal. In particular, there is a need for vectors that lead to optimal production of dopamine (either directly or indirectly via L-DOPA) in the brains of Parkinson's patients, and which can be manufactured at suitable levels and with suitable cost effectiveness to be a viable treatment option.

Muramatsu et al. (10 Feb. 2002, Behavioral Recovery in a Primate Model of Parkinson's disease by Tripe Transduction of Striatal Cells with Adeno-Associated Viral (AAV) Vectors Expressing dopamine-Synthesizing Enzymes, Human Gene Therapy, 12:345-354) conducted the first study to show complete recovery on a primate model of Parkinson's disease by transfer of the genes for TH, GCH and AADC into the striatum. This was achieved by administering three types of vector, one for each of TH, GCH1 and AADC. This approach had two significant issues: (1) the ratio of genes transfected to any particular neuron is random, and (2) the cost involved in manufacturing and releasing three separate vectors is prohibitive. As a result, the approach was never advanced to a clinical product. Expressing all three genes within a single AAV vector was not possible because the size of the genes exceeded what could be accommodated within the vector.

To overcome this limitation, a lentiviral vector construct was disclosed for use in treating Parkinson's disease (WO2013/061076 and WO2010/055209). This used a much larger lentiviral vector to accommodate all three genes, TH, GCH1 and AADC within a single vector (Jarraya, et al., 14 Oct. 2009, Dopamine Gene Therapy for Parkinson's Disease in a Nonhuman Primate Without Associated Dyskinesia, Science Translational Medicine, Vol 1 Issue 2). This showed promising results in non-human primates, but when tested in a clinical trial, the magnitude of efficacy reported was only within the placebo range reported in other clinical trials for Parkinson's disease using surgical techniques (Palfi et al., 29 Mar. 2014, Long-term safety and tolerability of ProSavin, a lentiviral vector-based gene therapy for Parkinson's disease: a dose escalation, open-label, phase 1/2 trial, The Lancet, Vol 383). The limited efficacy observed, and failure in producing, and dose, lentivector at sufficient titres resulted in a decision by the originators to discontinue clinical development of the product.

As L-DOPA administered orally or intravenously was known to be effective in reversing the motor symptoms of Parkinson's disease it was apparent that Parkinson's disease patients retained sufficient AADC activity in the brain to convert L-DOPA to dopamine. Cederfjall E. et al therefore developed a single AAV vector co expressing TH and GCH1 (WO2011/054976 and WO2015152813). This construct was effective in completely reversing motor symptoms in the rat 6-OHDA model of Parkinson's disease but failed in a non-human primate model. Post-mortem assessments of transgene expression in the treated macaques demonstrated robust expression of GCH1, and GFP controls, but not TH. This was in contrast to the finding in rodents using the same vector preparation where robust expression of both TH and GCH1 was observed. Cederfjall et al subsequently wrote “The reason for the lack of transgenic TH expression by histology and lack of DOPA and dopamine production by microdialysis remains unclear at this time. However, this problem requires a solution prior to the initiation of clinical trials utilizing this approach.” (Cederfjall E et al July 2013 Continuous DOPA synthesis from a single AAV: dosing and efficacy in models of Parkinson's disease, Scientific Reports, Vol 3).

Segawa syndrome is a rare (orphan) indication due to mutations of the guanosine triphosphate cyclohydrolase I (GCH-1) gene. The GCH-1 gene mutation is inherited as an autosomal dominant trait or occurs as a spontaneous genetic change (i.e., new mutation). Due to the rareness of Segawa syndrome it may not be commercially attractive or viable to develop a treatment for this indication.

There is therefore a need for improved constructs suitable for use in treating neurodegenerative diseases, in particular diseases associated with catecholamine dysfunction, for instance Parkinson's disease.

The inventor has constructed a novel genetic construct, which leads to improved production of GCH1 and TH, and hence is suitable for use with an improved method of treatment for neurodegenerative diseases, in particular diseases associated with catecholamine dysfunction, such as Parkinson's disease.

Thus, according to a first aspect of the invention, there is provided a genetic construct comprising a promoter operably linked to a first coding sequence, which encodes tyrosine hydroxylase (TH), and a second coding sequence, which encodes GTP cyclohydrolase 1 (GCH1), wherein the second coding sequence is 3′ to the first coding sequence, and the first and second coding sequences are part of a single operon, and wherein the genetic construct does not encode aromatic amino acid decarboxylase (AADC).

The genetic construct of the first aspect, which comprises TH and GCH1, but which does not include AADC, is advantageous for several reasons. Firstly, the construct guarantees that the two genes, TH and GCH1, are delivered to the same cells in a subject being treated. Furthermore, the costs and difficulties associated with the production of multiple vectors, as would be required if the genes were present in different constructs, as in the prior art, are avoided. The inclusion of TH and GCH1 without AADC is advantageous because the delivery of these two polypeptides is sufficient for a therapeutic effect (Kirik D, et al., Reversal of motor impairments in parkinsonian rats by continuous intrastriatal delivery of L-DOPA using rAAV-mediated gene transfer. PNAS, 2002, 99, 4708-13), and allows a more efficacious and easier to manufacture smaller vector. For instance, the size limitation of rAAV vectors will prevent the incorporation of large gene constructs, reducing the production titres and efficacy.

WO2013/061076 and WO2010/055209 disclose expression constructs encoding TH, GCH1 and AADC, and their use in Parkinson's disease, and both documents regard all three proteins as being essential to provide a therapeutic effect. Furthermore, both documents are silent on the advantages of using a single promoter approach, as provided in the genetic construct of the first aspect. Thus, these documents teach that, TH, GCH1 and AADC are required to produce a therapeutic effect, and that fusions of these proteins may be particularly effective. However, these documents are silent on the advantages or disadvantages of different promoter set-ups. This is important, as single constructs comprising all three proteins TH, GCH1 and AADC have insufficient efficacy, and it is not possible to get a high enough titre to produce a therapeutic effect.

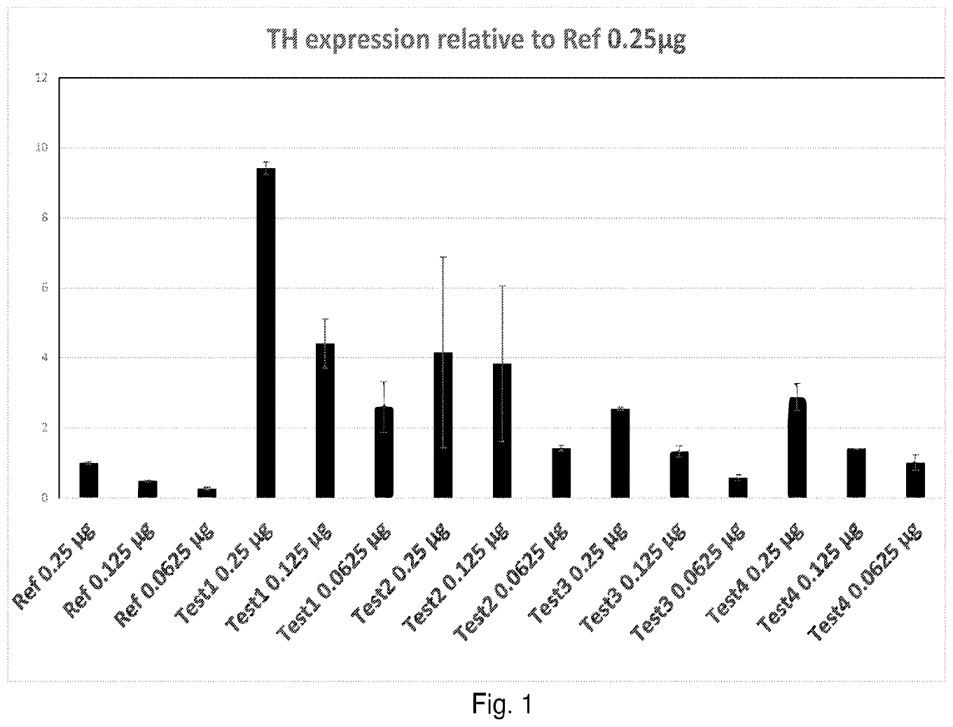

WO2011/054976 and WO2015152813 disclose an AAV vector which comprises GCH1 driven by a promoter, and TH driven by a separate promoter. However, as shown in FIG. 1, the inventor of the construct of the first aspect has tested the construct described in WO2011/054976, and clearly demonstrated that it does not achieve optimal levels of TH or GCH-1 expression, and is therefore not an optimal construct for use in gene therapy. Before the data presented herein, the skilled person would have been unaware of the sub-optimal expression of the construct of WO2011/054976. While variants of this construct were disclosed in WO2011/054976, the data are provided for the dual promoter construct, and the disclosure cautioned against variants which would be likely to have unpredictable, and hence negative, effects.

The term “operon” can mean a group of linked genes that produce a single messenger RNA molecule during transcription. Thus, the first coding sequence and the second coding sequence are under the control of the same promoter. The construct does not include a separate promoter for each cistron.

It is particularly important during gene therapy for a genetic construct to lead to high levels of the encoded polypeptides, because greater production of TH and GCH1 can lead to a greater therapeutic effect. However, small variations between constructs can lead to large and unpredictable differences in gene expression. For instance, Hennecke et al. state “These observations led us to conclude that IRES-dependent translation is not predictable and obviously depends on the composition of the mRNA” (Hennecke et al. Composition and arrangement of genes define the strength of IRES-driven translation in bicistronic mRNAs. Nucleic Acids Research, 2001, Vol. 29, No. 16, P3327-3334). Furthermore, the theoretical maximal level of polypeptide production for TH and GCH1 is not known; new constructs are tested by examining the relative expression of the genes in comparison to controls. Hence, it is only possible with hindsight to recognise non-optimal vectors. Without wishing to be bound to any particular theory, the inventor has surprisingly found that a separate promoter approach, as taught in the prior art, results in problematic interference between the promoters, such that the expression of one or both genes is reduced to sub-therapeutic levels.

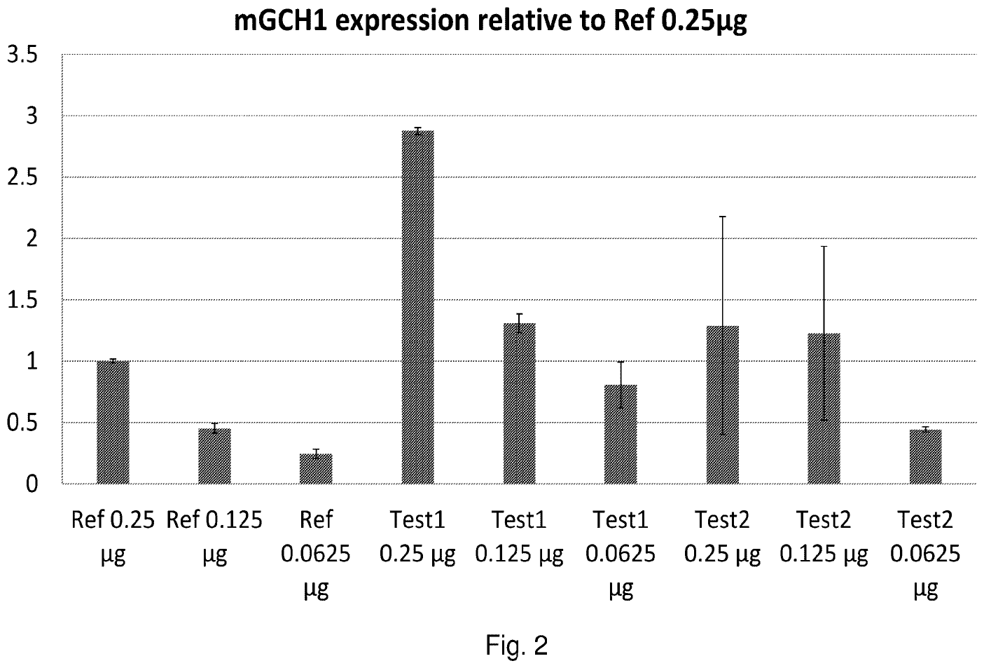

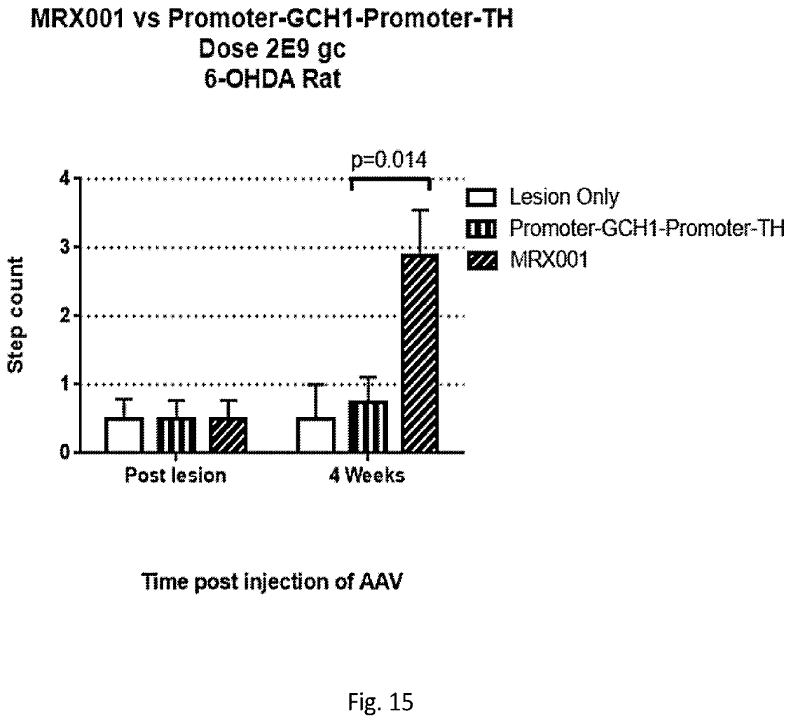

The inventor presents data herein that show that the disclosed genetic constructs lead to improved production of TH and GCH1 in comparison to the prior art constructs (FIG. 1, FIG. 2, Table 1, and Table 2). It is only with hindsight, based upon this new data, that the inventor has identified problems with the prior art constructs. Furthermore, the inventor has shown, in vivo, that improved production of the disclosed genetic constructs leads to a significantly increased efficacy in a Parkinson's disease rat model in comparison to the prior art constructs (FIG. 15).

It is particularly surprising that the construct of the first aspect leads to improved expression over the co-administration of the monocistronic constructs, and this could not have been predicted. The technical prejudice of the skilled person is that monocistronic constructs lead to the highest level of expression, and that bicistronic constructs would not be as effective. However, the new data disclosed herein, for the first time indicate that constructs which correspond to embodiments of the first aspect (Test 1, Test 2, and Test 3) actually lead to surprisingly higher mRNA expression than the co-administered monocistronic constructs (Test4). The full details of the experiments and the resultant data are disclosed in the Examples section.

Furthermore, the construct of the first aspect leads to improved expression the bicistronic construct of WO 2011/054976 A2. The reference construct described in the Examples corresponds to the construct disclosed by WO 2011/054976 A2. As can be seen, the present genetic constructs have surprisingly improved TH and GCH1 expression. The full details of the experiments and the resultant data are disclosed in the Examples section.

It is especially surprising that the constructs of the invention lead to improved expression of a 3′ GCH1 over both the prior art bicistronic construct and over co-administration of the two monocistronic constructs. In general, it is expected that the 3′ gene in a bicistronic construct may be expressed at a lower level than if the gene were 5′. Accordingly, it is very surprising that the construct with a 3′ GCH1 is able to produce improved levels of GCH1, even over constructs comprising a 5′ GCH1.

In an embodiment, the relative expression of TH is 1.1 to 100 fold higher than the expression of TH from a reference construct comprising a first promoter sequence operably linked to a sequence encoding GCH1 and a second promoter sequence operably linked to a sequence encoding TH. In an embodiment, the relative expression is 1.5 to 20 fold higher, 2 to 15 fold higher, or between 3 and 10 fold higher. In an embodiment, the reference construct is the construct described as the reference construct in the Examples. In an embodiment, the in vitro assay to determine the level of expression is the in vitro assay disclosed in the Examples. In an embodiment, the relative expression is assayed using 0.25 μg of plasmid for transfection. In an embodiment, the relative expression is assayed using 0.0625 μg of plasmid for transfection.

In an embodiment, the relative expression of GCH1 is 1.1 to 10 fold higher than the expression of GCH1 from a reference construct comprising a first promoter sequence operably linked to a sequence encoding GCH1 and a second promoter sequence operably linked to a sequence encoding TH. In an embodiment, the relative expression is 1.2 to 5 fold higher, or between 2 to 4 fold higher. In an embodiment, the reference construct is the construct described as the reference construct in the Examples. In an embodiment, the in vitro assay to determine the level of expression is the in vitro assay disclosed in the Examples. In an embodiment, the relative expression is assayed using 0.25 μg of plasmid for transfection. In an embodiment, the relative expression is assayed using 0.0625 μg of plasmid for transfection.

In one embodiment, the first coding sequence comprises a nucleotide sequence encoding human TH. The nucleotide sequence encoding human TH is referred to herein as SEQ ID No:1, or a fragment or variant thereof, as set out below:

[SEQ ID NO: 1]
atgcccacccccgacgccaccacgccacaggccaagggcttccgcagg
gccgtgtctgagctggacgccaagcaggcagaggccatcatgtccccg
cggttcattgggcgcaggcagagcctcatcgaggacgcccgcaaggag
cgggaggcggcggtggcagcagcggccgctgcagtcccctcggagccc
ggggaccccctggaggctgtggcctttgaggagaaggaggggaaggcc
gtgctaaacctgctcttctccccgagggccaccaagccctcggcgctg
tcccgagctgtgaaggtgtttgagacgtttgaagccaaaatccaccat
ctagagacccggcccgcccagaggccgcgagctgggggcccccacctg
gagtacttcgtgcgcctcgaggtgcgccgaggggacctggccgccctg
ctcagtggtgtgcgccaggtgtcagaggacgtgcgcagccccgcgggg
cccaaggtcccctggttcccaagaaaagtgtcagagctggacaagtgt
catcacctggtcaccaagttcgaccctgacctggacttggaccacccg
ggcttctcggaccaggtgtaccgccagcgcaggaagctgattgctgag
atcgccttccagtacaggcacggcgacccgattccccgtgtggagtac
accgccgaggagattgccacctggaaggaggtctacaccacgctgaag
ggcctctacgccacgcacgcctgcggggagcacctggaggcctttgct
ttgctggagcgcttcagcggctaccgggaagacaatatcccccagctg
gaggacgtctcccgcttcctgaaggagcgcacgggcttccagctgcgg
cctgtggccggcctgctgtccgcccgggacttcctggccagcctggcc
ttccgcgtgttccagtgcacccagtatatccgccacgcgtcctcgccc
atgcactcccctgagccggactgctgccacgagctgctggggcacgtg
cccatgctggccgaccgcaccttcgcgcagttctcgcaggacattggc
ctggcgtccctgggggcctcggatgaggaaattgagaagctgtccacg
ctgtactggttcacggtggagttcgggctgtgtaagcagaacggggag
gtgaaggcctatggtgccgggctgctgtcctcctacggggagctcctg
cactgcctgtctgaggagcctgagattcgggccttcgaccctgaggct
gcggccgtgcagccctaccaagaccagacgtaccagtcagtctacttc
gtgtctgagagcttcagtgacgccaaggacaagctcaggagctatgcc
tcacgcatccagcgccccttctccgtgaagttcgacccgtacacgctg
gccatcgacgtgctggacagcccccaggccgtgcggcgctccctggag
ggtgtccaggatgagctggacacccttgcccatgcgctgagtgccatt
ggctag

Preferably, therefore, the first coding sequence comprises a nucleotide sequence substantially as set out in SEQ ID No:1, or a fragment or variant thereof.

In one preferred embodiment, the first coding sequence comprises a nucleotide sequence encoding human TH. Human TH may have an amino acid sequence according to NCBI Reference Sequence: NP_000351.2, which is referred to herein as SEQ ID NO: 21, or a fragment or variant thereof, as set out below:

[SEQ ID NO: 21]
MPTPDATTPQAKGFRRAVSELDAKQAEAIMSPRFIGRRQSLIEDARKER
EAAVAAAAAAVPSEPGDPLEAVAFEEKEGKAVLNLLFSPRATKPSALSR
AVKVFETFEAKIHHLETRPAQRPRAGGPHLEYFVRLEVRRGDLAALLSG
VRQVSEDVRSPAGPKVPWFPRKVSELDKCHHLVTKFDPDLDLDHPGFSD
QVYRQRRKLIAEIAFQYRHGDPIPRVEYTAEEIATWKEVYTTLKGLYAT
HACGEHLEAFALLERFSGYREDNIPQLEDVSRFLKERTGFQLRPVAGLL
SARDFLASLAFRVFQCTQYIRHASSPMHSPEPDCCHELLGHVPMLADRT
FAQFSQDIGLASLGASDEEIEKLSTLYWFTVEFGLCKQNGEVKAYGAGL
LSSYGELLHCLSEEPEIRAFDPEAAAVQPYQDQTYQSVYFVSESFSDAK
DKLRSYASRIQRPFSVKFDPYTLAIDVLDSPQAVRRSLEGVQDELDTLA
HALSAIG*

Preferably, therefore, the first coding sequence comprises a nucleotide sequence encoding an amino acid sequence substantially as set out in SEQ ID No:21, or a fragment or variant thereof.

In another embodiment, the first coding sequence comprises a nucleotide sequence encoding human truncated TH. Human truncated TH is a variant of TH with only the catalytic domain, and with the regulatory domain removed. The domains of TH and their roles are described in Daubner et al. (Daubner S C, Lohse D L, Fitzpatrick’ PF. Expression and characterization of catalytic and regulatory domains of rat tyrosine hydroxylase. Protein Sci. 1993; 2:1452-60). Human truncated TH comprises the nucleotide sequence referred to herein as SEQ ID No:2, or a fragment or variant thereof, as set out below:

[SEQ ID NO: 2]
atgagccccgcggggcccaaggtcccctggttcccaagaaaagtgtcag
agctggacaagtgtcatcacctggtcaccaagttcgaccctgacctgga
cttggaccacccgggcttctcggaccaggtgtaccgccagcgcaggaag
ctgattgctgagatcgccttccagtacaggcacggcgacccgattcccc
gtgtggagtacaccgccgaggagattgccacctggaaggaggtctacac
cacgctgaagggcctctacgccacgcacgcctgcggggagcacctggag
gcctttgctttgctggagcgcttcagcggctaccgggaagacaatatcc
cccagctggaggacgtctcccgcttcctgaaggagcgcacgggcttcca
gctgcggcctgtggccggcctgctgtccgcccgggacttcctggccagc
ctggccttccgcgtgttccagtgcacccagtatatccgccacgcgtcct
cgcccatgcactcccctgagccggactgctgccacgagctgctggggca
cgtgcccatgctggccgaccgcaccttcgcgcagttctcgcaggacatt
ggcctggcgtccctgggggcctcggatgaggaaattgagaagctgtcca
cgctgtactggttcacggtggagttcgggctgtgtaagcagaacgggga
ggtgaaggcctatggtgccgggctgctgtcctcctacggggagctcctg
cactgcctgtctgaggagcctgagattcgggccttcgaccctgaggctg
cggccgtgcagccctaccaagaccagacgtaccagtcagtctacttcgt
gtctgagagcttcagtgacgccaaggacaagctcaggagctatgcctca
cgcatccagcgccccttctccgtgaagttcgacccgtacacgctggcca
tcgacgtgctggacagcccccaggccgtgcggcgctccctggagggtgt
ccaggatgagctggacacccttgcccatgcgctgagtgccattggctag

Preferably, therefore, the first coding sequence comprises a nucleotide sequence substantially as set out in SEQ ID No:2, or a fragment or variant thereof.

In one preferred embodiment, the first coding sequence comprises a nucleotide sequence encoding human truncated TH. Human truncated TH comprises an amino acid sequence referred to herein as SEQ ID NO: 22, or a fragment or variant thereof, as set out below:

[SEQ ID NO: 22]
MSPAGPKVPWFPRKVSELDKCHHLVTKFDPDLDLDHPGFSDQVYRQRRK
LIAEIAFQYRHGDPIPRVEYTAEEIATWKEVYTTLKGLYATHACGEHLE
AFALLERFSGYREDNIPQLEDVSRFLKERTGFQLRPVAGLLSARDFLAS
LAFRVFQCTQYIRHASSPMHSPEPDCCHELLGHVPMLADRTFAQFSQDI
GLASLGASDEEIEKLSTLYWFTVEFGLCKQNGEVKAYGAGLLSSYGELL
HCLSEEPEIRAFDPEAAAVQPYQDQTYQSVYFVSESFSDAKDKLRSYAS
RIQRPFSVKFDPYTLAIDVLDSPQAVRRSLEGVQDELDTLAHALSAIG*

Preferably, therefore, the first coding sequence comprises a nucleotide sequence encoding an amino acid sequence substantially as set out in SEQ ID No:22, or a fragment or variant thereof.

In an embodiment, the second coding sequence comprises a nucleotide sequence encoding murine GCH1. The nucleotide sequence encoding murine GCH1 is referred to herein as SEQ ID No:3, or a fragment or variant thereof:

[SEQ ID NO: 3]
ggtggttttcctttgaaaaacacgatgataatatggccacaaccgcggc
cgtagatcccgggaccatggagaagccgcggggagtcaggtgcaccaat
gggttctccgagcgggagctgccgcggcccggggccagcccgcctgccg
agaagtcccggccgcccgaggccaagggcgcacagccggccgacgcctg
gaaggcagggcggcaccgcagcgaggaggaaaaccaggtgaacctcccc
aaactggcggctgcttactcgtccattctgctctcgctgggcgaggacc
cccagcggcaggggctgctcaagacgccctggagggcggccaccgccat
gcagtacttcaccaagggataccaggagaccatctcagatgtcctgaat
gatgctatatttgatgaagatcatgacgagatggtgattgtgaaggaca
tagatatgttctccatgtgtgagcatcaccttgttccatttgtaggaag
ggtccatattggctatcttcctaacaagcaagtccttggtctcagtaaa
cttgccaggattgtagaaatctacagtagacgactacaagttcaagagc
gcctcaccaaacagattgcggtggccatcacagaagccttgcagcctgc
tggcgttggagtagtgattgaagcgacacacatgtgcatggtaatgcga
ggcgtgcagaaaatgaacagcaagactgtcactagcaccatgctgggcg
tgttccgggaagaccccaagactcgggaggagttcctcacactaatcag
gagctgag

Therefore, the second coding sequence may comprise a nucleotide sequence substantially as set out in SEQ ID No:3, or a fragment or variant thereof.

In a preferred embodiment, the second coding sequence comprises a nucleotide sequence encoding human GCH1. For example, the sequence encoding human GCH may be the sequence according to GenBank NM 000161.2. The nucleotide sequence encoding human GCH1 is referred to herein as SEQ ID No:4, or a fragment or variant thereof, as set out below:

[SEQ ID NO: 4]
atggagaagggccctgtgcgggcaccggcggagaagccgcggggcgcca
ggtgcagcaatgggttccccgagcgggatccgccgcggcccgggcccag
caggccggcggagaagcccccgcggcccgaggccaagagcgcgcagccc
gcggacggctggaagggcgagcggccccgcagcgaggaggataacgagc
tgaacctccctaacctggcagccgcctactcgtccatcctgagctcgct
gggcgagaacccccagcggcaagggctgctcaagacgccctggagggcg
gcctcggccatgcagttcttcaccaagggctaccaggagaccatctcag
atgtcctaaacgatgctatatttgatgaagatcatgatgagatggtgat
tgtgaaggacatagacatgttttccatgtgtgagcatcacttggttcca
tttgttggaaaggtccatattggttatcttcctaacaagcaagtccttg
gcctcagcaaacttgcgaggattgtagaaatctatagtagaagactaca
agttcaggagcgccttacaaaacaaattgctgtagcaatcacggaagcc
ttgcggcctgctggagtcggggtagtggttgaagcaacacacatgtgta
tggtaatgcgaggtgtacagaaaatgaacagcaaaactgtgaccagcac
aatgttgggtgtgttccgggaggatccaaagactcgggaagagttcctg
actctcattaggagctga

Preferably, therefore, the second coding sequence comprises a nucleotide sequence substantially as set out in SEQ ID No: 4, or a fragment or variant thereof.

In one preferred embodiment, the second coding sequence comprises a nucleotide sequence encoding human GCH1. Human GCH1 may have an amino acid sequence according to NCBI Reference Sequence: NP_000152.1. Human GCH1 comprises an amino acid sequence referred to herein as SEQ ID NO: 23, or a fragment or variant thereof, as set out below:

[SEQ ID NO: 23]
MEKGPVRAPAEKPRGARCSNGFPERDPPRPGPSRPAEKPPRPEAKSAQP
ADGWKGERPRSEEDNELNLPNLAAAYSSILSSLGENPQRQGLLKTPWRA
ASAMQFFTKGYQETISDVLNDAIFDEDHDEMVIVKDIDMFSMCEHHLVP
FVGKVHIGYLPNKQVLGLSKLARIVEIYSRRLQVQERLTKQIAVAITEA
LRPAGVGVVVEATHMCMVMRGVQKMNSKTVTSTMLGVFREDPKTREEFL
TLIRS*

Preferably, therefore, the second coding sequence comprises a nucleotide sequence encoding an amino acid sequence substantially as set out in SEQ ID No:23, or a fragment or variant thereof.

The genetic construct according to the first aspect comprises a promoter. The promoter may be any suitable promoter, including a constitutive promoter, an activatable promoter, an inducible promoter, or a tissue-specific promoter. In a preferred embodiment, the promoter is a one enabling the generation of TH and GCH1 in the most suitable tissue or tissues for therapy. In an embodiment, the promoter is one that permits high expression in neurons, such as for example striatal neurons. The promoter may be a neuron-specific promoter.

In an embodiment, the promoter is the CMV promoter, one embodiment of which is referred to herein as SEQ ID NO: 25, as follows:

[SEQ ID No: 25]
ACGCGTGGAGCTAGTTATTAATAGTAATCAATTACGGGGTCATTAGTTC
ATAGCCCATATATGGAGTTCCGCGTTACATAACTTACGGTAAATGGCCC
GCCTGGCTGACCGCCCAACGACCCCCGCCCATTGACGTCAATAATGACG
TATGTTCCCATAGTAACGTCAATAGGGACTTTCCATTGACGTCAATGGG
TGGAGTATTTACGGTAAACTGCCCACTTGGCAGTACATCAAGTGTATCA
TATGCCAAGTACGCCCCCTATTGACGTCAATGACGGTAAATGGCCCGCC
TGGCATTATGCCCAGTACATGACCTTATGGGACTTTCCTACTTGGCAGT
ACATCTACGTATTAGTCATCGCTATTACCATGGTGATGCGGTTTTGGCA
GTACATCAATGGGCGTGGATAGCGGTTTGACTCACGGGGATTTCCAAGT
CTCCACCCCATTGACGTCAATGGGAGTTTGTTTTGCACCAAAATCAACG
GGACTTTCCAAAATGTCGTAACAACTCCGCCCCATTGACGCAAATGGGC
GGTAGGCGTGTACGGTGGGAGGTCTATATAAGCAGAGCTCGTTTAGTGA
ACCGTCAGATCGCCTGGAGACGCCATCCACGCTGTTTTGACCTCCATAG
AAGACACCGGGACCGATCCAGCCTCC

In an embodiment, the promoter may be a human synapsin promoter. In an embodiment, the promoter is a human synapsin 1 promoter. One embodiment of the 469 nucleotide sequence encoding the human synapsin I (SYN I) promoter is referred to herein as SEQ ID NO: 5, as follows:

[SEQ ID NO: 5]
CTGCAGAGGGCCCTGCGTATGAGTGCAAGTGGGTTTTAGGACCAG
GATGAGGCGGGGTGGGGGTGCCTACCTGACGACCGACCCCGACCC
ACTGGACAAGCACCCAACCCCCATTCCCCAAATTGCGCATCCCCT
ATCAGAGAGGGGGAGGGGAAACAGGATGCGGCGAGGCGCGTGCGC
ACTGCCAGCTTCAGCACCGCGGACAGTGCCTTCGCCCCCGCCTGG
CGGCGCGCGCCACCGCCGCCTCAGCACTGAAGGCGCGCTGACGTC
ACTCGCCGGTCCCCCGCAAACTCCCCTTCCCGGCCACCTTGGTCG
CGTCCGCGCCGCCGCCGGCCCAGCCGGACCGCACCACGCGAGGCG
CGAGATAGGGGGGCACGGGCGCGACCATCTGCGCTGCGGCGCCGG
CGACTCAGCGCTGCCTCAGTCTGCGGTGGGCAGCGGAGGAGTCGT
GTCGTGCCTGAGAGCGCAG

Preferably, therefore, the promoter may comprise a nucleotide sequence substantially as set out in SEQ ID No: 5 or 25, or a fragment or variant thereof.

The genetic construct may further comprise one or more enhancer, which is configured to increase the expression of TH or GCH1. In particular, the construct may comprise an enhancer designed to cooperate with the promoter. As an example, a construct including a CMV promoter may also include a CMV enhancer.

In a preferred embodiment, the genetic construct comprises a spacer sequence disposed between the first and second coding sequences. This spacer sequence is such that it allows the production of functional TH and the production of functional GCH1 from the single promoter. In an embodiment, the spacer sequence comprises a sequence that allows for translation initiation in the middle of an mRNA sequence as part of the greater process of protein synthesis. In a particularly preferred embodiment, the spacer sequence comprises an internal ribosome entry site (IRES). The data presented herein clearly demonstrate that a construct including a 5′ TH and a 3′ GCH1 separated by an IRES leads to a surprisingly effective genetic construct (FIG. 1 and FIG. 2). In an embodiment, the IRES is a picornavirus IRES.

In other embodiments, the IRES may be selected from a rhinovirus IRES, a hepatitis A virus IRES, a hepatitis C virus IRES, a poliovirus IRES, an enterovirus IRES, a cardiovirus IRES, an aphthovirus IRES, flavivirus IRES, a pestivirus IRES, a cripavirus IRES, a rhopalosiphum padi virus IRES, or any suitable IRES. In particular, the IRES may be any IRES described by the “IRESite” which provides a database of experimentally verified IRES structures (http://www.iresite.org/), or as disclosed in “New Messenger RNA Research Communications” (ISBN: 1-60021-488-6).

In a preferred embodiment, the IRES is a foot-and-mouth disease virus (FMDV) IRES, which may be as set out in SEQ ID No:6, or a fragment or variant thereof, as follows:

[SEQ ID NO: 6]
AGCAGGTTTCCCCAACTGACACAAAACGTGCAACTTGAAACTCCG
CCTGGTCTTTCCAGGTCTAGAGGGGTAACACTTTGTACTGCGTTT
GGCTCCACGCTCGATCCACTGGCGAGTGTTAGTAACAGCACTGTT
GCTTCGTAGCGGAGCATGACGGCCGTGGGAACTCCTCCTTGGTAA
CAAGGACCCACGGGGCCAAAAGCCACGCCCACACGGGCCCGTCAT
GTGTGCAACCCCAGCACGGCGACTTTACTGCGAAACCCACTTTAA
AGTGACATTGAAACTGGTACCCACACACTGGTGACAGGCTAAGGA
TGCCCTTCAGGTACCCCGAGGTAACACGCGACACTCGGGATCTGA
GAAGGGGACTGGGGCTTCTATAAAAGCGCTCGGTTTAAAAAGCTT
CTATGCCTGAATAGGTGACCGGAGGTCGGCACCTTTCCTTTGCAA
TTACTGACCAC

In another preferred embodiment, the IRES is an encephalomyocarditis virus (EMCV) IRES. The EMCV IRES may be as set out in SEQ ID No:7, or a fragment or variant thereof, as follows:

[SEQ ID NO: 7]
cgttactggccgaagccgcttggaataaggccggtgtgcgtttgtctat
atgttattttccaccatattgccgtcttttggcaatgtgagggcccgga
aacctggccctgtcttcttgacgagcattcctaggggtctttcccctct
cgccaaaggaatgcaaggtctgttgaatgtcgtgaaggaagcagttcct
ctggaagcttcttgaagacaaacaacgtctgtagcgaccctttgcaggc
agcggaaccccccacctggcgacaggtgcctctgcggccaaaagccacg
tgtataagatacacctgcaaaggcggcacaaccccagtgccacgttgtg
agttggatagttgtggaaagagtcaaatggctcccctcaagcgtattca
acaaggggctgaaggatgcccagaaggtaccccattgtatgggatctga
tctggggcctcggtgcacatgcttttcatgtgtttagtcgaggttaaaa
aacgtctaggccccccgaaccacggggacgtggttttcctttgaaaaac
acgatgataata

Therefore, preferably the IRES comprises a nucleotide sequence substantially as set out in SEQ ID No: 6 or 7, or a fragment or variant thereof.

Alternatively, instead of an IRES, the spacer sequence may comprise a nucleotide sequence encoding a peptide spacer that is configured to be digested to thereby produce the TH and GCH1 as separate molecules. Preferably, the spacer sequence comprises and encodes a viral peptide spacer sequence, more preferably a viral 2A peptide spacer sequence (Furler S, Paterna J-C, Weibel M and Bueler H Recombinant AAV vectors containing the foot and mouth disease virus 2A sequence confer efficient bicistronic gene expression in cultured cells and rat substantia nigra neurons Gene Ther. 2001, vol. 8, PP: 864-873). Preferably, the spacer sequence encoding the 2A peptide sequence connects the first coding sequence to the second coding sequence. This enables the construct to overcome the size restrictions that occur with expression in various vectors and enables expression of all of the peptides encoded by the construct of the first aspect to occur under control of a single promoter, as a single protein. Thus, following the translation of the single protein comprising the sequences of TH, the 2A peptide, and GCH1, cleavage occurs in the viral 2A peptide sequence at the terminal glycine-proline link, thereby liberating two proteins. The data presented herein demonstrate that a construct including a 5′ TH and a 3′ GCH1 separated by a viral 2A peptide spacer sequence leads to a surprisingly effective genetic construct (FIG. 1 and FIG. 2).

In an embodiment, the spacer comprises a viral 2A peptide spacer and further comprises a furin cleavage site. Insertion of an upstream furin cleavage site allows the removal of 2A residues that would otherwise remain attached to the upstream protein.

In an embodiment, the nucleotide sequence of a peptide spacer encoding both a viral 2A sequence and a furin cleavage site may be referred to herein as SEQ ID No:8, or a fragment or variant thereof, as follows:

[SEQ ID NO: 8]
cgcgcgaaacgcgcgccggtgaaacagaccctgaactttgatctgctga
aactggcgggcgatgtggaaagcaacccgggcccg

Preferably, therefore, the spacer sequence comprises a nucleotide sequence substantially as set out in SEQ ID No: 8, or a fragment or variant thereof.

The 2A spacer sequence may be any known variant, which includes those sequences referred to as E2A, F2A, P2A and T2A, as disclosed in Wang Y et al. Scientific Reports 2015, 5.

In one embodiment, the sequence is E2A, referred to herein as SEQ ID No: 27, as follows:

[SEQ ID NO: 27]
CAGTGTACTAATTATGCTCTCTTGAAATTGGCTGGAGATGTTGAGAGCA
ACCCTGGACCT

Preferably, therefore, the spacer sequence comprises a nucleotide sequence substantially as set out in SEQ ID No: 27, or a fragment or variant thereof.

In one embodiment, the sequence is F2A, referred to herein as SEQ ID No: 28, as follows:

[SEQ ID NO: 28]
GTGAAACAGACTTTGAATTTTGACCTTCTCAAGTTGGCGGGAGACGTGG
AGTCCAACCCTGGACCT

Preferably, therefore, the spacer sequence comprises a nucleotide sequence substantially as set out in SEQ ID No: 28, or a fragment or variant thereof.

In one embodiment, the sequence is P2A, referred to herein as SEQ ID No: 29, as follows:

[SEQ ID NO: 29]
GCCACGAACTTCTCTCTGTTAAAGCAAGCAGGAGATGTTGAAGAAAACC
CCGGGCCT

Preferably, therefore, the spacer sequence comprises a nucleotide sequence substantially as set out in SEQ ID No: 29, or a fragment or variant thereof.

In one embodiment, the sequence is T2A, referred to herein as SEQ ID No: 30, as follows:

[SEQ ID NO: 30]
GAGGGCAGGGGAAGTCTTCTAACATGCGGGGACGTGGAGGAAAATCCCG
GCCCC

Preferably, therefore, the spacer sequence comprises a nucleotide sequence substantially as set out in SEQ ID No: 30, or a fragment or variant thereof.

In an embodiment, the 2A sequence may be preceded by any sequence that improves the efficiency of 2A, i.e. the sequence is positioned 5′ to the 2A sequence. In an embodiment, the sequence that improves the efficiency of 2A is a glycine-serine-glycine spacer (GSG), referred to herein as SEQ ID No: 31, as follows:

[SEQ ID NO: 31]
GGAAGCGGA

Preferably, the 2A sequence is preceded by a nucleotide sequence substantially as set out in SEQ ID No: 31, or a fragment or variant thereof.

Alternatively, instead of an IRES or a viral 2A spacer, the spacer sequence may comprise a sequence encoding a flexible linker, which allows for the expression of both TH and GCH1 as a single polypeptide chain, but wherein the TH and GCH1 act as independent proteins. Hence, the proteins exert their effects in the same manner as if they were singly expressed. The data presented herein demonstrate that a construct including a 5′ TH and a 3′ GCH1 separated by spacer sequence comprising a flexible linker sequence leads to a surprisingly effective genetic construct (FIG. 1).

The flexible linker sequence may be as disclosed by WO 2013/061076 A1 (Oxford Biomedica), where this known linker was included in a tricistronic construct. The flexible linker sequence may be referred to herein as SEQ ID No:9, or a fragment or variant thereof, as follows:

[SEQ ID NO: 9]
ggaggtggcgggtccgggggcgggggtagcggtggcgggggctcc

Preferably, therefore, the flexible linker sequence comprises a nucleotide sequence substantially as set out in SEQ ID No: 9, or a fragment or variant thereof.

In one preferred embodiment, the flexible linker sequence comprises a nucleotide sequence encoding an amino acid sequence referred to herein as SEQ ID NO: 24, or a fragment or variant thereof, as set out below:

[SEQ ID NO: 24]
GGGGSGGGGSGGGGS

Preferably, therefore, the flexible linker sequence encodes an amino acid sequence substantially as set out in SEQ ID No: 24, or a fragment or variant thereof.

In an embodiment, the genetic construct may further comprise a nucleotide sequence encoding Woodchuck Hepatitis Virus Post-transcriptional Regulatory Element

(WPRE), which enhances the expression of the two transgenes. Preferably, the WPRE coding sequence is disposed 3′ of the transgene coding sequence. In particular, the WPRE sequence is preferably 3′ of the GCH1 sequence.

One embodiment of the WPRE is 592 bp long, including gamma-alpha-beta elements, and is referred to herein as SEQ ID No: 10, as follows:

[SEQ ID NO: 10]
AATCAACCTCTGGATTACAAAATTTGTGAAAGATTGACTGGTATTCTTA
ACTATGTTGCTCCTTTTACGCTATGTGGATACGCTGCTTTAATGCCTTT
GTATCATGCTATTGCTTCCCGTATGGCTTTCATTTTCTCCTCCTTGTAT
AAATCCTGGTTGCTGTCTCTTTATGAGGAGTTGTGGCCCGTTGTCAGGC
AACGTGGCGTGGTGTGCACTGTGTTTGCTGACGCAACCCCCACTGGTTG
GGGCATTGCCACCACCTGTCAGCTCCTTTCCGGGACTTTCGCTTTCCCC
CTCCCTATTGCCACGGCGGAACTCATCGCCGCCTGCCTTGCCCGCTGCT
GGACAGGGGCTCGGCTGTTGGGCACTGACAATTCCGTGGTGTTGTCGGG
GAAGCTGACGTCCTTTCCATGGCTGCTCGCCTGTGTTGCCACCTGGATT
CTGCGCGGGACGTCCTTCTGCTACGTCCCTTCGGCCCTCAATCCAGCGG
ACCTTCCTTCCCGCGGCCTGCTGCCGGCTCTGCGGCCTCTTCCGCGTCT
TCGCCTTCGCCCTCAGACGAGTCGGATCTCCCTTTGGGCCGCCTCCCCG
CCTG

Preferably, the WPRE comprises a nucleic acid sequence substantially as set out in SEQ ID No: 10, or a fragment or variant thereof.

However, in a preferred embodiment, a truncated WPRE is used, which is 247 bp long due to deletion of the beta element, and which is referred to herein as SEQ ID No: 11, as follows:

[SEQ ID NO: 11]
AATCAACCTCTGGATTACAAAATTTGTGAAAGATTGACTGGTATTCTTA
ACTATGTTGCTCCTTTTACGCTATGTGGATACGCTGCTTTAATGCCTTT
GTATCATGCTATTGCTTCCCGTATGGCTTTCATTTTCTCCTCCTTGTAT
AAATCCTGGTTAGTTCTTGCCACGGCGGAACTCATCGCCGCCTGCCTTG
CCCGCTGCTGGACAGGGGCTCGGCTGTTGGGCACTGACAATTCCGTGGT
GT

Preferably, the WPRE comprises a nucleic acid sequence substantially as set out in SEQ ID No: 11, or a fragment or variant thereof.

Preferably, the genetic construct comprises a nucleotide sequence encoding a polyA tail. Preferably, the polyA tail coding sequence is disposed 3′ of the transgene coding sequence, and preferably 3′ of the WHPE coding sequence.

Preferably, the polyA tail comprises the simian virus 40 poly-A 224 bp sequence. One embodiment of the polyA tail is referred to herein as SEQ ID No: 12, as follows:

[SEQ ID NO: 12]
AGCAGACATGATAAGATACATTGATGAGTTTGGACAAACCACAACTAGA
ATGCAGTGAAAAAAATGCTTTATTTGTGAAATTTGTGATGCTATTGCTT
TATTTGTAACCATTATAAGCTGCAATAAACAAGTTAACAACAACAATTG
CATTCATTTTATGTTTCAGGTTCAGGGGGAGGTGTGGGAGGTTTTTTAA
AGCAAGTAAAACCTCTACAAATGTGGTA

Preferably, the polyA tail comprises a nucleic acid sequence substantially as set out in SEQ ID No: 12, or a fragment or variant thereof.

Preferably, the genetic construct comprises left and/or right Inverted Terminal Repeat sequences (ITRs). Preferably, each ITR is disposed at the 5′ and/or 3′ end of the construct.

In a preferred embodiment, the genetic construct comprises, in this specified order, a 5′ human synapsin 1 promoter or a CMV promoter; a sequence encoding human truncated TH; an IRES; and a 3′ sequence encoding human GCH1. The use of 5′ and 3′ indicates that the features are either upstream or downstream, and is not intended to indicate that the features are necessarily terminal features.

In a preferred embodiment, the genetic construct may comprise, in this specified order, a 5′ ITR; a human synapsin 1 promoter or a CMV promoter; a sequence encoding human truncated TH; an IRES; a sequence encoding human GCH1; a sequence encoding WPRE; a sequence encoding a poly A tail; and a 3′ ITR.

In a particular embodiment the genetic construct may comprise, in this specified order, 5′ ITR; a human synapsin 1 promoter or a CMV promoter; a sequence encoding human truncated TH; a Furin-2A sequence; a sequence encoding human GCH1; a sequence encoding WPRE; a sequence encoding a poly A tail; and a 3′ ITR.

In a particular embodiment the genetic construct may comprise, in this specified order, a 5′ ITR; a human synapsin 1 promoter or a CMV promoter; a sequence encoding human truncated TH; a flexible linker; a sequence encoding human GCH1; a sequence encoding WPRE; a sequence encoding a poly A tail; and a 3′ ITR.

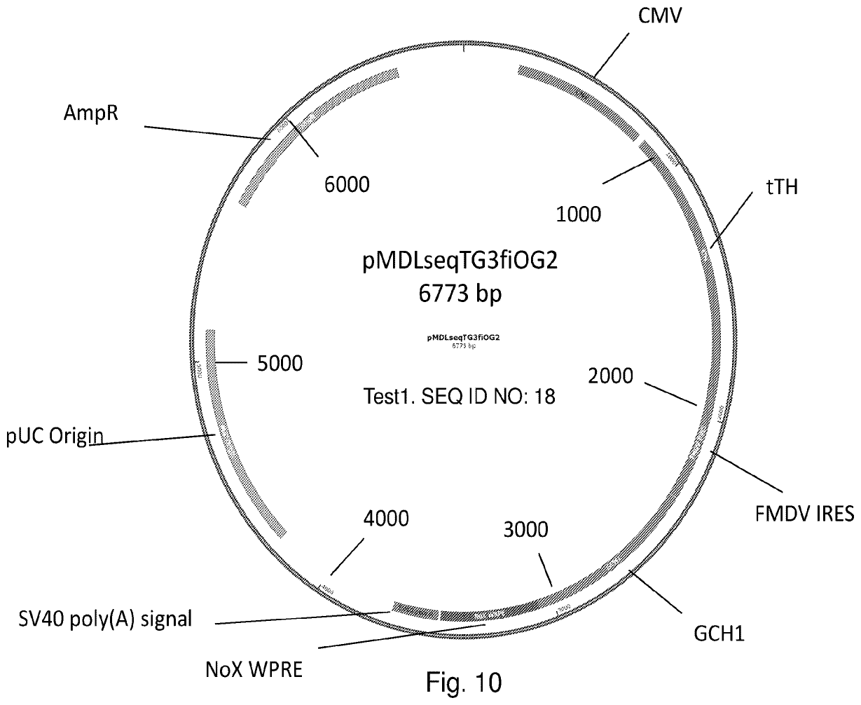

One embodiment of the genetic construct is shown in FIG. 10, and is referred to herein as SEQ ID No: 18. This particular embodiment includes a CMV promoter and murine GCH1; these features would be easily replaceable by the skilled person for other variants as disclosed herein.

[SEQ ID NO: 18]
ggcgatcgcggctcccgacatcttggaccattagctccacaggtatctt
cttccctctagtggtcataacagcagcttcagctacctctcaattcaaa
aaacccctcaagacccgtttagaggccccaaggggttatgctatcaatc
gttgcgttacacacacaaaaaaccaacacacatccatcttcgatggata
gcgattttattatctaactgctgatcgagtgtagccagatctagtaatc
aattacggggtcattagttcatagcccatatatggagttccgcgttaca
taacttacggtaaatggcccgcctggctgaccgcccaacgacccccgcc
cattgacgtcaataatgacgtatgttcccatagtaacgccaatagggac
tttccattgacgtcaatgggtggagtatttacggtaaactgcccacttg
gcagtacatcaagtgtatcatatgccaagtacgccccctattgacgtca
atgacggtaaatggcccgcctggcattatgcccagtacatgaccttatg
ggactttcctacttggcagtacatctacgtattagtcatcgctattacc
atgctgatgcggttttggcagtacatcaatgggcgtggatagcggtttg
actcacggggatttccaagtctccaccccattgacgtcaatgggagttt
gttttggcaccaaaatcaacgggactttccaaaatgtcgtaacaactcc
gccccattgacgcaaatgggcggtaggcgtgtacggtgggaggtctata
taagcagagctggtttagtgaaccgtcagatcagatctagagatcccgg
gaccgccaccatgagccccgcggggcccaaggtcccctggttcccaaga
aaagtgtcagagctggacaagtgtcatcacctggtcaccaagttcgacc
ctgacctggacttggaccacccgggcttctcggaccaggtgtaccgcca
gcgcaggaagctgattgctgagatcgccttccagtacaggcacggcgac
ccgattccccgtgtggagtacaccgccgaggagattgccacctggaagg
aggtctacaccacgctgaagggcctctacgccacgcacgcctgcgggga
gcacctggaggcctttgctttgctggagcgcttcagcggctaccgggaa
gacaatatcccccagctggaggacgtctcccgcttcctgaaggagcgca
cgggcttccagctgcggcctgtggccggcctgctgtccgcccgggactt
cctggccagcctggccttccgcgtgttccagtgcacccagtatatccgc
cacgcgtcctcgcccatgcactcccctgagccggactgctgccacgagc
tgctggggcacgtgcccatgctggccgaccgcaccttcgcgcagttctc
gcaggacattggcctggcgtccctgggggcctcggatgaggaaattgag
aagctgtccacgctgtactggttcacggtggagttcgggctgtgtaagc
agaacggggaggtgaaggcctatggtgccgggctgctgtcctcctacgg
ggagctcctgcactgcctgtctgaggagcctgagattcgggccttcgac
cctgaggctgcggccgtgcagccctaccaagaccagacgtaccagtcag
tctacttcgtgtctgagagcttcagtgacgccaaggacaagctcaggag
ctatgcctcacgcatccagcgccccttctccgtgaagttcgacccgtac
acgctggccatcgacgtgctggacagcccccaggccgtgcggcgctccc
tggagggtgtccaggatgagctggacacccttgcccatgcgctgagtgc
cattggctaaagcaggtttccccaactgacacaaaacgtgcaacttgaa
actccgcctggtctttccaggtctagaggggtaacactttgtactgcgt
ttggctccacgctcgatccactggcgagtgttagtaacagcactgttgc
ttcgtagcggagcatgacggccgtgggaactcctccttggtaacaagga
cccacggggccaaaagccacgcccacacgggcccgtcatgtgtgcaacc
ccagcacggcgactttactgcgaaacccactttaaagtgacattgaaac
tggtacccacacactggtgacaggctaaggatgcccttcaggtaccccg
aggtaacacgcgacactcgggatctgagaaggggactggggcttctata
aaagcgctcggtttaaaaagcttctatgcctgaataggtgaccggaggt
cggcacctttcctttgcaattactgaccacgccaccatggagaagccgc
ggggagtcaggtgcaccaatgggttctccgagcgggagctgccgcggcc
cggggccagcccgcctgccgagaagtcccggccgcccgaggccaagggc
gcacagccggccgacgcctggaaggcagggcggcaccgcagcgaggagg
aaaaccaggtgaacctccccaaactggcggctgcttactcgtccattct
gctctcgctgggcgaggacccccagcggcaggggctgctcaagacgccc
tggagggcggccaccgccatgcagtacttcaccaagggataccaggaga
ccatctcagatgtcctgaatgatgctatatttgatgaagatcatgacga
gatggtgattgtgaaggacatagatatgttctccatgtgtgagcatcac
cttgttccatttgtaggaagggtccatattggctatcttcctaacaagc
aagtccttggtctcagtaaacttgccaggattgtagaaatctacagtag
acgactacaagttcaagagcgcctcaccaaacagattgcggtggccatc
acagaagccttgcagcctgctggcgttggagtagtgattgaagcgacac
acatgtgcatggtaatgcgaggcgtgcagaaaatgaacagcaagactgt
cactagcaccatgctgggcgtgttccgggaagaccccaagactcgggag
gagttcctcacactaatcaggagctgaggccacctaatcaacctctgga
ttacaaaatttgtgaaagattgactggtattcttaactatgttgctcct
tttacgctatgtggatacgctgctttaatgcctttgtatcatgctattg
cttcccgtatggctttcattttctcctccttgtataaatcctggttgct
gtctctttatgaggagttgtggcccgttgtcaggcaacgtggcgtggtg
tgcactgtgtttgctgacgcaacccccactggttggggcattgccacca
cctgtcagctcctttccgggactttcgctttccccctccctattgccac
ggcggaactcatcgccgcctgccttgcccgctgctggacaggggctcgg
ctgttgggcactgacaattccgtggtgttgtcggggaaatcatcgtcct
ttcccatatgcagctcacagacatgataagatacattgatgagtttgga
caaaccacaactagaatgcagtgaaaaaaatgctttatttgtgaaattt
gtgatgctattgctttatttgtaaccattataagctgcaataaacaagt
taacaacaacaattgcattcattttatgtttcaggttcagggggaggtg
tgggaggttttttaaagcaagtaaaacctctacaaatgtggtattggcc
catctctatcggtatcgtagcataaccccttggggcctctaaacgggtc
ttgaggggttttttgtgcccctcgggccggattgctatctaccggcatt
ggcgcagaaaaaaatgcctgatgcgacgctgcgcgtcttatactcccac
atatgccagattcagcaacggatacggcttccccaacttgcccacttcc
atacgtgtcctccttaccagaaatttatccttaaggtcgtcagctatcc
tgcaggcgatctctcgatttcgatcaagacattcctttaatggtctttt
ctggacaccactaggggtcagaagtagttcatcaaactttcttccctcc
ctaatctcattggttaccttgggctatcgaaacttaattaaccagtcaa
gtcagctacttggcgagatcgacttgtctgggtttcgactacgctcaga
attgcgtcagtcaagttcgatctggtccttgctattgcacccgttctcc
gattacgagtttcatttaaatcatgtgagcaaaaggccagcaaaaggcc
aggaaccgtaaaaaggccgcgttgctggcgtttttccataggctccgcc
cccctgacgagcatcacaaaaatcgacgctcaagtcagaggtggcgaaa
cccgacaggactataaagataccaggcgtttccccctggaagctccctc
gtgcgctctcctgttccgaccctgccgcttaccggatacctgtccgcct
ttctcccttcgggaagcgtggcgctttctcatagctcacgctgtaggta
tctcagttcggtgtaggtcgttcgctccaagctgggctgtgtgcacgaa
ccccccgttcagcccgaccgctgcgccttatccggtaactatcgtcttg
agtccaacccggtaagacacgacttatcgccactggcagcagccactgg
taacaggattagcagagcgaggtatgtaggcggtgctacagagttcttg
aagtggtggcctaactacggctacactagaagaacagtatttggtatct
gcgctctgctgaagccagttaccttcggaaaaagagttggtagctcttg
atccggcaaacaaaccaccgctggtagcggtggtttttttgtttgcaag
cagcagattacgcgcagaaaaaaaggatctcaagaagatcctttgatct
tttctacggggtctgacgctcagtggaacgaaaactcacgttaagggat
tttggtcatgagattatcaaaaaggatcttcacctagatccttttaaat
taaaaatgaagttttaaatcaatctaaagtatatatgagtaaacttggt
ctgacagttaccaatgcttaatcagtgaggcacctatctcagcgatctg
tctatttcgttcatccatagttgcatttaaatttccgaactctccaagg
ccctcgtcggaaaatcttcaaacctttcgtccgatccatcttgcaggct
acctctcgaacgaactatcgcaagtctcttggccggccttgcgccttgg
ctattgcttggcagcgcctatcgccaggtattactccaatcccgaatat
ccgagatcgggatcacccgagagaagttcaacctacatcctcaatcccg
atctatccgagatccgaggaatatcgaaatcggggcgcgcctggtgtac
cgagaacgatcctctcagtgcgagtctcgacgatccatatcgttgcttg
gcagtcagccagtcggaatccagcttgggacccaggaagtccaatcgtc
agatattgtactcaagcctggtcacggcagcgtaccgatctgtttaaac
ctagatattgatagtctgatcggtcaacgtataatcgagtcctagcttt
tgcaaacatctatcaagagacaggatcagcaggaggctttcgcatgagt
attcaacatttccgtgtcgcccttattcccttttttgcggcattttgcc
ttcctgtttttgctcacccagaaacgctggtgaaagtaaaagatgctga
agatcagttgggtgcgcgagtgggttacatcgaactggatctcaacagc
ggtaagatccttgagagttttcgccccgaagaacgctttccaatgatga
gcacttttaaagttctgctatgtggcgcggtattatcccgtattgacgc
cgggcaagagcaactcggtcgccgcatacactattctcagaatgacttg
gttgagtattcaccagtcacagaaaagcatcttacggatggcatgacag
taagagaattatgcagtgctgccataaccatgagtgataacactgcggc
caacttacttctgacaacgattggaggaccgaaggagctaaccgctttt
ttgcacaacatgggggatcatgtaactcgccttgatcgttgggaaccgg
agctgaatgaagccataccaaacgacgagcgtgacaccacgatgcctgt
agcaatggcaacaaccttgcgtaaactattaactggcgaactacttact
ctagcttcccggcaacagttgatagactggatggaggcggataaagttg
caggaccacttctgcgctcggcccttccggctggctggtttattgctga
taaatctggagccggtgagcgtgggtctcgcggtatcattgcagcactg
gggccagatggtaagccctcccgtatcgtagttatctacacgacgggga
gtcaggcaactatggatgaacgaaatagacagatcgctgagataggtgc
ctcactgattaagcattggtaaccgattctaggtgcattggcgcagaaa
aaaatgcctgatgcgacgctgcgcgtcttatactcccacatatgccaga
ttcagcaacggatacggcttccccaacttgcccacttccatacgtgtcc
tccttaccagaaatttatccttaagatcccgaatcgtttaaactcgact
ctggctctatcgaatctccgtcgtttcgagcttacgcgaacagccgtgg
cgctcatttgctcgtcgggcatcgaatctcgtcagctatcgtcagctta
cctttttggca

Preferably, the genetic construct comprises a nucleic acid sequence substantially as set out in SEQ ID No: 18, or a fragment or variant thereof.

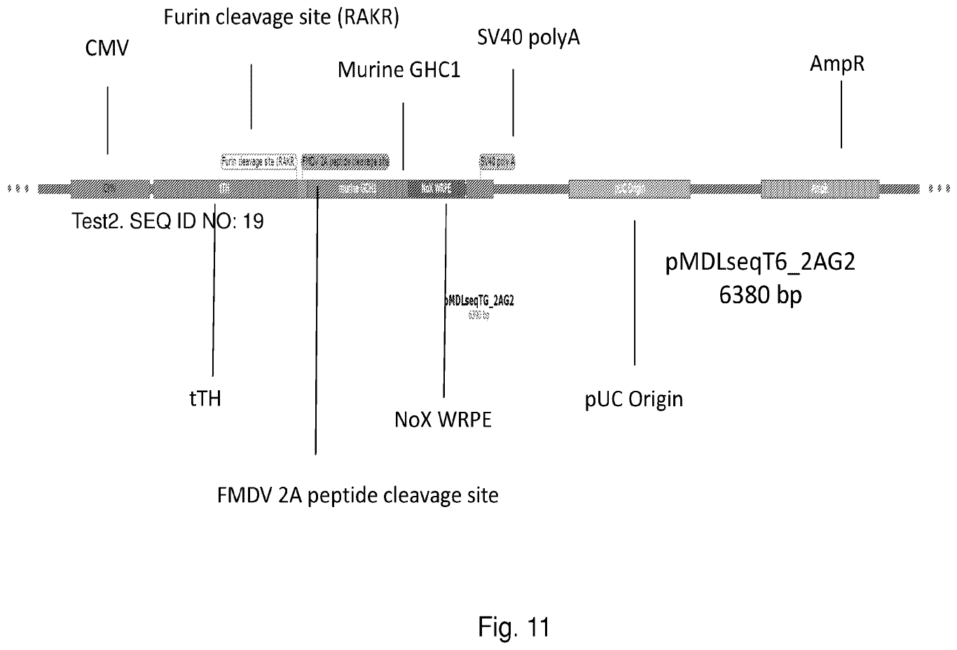

One embodiment of the genetic construct is shown in FIG. 11, and is referred to herein as SEQ ID No: 19. This particular embodiment includes a CMV promoter and murine GCH1; these features would be easily replaceable by the skilled person for other variants as disclosed herein. The murine form of GCH1 is to facilitate preclinical testing of the construct. The murine form of GCH1 could be easily replaced by a skilled person, for instance the murine form could be replaced by the human form of GCH1.

[SEQ ID NO: 19]
ggcgatcgcggctcccgacatcttggaccattagctccacaggtatctt
cttccctctagtggtcataacagcagcttcagctacctctcaattcaaa
aaacccctcaagacccgtttagaggccccaaggggttatgctatcaatc
gttgcgttacacacacaaaaaaccaacacacatccatcttcgatggata
gcgattttattatctaactgctgatcgagtgtagccagatctagtaatc
aattacggggtcattagttcatagcccatatatggagttccgcgttaca
taacttacggtaaatggcccgcctggctgaccgcccaacgacccccgcc
cattgacgtcaataatgacgtatgttcccatagtaacgccaatagggac
tttccattgacgtcaatgggtggagtatttacggtaaactgcccacttg
gcagtacatcaagtgtatcatatgccaagtacgccccctattgacgtca
atgacggtaaatggcccgcctggcattatgcccagtacatgaccttatg
ggactttcctacttggcagtacatctacgtattagtcatcgctattacc
atgctgatgcggttttggcagtacatcaatgggcgtggatagcggtttg
actcacggggatttccaagtctccaccccattgacgtcaatgggagttt
gttttggcaccaaaatcaacgggactttccaaaatgtcgtaacaactcc
gccccattgacgcaaatgggcggtaggcgtgtacggtgggaggtctata
taagcagagctggtttagtgaaccgtcagatcagatctagagatcccgg
gaccgccaccatgagccccgcggggcccaaggtcccctggttcccaaga
aaagtgtcagagctggacaagtgtcatcacctggtcaccaagttcgacc
ctgacctggacttggaccacccgggcttctcggaccaggtgtaccgcca
gcgcaggaagctgattgctgagatcgccttccagtacaggcacggcgac
ccgattccccgtgtggagtacaccgccgaggagattgccacctggaagg
aggtctacaccacgctgaagggcctctacgccacgcacgcctgcgggga
gcacctggaggcctttgctttgctggagcgcttcagcggctaccgggaa
gacaatatcccccagctggaggacgtctcccgcttcctgaaggagcgca
cgggcttccagctgcggcctgtggccggcctgctgtccgcccgggactt
cctggccagcctggccttccgcgtgttccagtgcacccagtatatccgc
cacgcgtcctcgcccatgcactcccctgagccggactgctgccacgagc
tgctggggcacgtgcccatgctggccgaccgcaccttcgcgcagttctc
gcaggacattggcctggcgtccctgggggcctcggatgaggaaattgag
aagctgtccacgctgtactggttcacggtggagttcgggctgtgtaagc
agaacggggaggtgaaggcctatggtgccgggctgctgtcctcctacgg
ggagctcctgcactgcctgtctgaggagcctgagattcgggccttcgac
cctgaggctgcggccgtgcagccctaccaagaccagacgtaccagtcag
tctacttcgtgtctgagagcttcagtgacgccaaggacaagctcaggag
ctatgcctcacgcatccagcgccccttctccgtgaagttcgacccgtac
acgctggccatcgacgtgctggacagcccccaggccgtgcggcgctccc
tggagggtgtccaggatgagctggacacccttgcccatgcgctgagtgc
cattggctaacgcgcgaaacgcgcgccggtgaaacagaccctgaacttt
gatctgctgaaactggcgggcgatgtggaaagcaacccgggcccgatgg
agaagccgcggggagtcaggtgcaccaatgggttctccgagcgggagct
gccgcggcccggggccagcccgcctgccgagaagtcccggccgcccgag
gccaagggcgcacagccggccgacgcctggaaggcagggcggcaccgca
gcgaggaggaaaaccaggtgaacctccccaaactggcggctgcttactc
gtccattctgctctcgctgggcgaggacccccagcggcaggggctgctc
aagacgccctggagggcggccaccgccatgcagtacttcaccaagggat
accaggagaccatctcagatgtcctgaatgatgctatatttgatgaaga
tcatgacgagatggtgattgtgaaggacatagatatgttctccatgtgt
gagcatcaccttgttccatttgtaggaagggtccatattggctatcttc
ctaacaagcaagtccttggtctcagtaaacttgccaggattgtagaaat
ctacagtagacgactacaagttcaagagcgcctcaccaaacagattgcg
gtggccatcacagaagccttgcagcctgctggcgttggagtagtgattg
aagcgacacacatgtgcatggtaatgcgaggcgtgcagaaaatgaacag
caagactgtcactagcaccatgctgggcgtgttccgggaagaccccaag
actcgggaggagttcctcacactaatcaggagctgaggccacctaatca
acctctggattacaaaatttgtgaaagattgactggtattcttaactat
gttgctccttttacgctatgtggatacgctgctttaatgcctttgtatc
atgctattgcttcccgtatggctttcattttctcctccttgtataaatc
ctggttgctgtctctttatgaggagttgtggcccgttgtcaggcaacgt
ggcgtggtgtgcactgtgtttgctgacgcaacccccactggttggggca
ttgccaccacctgtcagctcctttccgggactttcgctttccccctccc
tattgccacggcggaactcatcgccgcctgccttgcccgctgctggaca
ggggctcggctgttgggcactgacaattccgtggtgttgtcggggaaat
catcgtcctttcccatatgcagctcacagacatgataagatacattgat
gagtttggacaaaccacaactagaatgcagtgaaaaaaatgctttattt
gtgaaatttgtgatgctattgctttatttgtaaccattataagctgcaa
taaacaagttaacaacaacaattgcattcattttatgtttcaggttcag
ggggaggtgtgggaggttttttaaagcaagtaaaacctctacaaatgtg
gtattggcccatctctatcggtatcgtagcataaccccttggggcctct
aaacgggtcttgaggggttttttgtgcccctcgggccggattgctatct
accggcattggcgcagaaaaaaatgcctgatgcgacgctgcgcgtctta
tactcccacatatgccagattcagcaacggatacggcttccccaacttg
cccacttccatacgtgtcctccttaccagaaatttatccttaaggtcgt
cagctatcctgcaggcgatctctcgatttcgatcaagacattcctttaa
tggtcttttctggacaccactaggggtcagaagtagttcatcaaacttt
cttccctccctaatctcattggttaccttgggctatcgaaacttaatta
accagtcaagtcagctacttggcgagatcgacttgtctgggtttcgact
acgctcagaattgcgtcagtcaagttcgatctggtccttgctattgcac
ccgttctccgattacgagtttcatttaaatcatgtgagcaaaaggccag
caaaaggccaggaaccgtaaaaaggccgcgttgctggcgtttttccata
ggctccgcccccctgacgagcatcacaaaaatcgacgctcaagtcagag
gtggcgaaacccgacaggactataaagataccaggcgtttccccctgga
agctccctcgtgcgctctcctgttccgaccctgccgcttaccggatacc
tgtccgcctttctcccttcgggaagcgtggcgctttctcatagctcacg
ctgtaggtatctcagttcggtgtaggtcgttcgctccaagctgggctgt
gtgcacgaaccccccgttcagcccgaccgctgcgccttatccggtaact
atcgtcttgagtccaacccggtaagacacgacttatcgccactggcagc
agccactggtaacaggattagcagagcgaggtatgtaggcggtgctaca
gagttcttgaagtggtggcctaactacggctacactagaagaacagtat
ttggtatctgcgctctgctgaagccagttaccttcggaaaaagagttgg
tagctcttgatccggcaaacaaaccaccgctggtagcggtggttttttt
gtttgcaagcagcagattacgcgcagaaaaaaaggatctcaagaagatc
ctttgatcttttctacggggtctgacgctcagtggaacgaaaactcacg
ttaagggattttggtcatgagattatcaaaaaggatcttcacctagatc
cttttaaattaaaaatgaagttttaaatcaatctaaagtatatatgagt
aaacttggtctgacagttaccaatgcttaatcagtgaggcacctatctc
agcgatctgtctatttcgttcatccatagttgcatttaaatttccgaac
tctccaaggccctcgtcggaaaatcttcaaacctttcgtccgatccatc
ttgcaggctacctctcgaacgaactatcgcaagtctcttggccggcctt
gcgccttggctattgcttggcagcgcctatcgccaggtattactccaat
cccgaatatccgagatcgggatcacccgagagaagttcaacctacatcc
tcaatcccgatctatccgagatccgaggaatatcgaaatcggggcgcgc
ctggtgtaccgagaacgatcctctcagtgcgagtctcgacgatccatat
cgttgcttggcagtcagccagtcggaatccagcttgggacccaggaagt
ccaatcgtcagatattgtactcaagcctggtcacggcagcgtaccgatc
tgtttaaacctagatattgatagtctgatcggtcaacgtataatcgagt
cctagcttttgcaaacatctatcaagagacaggatcagcaggaggcttt
cgcatgagtattcaacatttccgtgtcgcccttattcccttttttgcgg
cattttgccttcctgtttttgctcacccagaaacgctggtgaaagtaaa
agatgctgaagatcagttgggtgcgcgagtgggttacatcgaactggat
ctcaacagcggtaagatccttgagagttttcgccccgaagaacgctttc
caatgatgagcacttttaaagttctgctatgtggcgcggtattatcccg
tattgacgccgggcaagagcaactcggtcgccgcatacactattctcag
aatgacttggttgagtattcaccagtcacagaaaagcatcttacggatg
gcatgacagtaagagaattatgcagtgctgccataaccatgagtgataa
cactgcggccaacttacttctgacaacgattggaggaccgaaggagcta
accgcttttttgcacaacatgggggatcatgtaactcgccttgatcgtt
gggaaccggagctgaatgaagccataccaaacgacgagcgtgacaccac
gatgcctgtagcaatggcaacaaccttgcgtaaactattaactggcgaa
ctacttactctagcttcccggcaacagttgatagactggatggaggcgg
ataaagttgcaggaccacttctgcgctcggcccttccggctggctggtt
tattgctgataaatctggagccggtgagcgtgggtctcgcggtatcatt
gcagcactggggccagatggtaagccctcccgtatcgtagttatctaca
cgacggggagtcaggcaactatggatgaacgaaatagacagatcgctga
gataggtgcctcactgattaagcattggtaaccgattctaggtgcattg
gcgcagaaaaaaatgcctgatgcgacgctgcgcgtcttatactcccaca
tatgccagattcagcaacggatacggcttccccaacttgcccacttcca
tacgtgtcctccttaccagaaatttatccttaagatcccgaatcgttta
aactcgactctggctctatcgaatctccgtcgtttcgagcttacgcgaa
cagccgtggcgctcatttgctcgtcgggcatcgaatctcgtcagctatc
gtcagcttacctttttggca

Preferably, the genetic construct comprises a nucleic acid sequence substantially as set out in SEQ ID No: 19, or a fragment or variant thereof.

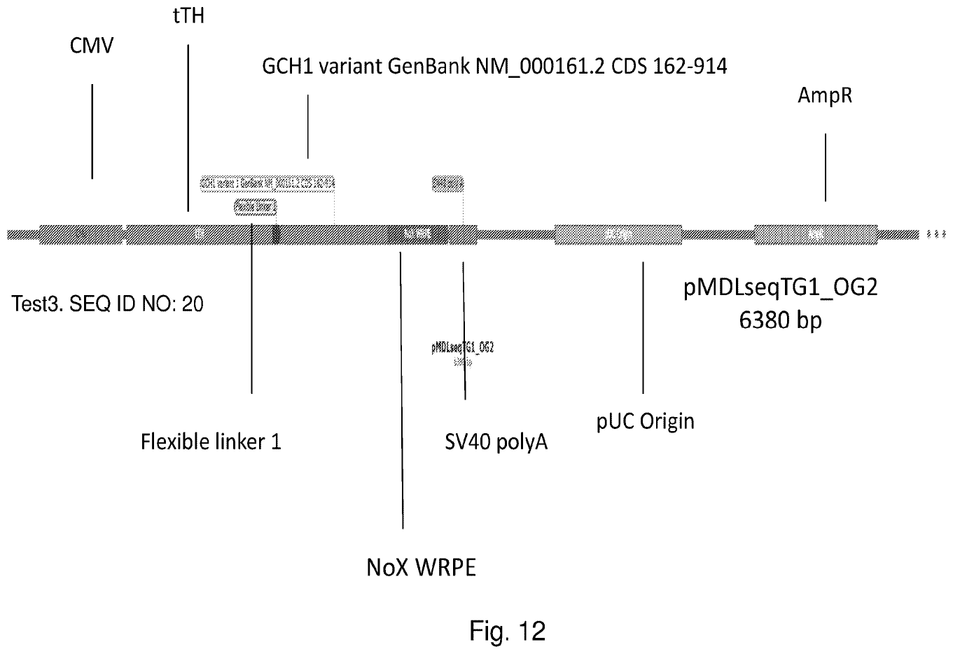

One embodiment of the genetic construct is shown in FIG. 12, and is referred to herein as SEQ ID No: 20. This particular embodiment includes a CMV promoter and murine GCH1; these features would be easily replaceable by the skilled person for other variants as disclosed herein. The murine form of GCH1 is to facilitate preclinical testing of the construct. The murine form of GCH1 could be easily replaced by a skilled person, for instance the murine form could be replaced by the human form of GCH1.

[SEQ ID NO: 20]
ggcgatcgcggctcccgacatcttggaccattagctccacaggtatctt
cttccctctagtggtcataacagcagcttcagctacctctcaattcaaa
aaacccctcaagacccgtttagaggccccaaggggttatgctatcaatc
gttgcgttacacacacaaaaaaccaacacacatccatcttcgatggata
gcgattttattatctaactgctgatcgagtgtagccagatctagtaatc
aattacggggtcattagttcatagcccatatatggagttccgcgttaca
taacttacggtaaatggcccgcctggctgaccgcccaacgacccccgcc
cattgacgtcaataatgacgtatgttcccatagtaacgccaatagggac
tttccattgacgtcaatgggtggagtatttacggtaaactgcccacttg
gcagtacatcaagtgtatcatatgccaagtacgccccctattgacgtca
atgacggtaaatggcccgcctggcattatgcccagtacatgaccttatg
ggactttcctacttggcagtacatctacgtattagtcatcgctattacc
atgctgatgcggttttggcagtacatcaatgggcgtggatagcggtttg
actcacggggatttccaagtctccaccccattgacgtcaatgggagttt
gttttggcaccaaaatcaacgggactttccaaaatgtcgtaacaactcc
gccccattgacgcaaatgggcggtaggcgtgtacggtgggaggtctata
taagcagagctggtttagtgaaccgtcagatcagatctagagatcccgg
gaccgccaccatgagccccgcggggcccaaggtcccctggttcccaaga
aaagtgtcagagctggacaagtgtcatcacctggtcaccaagttcgacc
ctgacctggacttggaccacccgggcttctcggaccaggtgtaccgcca
gcgcaggaagctgattgctgagatcgccttccagtacaggcacggcgac
ccgattccccgtgtggagtacaccgccgaggagattgccacctggaagg
aggtctacaccacgctgaagggcctctacgccacgcacgcctgcgggga
gcacctggaggcctttgctttgctggagcgcttcagcggctaccgggaa
gacaatatcccccagctggaggacgtctcccgcttcctgaaggagcgca
cgggcttccagctgcggcctgtggccggcctgctgtccgcccgggactt
cctggccagcctggccttccgcgtgttccagtgcacccagtatatccgc
cacgcgtcctcgcccatgcactcccctgagccggactgctgccacgagc
tgctggggcacgtgcccatgctggccgaccgcaccttcgcgcagttctc
gcaggacattggcctggcgtccctgggggcctcggatgaggaaattgag
aagctgtccacgctgtactggttcacggtggagttcgggctgtgtaagc
agaacggggaggtgaaggcctatggtgccgggctgctgtcctcctacgg
ggagctcctgcactgcctgtctgaggagcctgagattcgggccttcgac
cctgaggctgcggccgtgcagccctaccaagaccagacgtaccagtcag
tctacttcgtgtctgagagcttcagtgacgccaaggacaagctcaggag
ctatgcctcacgcatccagcgccccttctccgtgaagttcgacccgtac
acgctggccatcgacgtgctggacagcccccaggccgtgcggcgctccc
tggagggtgtccaggatgagctggacacccttgcccatgcgctgagtgc
cattggcggaggtggcgggtccgggggcgggggtagcggtggcgggggc
tccgccaccatggagaagggccctgtgcgggcaccggcggagaagccgc
ggggcgccaggtgcagcaatgggttccccgagcgggatccgccgcggcc
cgggcccagcaggccggcggagaagcccccgcggcccgaggccaagagc
gcgcagcccgcggacggctggaagggcgagcggccccgcagcgaggagg
ataacgagctgaacctccctaacctggcagccgcctactcgtccatcct
gagctcgctgggcgagaacccccagcggcaagggctgctcaagacgccc
tggagggcggcctcggccatgcagttcttcaccaagggctaccaggaga
ccatctcagatgtcctaaacgatgctatatttgatgaagatcatgatga
gatggtgattgtgaaggacatagacatgttttccatgtgtgagcatcac
ttggttccatttgttggaaaggtccatattggttatcttcctaacaagc
aagtccttggcctcagcaaacttgcgaggattgtagaaatctatagtag
aagactacaagttcaggagcgccttacaaaacaaattgctgtagcaatc
acggaagccttgcggcctgctggagtcggggtagtggttgaagcaacac
acatgtgtatggtaatgcgaggtgtacagaaaatgaacagcaaaactgt
gaccagcacaatgttgggtgtgttccgggaggatccaaagactcgggaa
gagttcctgactctcattaggagctgagccacctaatcaacctctggat
tacaaaatttgtgaaagattgactggtattcttaactatgttgctcctt
ttacgctatgtggatacgctgctttaatgcctttgtatcatgctattgc
ttcccgtatggctttcattttctcctccttgtataaatcctggttgctg
tctctttatgaggagttgtggcccgttgtcaggcaacgtggcgtggtgt
gcactgtgtttgctgacgcaacccccactggttggggcattgccaccac
ctgtcagctcctttccgggactttcgctttccccctccctattgccacg
gcggaactcatcgccgcctgccttgcccgctgctggacaggggctcggc
tgttgggcactgacaattccgtggtgttgtcggggaaatcatcgtcctt
tcccatatgcagctcacagacatgataagatacattgatgagtttggac
aaaccacaactagaatgcagtgaaaaaaatgctttatttgtgaaatttg
tgatgctattgctttatttgtaaccattataagctgcaataaacaagtt
aacaacaacaattgcattcattttatgtttcaggttcagggggaggtgt
gggaggttttttaaagcaagtaaaacctctacaaatgtggtattggccc
atctctatcggtatcgtagcataaccccttggggcctctaaacgggtct
tgaggggttttttgtgcccctcgggccggattgctatctaccggcattg
gcgcagaaaaaaatgcctgatgcgacgctgcgcgtcttatactcccaca
tatgccagattcagcaacggatacggcttccccaacttgcccacttcca
tacgtgtcctccttaccagaaatttatccttaaggtcgtcagctatcct
gcaggcgatctctcgatttcgatcaagacattcctttaatggtcttttc
tggacaccactaggggtcagaagtagttcatcaaactttcttccctccc
taatctcattggttaccttgggctatcgaaacttaattaaccagtcaag
tcagctacttggcgagatcgacttgtctgggtttcgactacgctcagaa
ttgcgtcagtcaagttcgatctggtccttgctattgcacccgttctccg
attacgagtttcatttaaatcatgtgagcaaaaggccagcaaaaggcca
ggaaccgtaaaaaggccgcgttgctggcgtttttccataggctccgccc
ccctgacgagcatcacaaaaatcgacgctcaagtcagaggtggcgaaac
ccgacaggactataaagataccaggcgtttccccctggaagctccctcg
tgcgctctcctgttccgaccctgccgcttaccggatacctgtccgcctt
tctcccttcgggaagcgtggcgctttctcatagctcacgctgtaggtat
ctcagttcggtgtaggtcgttcgctccaagctgggctgtgtgcacgaac
cccccgttcagcccgaccgctgcgccttatccggtaactatcgtcttga
gtccaacccggtaagacacgacttatcgccactggcagcagccactggt
aacaggattagcagagcgaggtatgtaggcggtgctacagagttcttga
agtggtggcctaactacggctacactagaagaacagtatttggtatctg
cgctctgctgaagccagttaccttcggaaaaagagttggtagctcttga
tccggcaaacaaaccaccgctggtagcggtggtttttttgtttgcaagc
agcagattacgcgcagaaaaaaaggatctcaagaagatcctttgatctt
ttctacggggtctgacgctcagtggaacgaaaactcacgttaagggatt
ttggtcatgagattatcaaaaaggatcttcacctagatccttttaaatt
aaaaatgaagttttaaatcaatctaaagtatatatgagtaaacttggtc
tgacagttaccaatgcttaatcagtgaggcacctatctcagcgatctgt
ctatttcgttcatccatagttgcatttaaatttccgaactctccaaggc
cctcgtcggaaaatcttcaaacctttcgtccgatccatcttgcaggcta
cctctcgaacgaactatcgcaagtctcttggccggccttgcgccttggc
tattgcttggcagcgcctatcgccaggtattactccaatcccgaatatc
cgagatcgggatcacccgagagaagttcaacctacatcctcaatcccga
tctatccgagatccgaggaatatcgaaatcggggcgcgcctggtgtacc
gagaacgatcctctcagtgcgagtctcgacgatccatatcgttgcttgg
cagtcagccagtcggaatccagcttgggacccaggaagtccaatcgtca
gatattgtactcaagcctggtcacggcagcgtaccgatctgtttaaacc
tagatattgatagtctgatcggtcaacgtataatcgagtcctagctttt
gcaaacatctatcaagagacaggatcagcaggaggctttcgcatgagta
ttcaacatttccgtgtcgcccttattcccttttttgcggcattttgcct
tcctgtttttgctcacccagaaacgctggtgaaagtaaaagatgctgaa
gatcagttgggtgcgcgagtgggttacatcgaactggatctcaacagcg
gtaagatccttgagagttttcgccccgaagaacgctttccaatgatgag
cacttttaaagttctgctatgtggcgcggtattatcccgtattgacgcc
gggcaagagcaactcggtcgccgcatacactattctcagaatgacttgg
ttgagtattcaccagtcacagaaaagcatcttacggatggcatgacagt
aagagaattatgcagtgctgccataaccatgagtgataacactgcggcc
aacttacttctgacaacgattggaggaccgaaggagctaaccgcttttt
tgcacaacatgggggatcatgtaactcgccttgatcgttgggaaccgga
gctgaatgaagccataccaaacgacgagcgtgacaccacgatgcctgta
gcaatggcaacaaccttgcgtaaactattaactggcgaactacttactc
tagcttcccggcaacagttgatagactggatggaggcggataaagttgc
aggaccacttctgcgctcggcccttccggctggctggtttattgctgat
aaatctggagccggtgagcgtgggtctcgcggtatcattgcagcactgg
ggccagatggtaagccctcccgtatcgtagttatctacacgacggggag
tcaggcaactatggatgaacgaaatagacagatcgctgagataggtgcc
tcactgattaagcattggtaaccgattctaggtgcattggcgcagaaaa
aaatgcctgatgcgacgctgcgcgtcttatactcccacatatgccagat
tcagcaacggatacggcttccccaacttgcccacttccatacgtgtcct
ccttaccagaaatttatccttaagatcccgaatcgtttaaactcgactc
tggctctatcgaatctccgtcgtttcgagcttacgcgaacagccgtggc
gctcatttgctcgtcgggcatcgaatctcgtcagctatcgtcagcttac
ctttttggca

Preferably, the genetic construct comprises a nucleic acid sequence substantially as set out in SEQ ID No: 20, or a fragment or variant thereof.

The inventors have created a series of recombinant expression vectors comprising the construct of the invention.

Thus, according to a second aspect, there is provided a recombinant vector comprising the genetic construct according to the first aspect.

The recombinant vector may be a recombinant AAV (rAAV) vector. The rAAV may be a naturally occurring vector or a vector with a hybrid AAV serotype. The rAAV may be AAV-1, AAV-2, AAV-3A, AAV-3B, AAV-4, AAV-5, AAV-6, AAV-7, AAV-8, AAV-9, AAV-10, and AAV-11. Preferably, the rAAV has tropism to neural tissue. In a preferred embodiment, the rAAV may be AAV1, AAV9, and more preferably AAV5.

The term “recombinant AAV (rAAV) vector” as used herein can mean a recombinant AAV-derived nucleic acid containing at least one terminal repeat sequence.

In an embodiment, the vector may be an AAV1 vector, comprising a human synapsin 1 promoter, a sequence encoding human truncated TH, an IRES, a sequence encoding human GCH1, a sequence encoding WPRE, a sequence encoding a poly A tail. The following sequence, referred to herein as SEQ ID NO: 13, depicts such a vector. This particular embodiment includes a CMV promoter, a CMV enchancer, an EMCV IRES, and a SV40 poly A tail. The individual features would be easily replaceable by the skilled person for alternatives as disclosed herein.

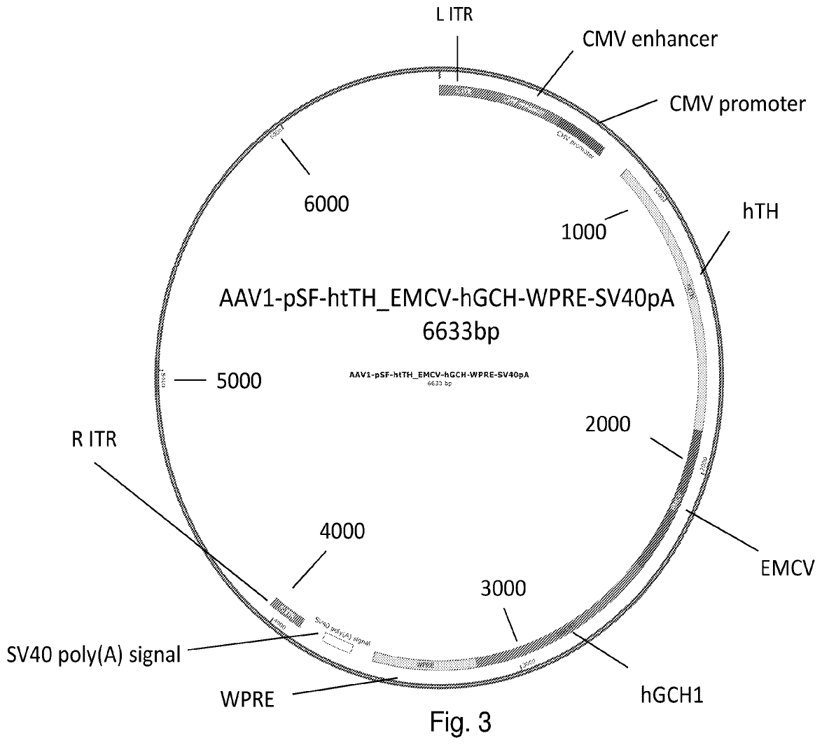

A map showing the features of a plasmid comprising SEQ ID NO: 13 is shown in FIG. 3.

[SEQ ID NO: 13]
CCTGCAGGCAGCTGCGCGCTCGCTCGCTCACTGAGGCCGCCCGGGCGTC
GGGCGACCTTTGGTCGCCCGGCCTCAGTGAGCGAGCGAGCGCGCAGAGA
GGGAGTGGCCAACTCCATCACTAGGGGTTCCTGCGGCCGGTCGCGTACT
AGTAATCAATTACGGGGTCATTAGTTCATAGCCCATATATGGAGTTCCG
CGTTACATAACTTACGGTAAATGGCCCGCCTGGCTGACCGCCCAACGAC
CCCCGCCCATTGACGTCAATAATGACGTATGTTCCCATAGTAACGCCAA
TAGGGACTTTCCATTGACGTCAATGGGTGGAGTATTTACGGTAAACTGC
CCACTTGGCAGTACATCAAGTGTATCATATGCCAAGTACGCCCCCTATT
GACGTCAATGACGGTAAATGGCCCGCCTGGCATTATGCCCAGTACATGA
CCTTATGGGACTTTCCTACTTGGCAGTACATCTACGTATTAGTCATCGC
TATTACCATGCTGATGCGGTTTTGGCAGTACATCAATGGGCGTGGATAG
CGGTTTGACTCACGGGGATTTCCAAGTCTCCACCCCATTGACGTCAATG
GGAGTTTGTTTTGGCACCAAAATCAACGGGACTTTCCAAAATGTCGTAA
CAACTCCGCCCCATTGACGCAAATGGGCGGTAGGCGTGTACGGTGGGAG
GTCTATATAAGCAGAGCTGGTTTAGTGAACCGTCAGATCAGATCTTTGT
CGATCCTACCATCCACTCGACACACCCGCCAGCGGCCGCTGCCAAGCTT
CCGAGCTCTCGAATTCAAAGGAGGTACCCACCATGGCCACCATGAGCCC
CGCGGGGCCCAAGGTCCCCTGGTTCCCAAGAAAAGTGTCAGAGCTGGAC
AAGTGTCATCACCTGGTCACCAAGTTCGACCCTGACCTGGACTTGGACC
ACCCGGGCTTCTCGGACCAGGTGTACCGCCAGCGCAGGAAGCTGATTGC
TGAGATCGCCTTCCAGTACAGGCACGGCGACCCGATTCCCCGTGTGGAG
TACACCGCCGAGGAGATTGCCACCTGGAAGGAGGTCTACACCACGCTGA
AGGGCCTCTACGCCACGCACGCCTGCGGGGAGCACCTGGAGGCCTTTGC
TTTGCTGGAGCGCTTCAGCGGCTACCGGGAAGACAATATCCCCCAGCTG
GAGGACGTCTCCCGCTTCCTGAAGGAGCGCACGGGCTTCCAGCTGCGGC
CTGTGGCCGGCCTGCTGTCCGCCCGGGACTTCCTGGCCAGCCTGGCCTT
CCGCGTGTTCCAGTGCACCCAGTATATCCGCCACGCGTCCTCGCCCATG
CACTCCCCTGAGCCGGACTGCTGCCACGAGCTGCTGGGGCACGTGCCCA
TGCTGGCCGACCGCACCTTCGCGCAGTTCTCGCAGGACATTGGCCTGGC
GTCCCTGGGGGCCTCGGATGAGGAAATTGAGAAGCTGTCCACGCTGTAC
TGGTTCACGGTGGAGTTCGGGCTGTGTAAGCAGAACGGGGAGGTGAAGG
CCTATGGTGCCGGGCTGCTGTCCTCCTACGGGGAGCTCCTGCACTGCCT
GTCTGAGGAGCCTGAGATTCGGGCCTTCGACCCTGAGGCTGCGGCCGTG
CAGCCCTACCAAGACCAGACGTACCAGTCAGTCTACTTCGTGTCTGAGA
GCTTCAGTGACGCCAAGGACAAGCTCAGGAGCTATGCCTCACGCATCCA
GCGCCCCTTCTCCGTGAAGTTCGACCCGTACACGCTGGCCATCGACGTG
CTGGACAGCCCCCAGGCCGTGCGGCGCTCCCTGGAGGGTGTCCAGGATG
AGCTGGACACCCTTGCCCATGCGCTGAGTGCCATTGGCTAAACGTTACT
GGCCGAAGCCGCTTGGAATAAGGCCGGTGTGCGTTTGTCTATATGTTAT
TTTCCACCATATTGCCGTCTTTTGGCAATGTGAGGGCCCGGAAACCTGG
CCCTGTCTTCTTGACGAGCATTCCTAGGGGTCTTTCCCCTCTCGCCAAA
GGAATGCAAGGTCTGTTGAATGTCGTGAAGGAAGCAGTTCCTCTGGAAG
CTTCTTGAAGACAAACAACGTCTGTAGCGACCCTTTGCAGGCAGCGGAA
CCCCCCACCTGGCGACAGGTGCCTCTGCGGCCAAAAGCCACGTGTATAA
GATACACCTGCAAAGGCGGCACAACCCCAGTGCCACGTTGTGAGTTGGA
TAGTTGTGGAAAGAGTCAAATGGCTCCCCTCAAGCGTATTCAACAAGGG
GCTGAAGGATGCCCAGAAGGTACCCCATTGTATGGGATCTGATCTGGGG
CCTCGGTGCACATGCTTTTCATGTGTTTAGTCGAGGTTAAAAAACGTCT
AGGCCCCCCGAACCACGGGGACGTGGTTTTCCTTTGAAAAACACGATGA
TAATAGCCACCATGGAGAAGGGCCCTGTGCGGGCACCGGCGGAGAAGCC
GCGGGGCGCCAGGTGCAGCAATGGGTTCCCCGAGCGGGATCCGCCGCGG
CCCGGGCCCAGCAGGCCGGCGGAGAAGCCCCCGCGGCCCGAGGCCAAGA
GCGCGCAGCCCGCGGACGGCTGGAAGGGCGAGCGGCCCCGCAGCGAGGA
GGATAACGAGCTGAACCTCCCTAACCTGGCAGCCGCCTACTCGTCCATC
CTGAGCTCGCTGGGCGAGAACCCCCAGCGGCAAGGGCTGCTCAAGACGC
CCTGGAGGGCGGCCTCGGCCATGCAGTTCTTCACCAAGGGCTACCAGGA
GACCATCTCAGATGTCCTAAACGATGCTATATTTGATGAAGATCATGAT
GAGATGGTGATTGTGAAGGACATAGACATGTTTTCCATGTGTGAGCATC
ACTTGGTTCCATTTGTTGGAAAGGTCCATATTGGTTATCTTCCTAACAA
GCAAGTCCTTGGCCTCAGCAAACTTGCGAGGATTGTAGAAATCTATAGT
AGAAGACTACAAGTTCAGGAGCGCCTTACAAAACAAATTGCTGTAGCAA
TCACGGAAGCCTTGCGGCCTGCTGGAGTCGGGGTAGTGGTTGAAGCAAC
ACACATGTGTATGGTAATGCGAGGTGTACAGAAAATGAACAGCAAAACT
GTGACCAGCACAATGTTGGGTGTGTTCCGGGAGGATCCAAAGACTCGGG
AAGAGTTCCTGACTCTCATTAGGAGCTGAGCCACCTAATCAACCTCTGG
ATTACAAAATTTGTGAAAGATTGACTGGTATTCTTAACTATGTTGCTCC
TTTTACGCTATGTGGATACGCTGCTTTAATGCCTTTGTATCATGCTATT
GCTTCCCGTATGGCTTTCATTTTCTCCTCCTTGTATAAATCCTGGTTGC
TGTCTCTTTATGAGGAGTTGTGGCCCGTTGTCAGGCAACGTGGCGTGGT
GTGCACTGTGTTTGCTGACGCAACCCCCACTGGTTGGGGCATTGCCACC
ACCTGTCAGCTCCTTTCCGGGACTTTCGCTTTCCCCCTCCCTATTGCCA
CGGCGGAACTCATCGCCGCCTGCCTTGCCCGCTGCTGGACAGGGGCTCG
GCTGTTGGGCACTGACAATTCCGTGGTGTTGTCGGGGAAATCATCGTCC
TTTCCCTGGCTGACTGATACAATCGATTTCTGGATCCGCAGGCCTCTGC
TAGCTTGACTGACTGAGATACAGCGTACCTTCAGCTCACAGACATGATA
AGATACATTGATGAGTTTGGACAAACCACAACTAGAATGCAGTGAAAAA
AATGCTTTATTTGTGAAATTTGTGATGCTATTGCTTTATTTGTAACCAT
TATAAGCTGCAATAAACAAGTTAACAACAACAATTGCATTCATTTTATG
TTTCAGGTTCAGGGGGAGGTGTGGGAGGTTTTTTAAGCTTAACGCGGTA
ACCACGTGCGGACCCAACGGCCGCAGGAACCCCTAGTGATGGAGTTGGC
CACTCCCTCTCTGCGCGCTCGCTCGCTCACTGAGGCCGGGCGACCAAAG
GTCGCCCGACGCCCGGGCTTTGCCCGGGCGGCCTCAGTGAGCGAGCGAG
CGCGCAGCTGCCTGCAGGGGCGCCTGATGCGGTATTTTCTCCTTACGCA
TCTGTGCGGTATTTCACACCGCATACGTCAAAGCAACCATAGTACGCGC
CCTGTAGCGGCGCATTAAGCGCGGCGGGTGTGGTGGTTACGCGCAGCGT
GACCGCTACACTTGCCAGCGCCCTAGCGCCCGCTCCTTTCGCTTTCTTC
CCTTCCTTTCTCGCCACGTTCGCCGGCTTTCCCCGTCAAGCTCTAAATC
GGGGGCTCCCTTTAGGGTTCCGATTTAGTGCTTTACGGCACCTCGACCC
CAAAAAACTTGATTTGGGTGATGGTTCACGTAGTGGGCCATCGCCCTGA
TAGACGGTTTTTCGCCCTTTGACGTTGGAGTCCACGTTCTTTAATAGTG
GACTCTTGTTCCAAACTGGAACAACACTCAACCCTATCTCGGGCTATTC
TTTTGATTTATAAGGGATTTTGCCGATTTCGGCCTATTGGTTAAAAAAT
GAGCTGATTTAACAAAAATTTAACGCGAATTTTAACAAAATATTAACGT
TTACAATTTTATGGTGCACTCTCAGTACAATCTGCTCTGATGCCGCATA
GTTAAGCCAGCCCCGACACCCGCCAACACCCGCTGACGCGCCCTGACGG
GCTTGTCTGCTCCCGGCATCCGCTTACAGACAAGCTGTGACCGTCTCCG
GGAGCTGCATGTGTCAGAGGTTTTCACCGTCATCACCGAAACGCGCGAG
ACGAAAGGGCCTCGTGATACGCCTATTTTTATAGGTTAATGTCATGATA
ATAATGGTTTCTTAGACGTCAGGTGGCACTTTTCGGGGAAATGTGCGCG
GAACCCCTATTTGTTTATTTTTCTAAATACATTCAAATATGTATCCGCT
CATGAGACAATAACCCTGATAAATGCTTCAATAATATTGAAAAAGGAAG
AGTATGAGTATTCAACATTTCCGTGTCGCCCTTATTCCCTTTTTTGCGG
CATTTTGCCTTCCTGTTTTTGCTCACCCAGAAACGCTGGTGAAAGTAAA
AGATGCTGAAGATCAGTTGGGTGCACGAGTGGGTTACATCGAACTGGAT
CTCAACAGCGGTAAGATCCTTGAGAGTTTTCGCCCCGAAGAACGTTTTC
CAATGATGAGCACTTTTAAAGTTCTGCTATGTGGCGCGGTATTATCCCG
TATTGACGCCGGGCAAGAGCAACTCGGTCGCCGCATACACTATTCTCAG
AATGACTTGGTTGAGTACTCACCAGTCACAGAAAAGCATCTTACGGATG
GCATGACAGTAAGAGAATTATGCAGTGCTGCCATAACCATGAGTGATAA
CACTGCGGCCAACTTACTTCTGACAACGATCGGAGGACCGAAGGAGCTA
ACCGCTTTTTTGCACAACATGGGGGATCATGTAACTCGCCTTGATCGTT
GGGAACCGGAGCTGAATGAAGCCATACCAAACGACGAGCGTGACACCAC
GATGCCTGTAGCAATGGCAACAACGTTGCGCAAACTATTAACTGGCGAA
CTACTTACTCTAGCTTCCCGGCAACAATTAATAGACTGGATGGAGGCGG
ATAAAGTTGCAGGACCACTTCTGCGCTCGGCCCTTCCGGCTGGCTGGTT
TATTGCTGATAAATCTGGAGCCGGTGAGCGTGGGTCTCGCGGTATCATT
GCAGCACTGGGGCCAGATGGTAAGCCCTCCCGTATCGTAGTTATCTACA
CGACGGGGAGTCAGGCAACTATGGATGAACGAAATAGACAGATCGCTGA
GATAGGTGCCTCACTGATTAAGCATTGGTAACTGTCAGACCAAGTTTAC
TCATATATACTTTAGATTGATTTAAAACTTCATTTTTAATTTAAAAGGA
TCTAGGTGAAGATCCTTTTTGATAATCTCATGACCAAAATCCCTTAACG
TGAGTTTTCGTTCCACTGAGCGTCAGACCCCGTAGAAAAGATCAAAGGA
TCTTCTTGAGATCCTTTTTTTCTGCGCGTAATCTGCTGCTTGCAAACAA
AAAAACCACCGCTACCAGCGGTGGTTTGTTTGCCGGATCAAGAGCTACC
AACTCTTTTTCCGAAGGTAACTGGCTTCAGCAGAGCGCAGATACCAAAT
ACTGTCCTTCTAGTGTAGCCGTAGTTAGGCCACCACTTCAAGAACTCTG
TAGCACCGCCTACATACCTCGCTCTGCTAATCCTGTTACCAGTGGCTGC
TGCCAGTGGCGATAAGTCGTGTCTTACCGGGTTGGACTCAAGACGATAG
TTACCGGATAAGGCGCAGCGGTCGGGCTGAACGGGGGGTTCGTGCACAC
AGCCCAGCTTGGAGCGAACGACCTACACCGAACTGAGATACCTACAGCG
TGAGCTATGAGAAAGCGCCACGCTTCCCGAAGGGAGAAAGGCGGACAGG
TATCCGGTAAGCGGCAGGGTCGGAACAGGAGAGCGCACGAGGGAGCTTC
CAGGGGGAAACGCCTGGTATCTTTATAGTCCTGTCGGGTTTCGCCACCT
CTGACTTGAGCGTCGATTTTTGTGATGCTCGTCAGGGGGGCGGAGCCTA
TGGAAAAACGCCAGCAACGCGGCCTTTTTACGGTTCCTGGCCTTTTGCT
GGCCTTTTGCTCACATGT

Preferably, the vector comprises a nucleic acid sequence substantially as set out in SEQ ID No: 13, or a fragment or variant thereof.

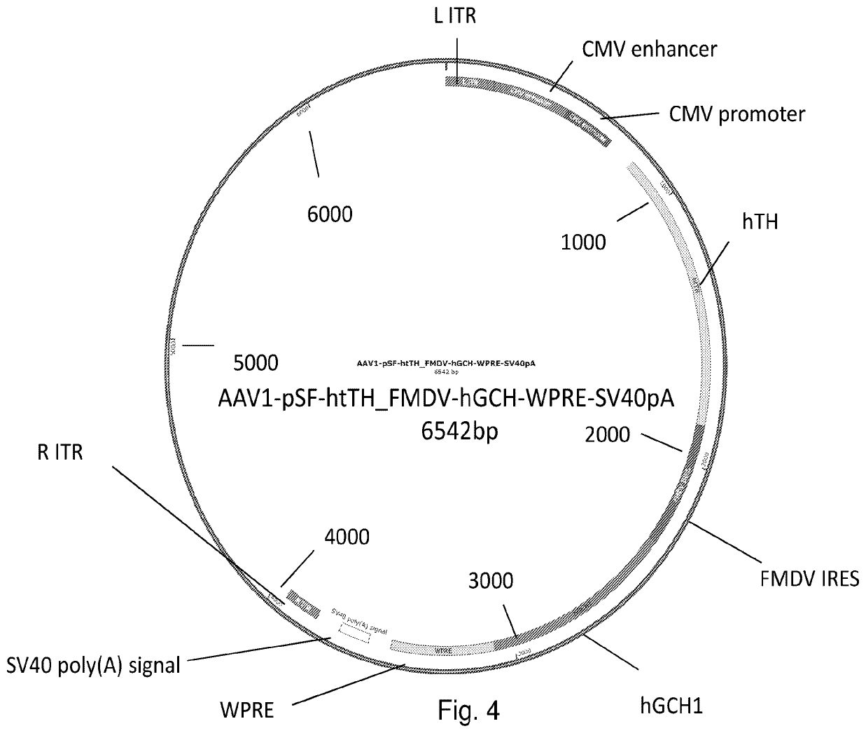

The following sequence, referred to herein as SEQ ID NO: 14, depicts a vector similar to SEQ ID NO: 13, but this particular embodiment includes an FMDV IRES instead of the EMCV IRES. A map showing the features of a plasmid comprising SEQ ID NO: 14 is shown in FIG. 4.

[SEQ ID NO: 14]
CCTGCAGGCAGCTGCGCGCTCGCTCGCTCACTGAGGCCGCCCGGGCGTC
GGGCGACCTTTGGTCGCCCGGCCTCAGTGAGCGAGCGAGCGCGCAGAGA
GGGAGTGGCCAACTCCATCACTAGGGGTTCCTGCGGCCGGTCGCGTACT
AGTAATCAATTACGGGGTCATTAGTTCATAGCCCATATATGGAGTTCCG
CGTTACATAACTTACGGTAAATGGCCCGCCTGGCTGACCGCCCAACGAC
CCCCGCCCATTGACGTCAATAATGACGTATGTTCCCATAGTAACGCCAA
TAGGGACTTTCCATTGACGTCAATGGGTGGAGTATTTACGGTAAACTGC
CCACTTGGCAGTACATCAAGTGTATCATATGCCAAGTACGCCCCCTATT
GACGTCAATGACGGTAAATGGCCCGCCTGGCATTATGCCCAGTACATGA
CCTTATGGGACTTTCCTACTTGGCAGTACATCTACGTATTAGTCATCGC
TATTACCATGCTGATGCGGTTTTGGCAGTACATCAATGGGCGTGGATAG
CGGTTTGACTCACGGGGATTTCCAAGTCTCCACCCCATTGACGTCAATG
GGAGTTTGTTTTGGCACCAAAATCAACGGGACTTTCCAAAATGTCGTAA
CAACTCCGCCCCATTGACGCAAATGGGCGGTAGGCGTGTACGGTGGGAG
GTCTATATAAGCAGAGCTGGTTTAGTGAACCGTCAGATCAGATCTTTGT
CGATCCTACCATCCACTCGACACACCCGCCAGCGGCCGCTGCCAAGCTT
CCGAGCTCTCGAATTCAAAGGAGGTACCCACCATGGCCACCATGAGCCC
CGCGGGGCCCAAGGTCCCCTGGTTCCCAAGAAAAGTGTCAGAGCTGGAC
AAGTGTCATCACCTGGTCACCAAGTTCGACCCTGACCTGGACTTGGACC
ACCCGGGCTTCTCGGACCAGGTGTACCGCCAGCGCAGGAAGCTGATTGC
TGAGATCGCCTTCCAGTACAGGCACGGCGACCCGATTCCCCGTGTGGAG
TACACCGCCGAGGAGATTGCCACCTGGAAGGAGGTCTACACCACGCTGA
AGGGCCTCTACGCCACGCACGCCTGCGGGGAGCACCTGGAGGCCTTTGC
TTTGCTGGAGCGCTTCAGCGGCTACCGGGAAGACAATATCCCCCAGCTG
GAGGACGTCTCCCGCTTCCTGAAGGAGCGCACGGGCTTCCAGCTGCGGC
CTGTGGCCGGCCTGCTGTCCGCCCGGGACTTCCTGGCCAGCCTGGCCTT
CCGCGTGTTCCAGTGCACCCAGTATATCCGCCACGCGTCCTCGCCCATG
CACTCCCCTGAGCCGGACTGCTGCCACGAGCTGCTGGGGCACGTGCCCA
TGCTGGCCGACCGCACCTTCGCGCAGTTCTCGCAGGACATTGGCCTGGC
GTCCCTGGGGGCCTCGGATGAGGAAATTGAGAAGCTGTCCACGCTGTAC
TGGTTCACGGTGGAGTTCGGGCTGTGTAAGCAGAACGGGGAGGTGAAGG
CCTATGGTGCCGGGCTGCTGTCCTCCTACGGGGAGCTCCTGCACTGCCT
GTCTGAGGAGCCTGAGATTCGGGCCTTCGACCCTGAGGCTGCGGCCGTG
CAGCCCTACCAAGACCAGACGTACCAGTCAGTCTACTTCGTGTCTGAGA
GCTTCAGTGACGCCAAGGACAAGCTCAGGAGCTATGCCTCACGCATCCA
GCGCCCCTTCTCCGTGAAGTTCGACCCGTACACGCTGGCCATCGACGTG
CTGGACAGCCCCCAGGCCGTGCGGCGCTCCCTGGAGGGTGTCCAGGATG
AGCTGGACACCCTTGCCCATGCGCTGAGTGCCATTGGCTAAAGCAGGTT
TCCCCAACTGACACAAAACGTGCAACTTGAAACTCCGCCTGGTCTTTCC
AGGTCTAGAGGGGTAACACTTTGTACTGCGTTTGGCTCCACGCTCGATC
CACTGGCGAGTGTTAGTAACAGCACTGTTGCTTCGTAGCGGAGCATGAC
GGCCGTGGGAACTCCTCCTTGGTAACAAGGACCCACGGGGCCAAAAGCC
ACGCCCACACGGGCCCGTCATGTGTGCAACCCCAGCACGGCGACTTTAC
TGCGAAACCCACTTTAAAGTGACATTGAAACTGGTACCCACACACTGGT
GACAGGCTAAGGATGCCCTTCAGGTACCCCGAGGTAACACGCGACACTC
GGGATCTGAGAAGGGGACTGGGGCTTCTATAAAAGCGCTCGGTTTAAAA
AGCTTCTATGCCTGAATAGGTGACCGGAGGTCGGCACCTTTCCTTTGCA
ATTACTGACCACGCCACCATGGAGAAGGGCCCTGTGCGGGCACCGGCGG
AGAAGCCGCGGGGCGCCAGGTGCAGCAATGGGTTCCCCGAGCGGGATCC
GCCGCGGCCCGGGCCCAGCAGGCCGGCGGAGAAGCCCCCGCGGCCCGAG
GCCAAGAGCGCGCAGCCCGCGGACGGCTGGAAGGGCGAGCGGCCCCGCA
GCGAGGAGGATAACGAGCTGAACCTCCCTAACCTGGCAGCCGCCTACTC
GTCCATCCTGAGCTCGCTGGGCGAGAACCCCCAGCGGCAAGGGCTGCTC
AAGACGCCCTGGAGGGCGGCCTCGGCCATGCAGTTCTTCACCAAGGGCT
ACCAGGAGACCATCTCAGATGTCCTAAACGATGCTATATTTGATGAAGA
TCATGATGAGATGGTGATTGTGAAGGACATAGACATGTTTTCCATGTGT
GAGCATCACTTGGTTCCATTTGTTGGAAAGGTCCATATTGGTTATCTTC
CTAACAAGCAAGTCCTTGGCCTCAGCAAACTTGCGAGGATTGTAGAAAT
CTATAGTAGAAGACTACAAGTTCAGGAGCGCCTTACAAAACAAATTGCT
GTAGCAATCACGGAAGCCTTGCGGCCTGCTGGAGTCGGGGTAGTGGTTG
AAGCAACACACATGTGTATGGTAATGCGAGGTGTACAGAAAATGAACAG
CAAAACTGTGACCAGCACAATGTTGGGTGTGTTCCGGGAGGATCCAAAG
ACTCGGGAAGAGTTCCTGACTCTCATTAGGAGCTGAGCCACCTAATCAA
CCTCTGGATTACAAAATTTGTGAAAGATTGACTGGTATTCTTAACTATG
TTGCTCCTTTTACGCTATGTGGATACGCTGCTTTAATGCCTTTGTATCA
TGCTATTGCTTCCCGTATGGCTTTCATTTTCTCCTCCTTGTATAAATCC
TGGTTGCTGTCTCTTTATGAGGAGTTGTGGCCCGTTGTCAGGCAACGTG
GCGTGGTGTGCACTGTGTTTGCTGACGCAACCCCCACTGGTTGGGGCAT
TGCCACCACCTGTCAGCTCCTTTCCGGGACTTTCGCTTTCCCCCTCCCT
ATTGCCACGGCGGAACTCATCGCCGCCTGCCTTGCCCGCTGCTGGACAG
GGGCTCGGCTGTTGGGCACTGACAATTCCGTGGTGTTGTCGGGGAAATC
ATCGTCCTTTCCCTGGCTGACTGATACAATCGATTTCTGGATCCGCAGG
CCTCTGCTAGCTTGACTGACTGAGATACAGCGTACCTTCAGCTCACAGA
CATGATAAGATACATTGATGAGTTTGGACAAACCACAACTAGAATGCAG
TGAAAAAAATGCTTTATTTGTGAAATTTGTGATGCTATTGCTTTATTTG
TAACCATTATAAGCTGCAATAAACAAGTTAACAACAACAATTGCATTCA
TTTTATGTTTCAGGTTCAGGGGGAGGTGTGGGAGGTTTTTTAAGCTTAA
CGCGGTAACCACGTGCGGACCCAACGGCCGCAGGAACCCCTAGTGATGG
AGTTGGCCACTCCCTCTCTGCGCGCTCGCTCGCTCACTGAGGCCGGGCG
ACCAAAGGTCGCCCGACGCCCGGGCTTTGCCCGGGCGGCCTCAGTGAGC
GAGCGAGCGCGCAGCTGCCTGCAGGGGCGCCTGATGCGGTATTTTCTCC
TTACGCATCTGTGCGGTATTTCACACCGCATACGTCAAAGCAACCATAG
TACGCGCCCTGTAGCGGCGCATTAAGCGCGGCGGGTGTGGTGGTTACGC
GCAGCGTGACCGCTACACTTGCCAGCGCCCTAGCGCCCGCTCCTTTCGC
TTTCTTCCCTTCCTTTCTCGCCACGTTCGCCGGCTTTCCCCGTCAAGCT
CTAAATCGGGGGCTCCCTTTAGGGTTCCGATTTAGTGCTTTACGGCACC
TCGACCCCAAAAAACTTGATTTGGGTGATGGTTCACGTAGTGGGCCATC
GCCCTGATAGACGGTTTTTCGCCCTTTGACGTTGGAGTCCACGTTCTTT
AATAGTGGACTCTTGTTCCAAACTGGAACAACACTCAACCCTATCTCGG
GCTATTCTTTTGATTTATAAGGGATTTTGCCGATTTCGGCCTATTGGTT
AAAAAATGAGCTGATTTAACAAAAATTTAACGCGAATTTTAACAAAATA
TTAACGTTTACAATTTTATGGTGCACTCTCAGTACAATCTGCTCTGATG
CCGCATAGTTAAGCCAGCCCCGACACCCGCCAACACCCGCTGACGCGCC
CTGACGGGCTTGTCTGCTCCCGGCATCCGCTTACAGACAAGCTGTGACC
GTCTCCGGGAGCTGCATGTGTCAGAGGTTTTCACCGTCATCACCGAAAC
GCGCGAGACGAAAGGGCCTCGTGATACGCCTATTTTTATAGGTTAATGT
CATGATAATAATGGTTTCTTAGACGTCAGGTGGCACTTTTCGGGGAAAT
GTGCGCGGAACCCCTATTTGTTTATTTTTCTAAATACATTCAAATATGT
ATCCGCTCATGAGACAATAACCCTGATAAATGCTTCAATAATATTGAAA
AAGGAAGAGTATGAGTATTCAACATTTCCGTGTCGCCCTTATTCCCTTT
TTTGCGGCATTTTGCCTTCCTGTTTTTGCTCACCCAGAAACGCTGGTGA
AAGTAAAAGATGCTGAAGATCAGTTGGGTGCACGAGTGGGTTACATCGA
ACTGGATCTCAACAGCGGTAAGATCCTTGAGAGTTTTCGCCCCGAAGAA
CGTTTTCCAATGATGAGCACTTTTAAAGTTCTGCTATGTGGCGCGGTAT
TATCCCGTATTGACGCCGGGCAAGAGCAACTCGGTCGCCGCATACACTA
TTCTCAGAATGACTTGGTTGAGTACTCACCAGTCACAGAAAAGCATCTT
ACGGATGGCATGACAGTAAGAGAATTATGCAGTGCTGCCATAACCATGA
GTGATAACACTGCGGCCAACTTACTTCTGACAACGATCGGAGGACCGAA
GGAGCTAACCGCTTTTTTGCACAACATGGGGGATCATGTAACTCGCCTT
GATCGTTGGGAACCGGAGCTGAATGAAGCCATACCAAACGACGAGCGTG
ACACCACGATGCCTGTAGCAATGGCAACAACGTTGCGCAAACTATTAAC
TGGCGAACTACTTACTCTAGCTTCCCGGCAACAATTAATAGACTGGATG
GAGGCGGATAAAGTTGCAGGACCACTTCTGCGCTCGGCCCTTCCGGCTG
GCTGGTTTATTGCTGATAAATCTGGAGCCGGTGAGCGTGGGTCTCGCGG
TATCATTGCAGCACTGGGGCCAGATGGTAAGCCCTCCCGTATCGTAGTT
ATCTACACGACGGGGAGTCAGGCAACTATGGATGAACGAAATAGACAGA
TCGCTGAGATAGGTGCCTCACTGATTAAGCATTGGTAACTGTCAGACCA
AGTTTACTCATATATACTTTAGATTGATTTAAAACTTCATTTTTAATTT
AAAAGGATCTAGGTGAAGATCCTTTTTGATAATCTCATGACCAAAATCC
CTTAACGTGAGTTTTCGTTCCACTGAGCGTCAGACCCCGTAGAAAAGAT
CAAAGGATCTTCTTGAGATCCTTTTTTTCTGCGCGTAATCTGCTGCTTG
CAAACAAAAAAACCACCGCTACCAGCGGTGGTTTGTTTGCCGGATCAAG
AGCTACCAACTCTTTTTCCGAAGGTAACTGGCTTCAGCAGAGCGCAGAT
ACCAAATACTGTCCTTCTAGTGTAGCCGTAGTTAGGCCACCACTTCAAG
AACTCTGTAGCACCGCCTACATACCTCGCTCTGCTAATCCTGTTACCAG
TGGCTGCTGCCAGTGGCGATAAGTCGTGTCTTACCGGGTTGGACTCAAG
ACGATAGTTACCGGATAAGGCGCAGCGGTCGGGCTGAACGGGGGGTTCG
TGCACACAGCCCAGCTTGGAGCGAACGACCTACACCGAACTGAGATACC
TACAGCGTGAGCTATGAGAAAGCGCCACGCTTCCCGAAGGGAGAAAGGC
GGACAGGTATCCGGTAAGCGGCAGGGTCGGAACAGGAGAGCGCACGAGG
GAGCTTCCAGGGGGAAACGCCTGGTATCTTTATAGTCCTGTCGGGTTTC
GCCACCTCTGACTTGAGCGTCGATTTTTGTGATGCTCGTCAGGGGGGCG
GAGCCTATGGAAAAACGCCAGCAACGCGGCCTTTTTACGGTTCCTGGCC
TTTTGCTGGCCTTTTGCTCACATGT

Preferably, the vector comprises a nucleic acid sequence substantially as set out in SEQ ID No: 14, or a fragment or variant thereof.

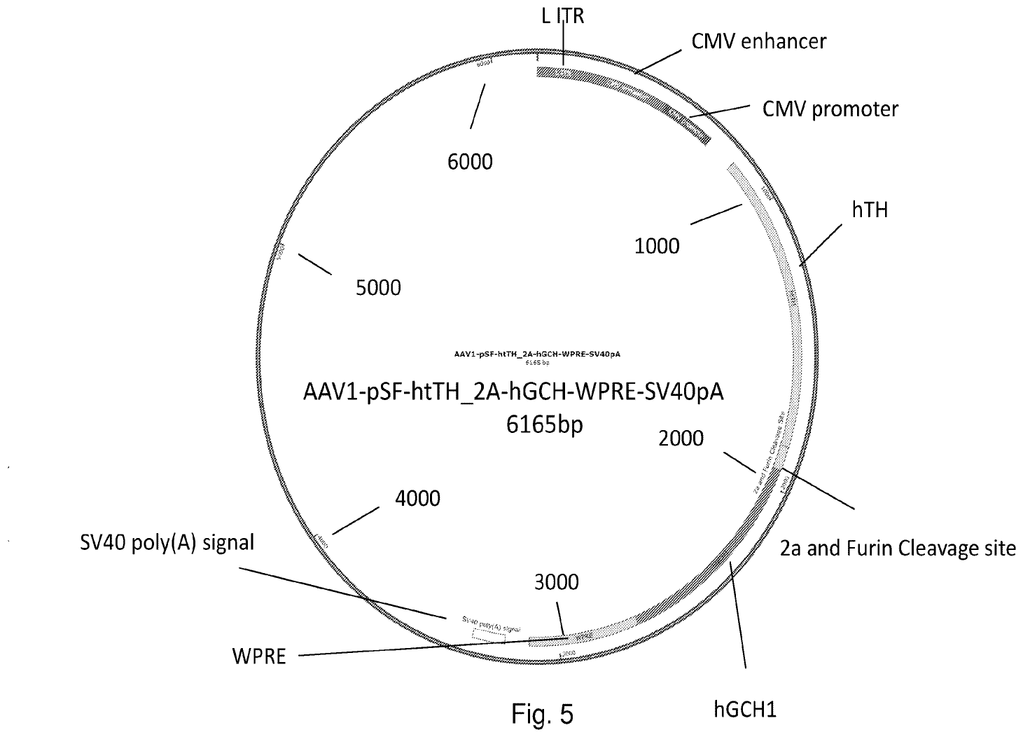

The following sequence, referred to herein as SEQ ID NO: 15, depicts a vector similar to SEQ ID NO: 13, but this particular embodiment includes a Furin cleavage site and a viral 2A peptide spacer, instead of the EMCV IRES. A map showing the features of a plasmid comprising SEQ ID NO: 15 is shown in FIG. 5.

[SEQ ID NO: 15]
CCTGCAGGCAGCTGCGCGCTCGCTCGCTCACTGAGGCCGCCCGGGCGTC
GGGCGACCTTTGGTCGCCCGGCCTCAGTGAGCGAGCGAGCGCGCAGAGA
GGGAGTGGCCAACTCCATCACTAGGGGTTCCTGCGGCCGGTCGCGTACT
AGTAATCAATTACGGGGTCATTAGTTCATAGCCCATATATGGAGTTCCG
CGTTACATAACTTACGGTAAATGGCCCGCCTGGCTGACCGCCCAACGAC
CCCCGCCCATTGACGTCAATAATGACGTATGTTCCCATAGTAACGCCAA
TAGGGACTTTCCATTGACGTCAATGGGTGGAGTATTTACGGTAAACTGC
CCACTTGGCAGTACATCAAGTGTATCATATGCCAAGTACGCCCCCTATT
GACGTCAATGACGGTAAATGGCCCGCCTGGCATTATGCCCAGTACATGA
CCTTATGGGACTTTCCTACTTGGCAGTACATCTACGTATTAGTCATCGC
TATTACCATGCTGATGCGGTTTTGGCAGTACATCAATGGGCGTGGATAG
CGGTTTGACTCACGGGGATTTCCAAGTCTCCACCCCATTGACGTCAATG
GGAGTTTGTTTTGGCACCAAAATCAACGGGACTTTCCAAAATGTCGTAA
CAACTCCGCCCCATTGACGCAAATGGGCGGTAGGCGTGTACGGTGGGAG
GTCTATATAAGCAGAGCTGGTTTAGTGAACCGTCAGATCAGATCTTTGT
CGATCCTACCATCCACTCGACACACCCGCCAGCGGCCGCTGCCAAGCTT
CCGAGCTCTCGAATTCAAAGGAGGTACCCACCATGGCCACCATGAGCCC
CGCGGGGCCCAAGGTCCCCTGGTTCCCAAGAAAAGTGTCAGAGCTGGAC
AAGTGTCATCACCTGGTCACCAAGTTCGACCCTGACCTGGACTTGGACC
ACCCGGGCTTCTCGGACCAGGTGTACCGCCAGCGCAGGAAGCTGATTGC
TGAGATCGCCTTCCAGTACAGGCACGGCGACCCGATTCCCCGTGTGGAG
TACACCGCCGAGGAGATTGCCACCTGGAAGGAGGTCTACACCACGCTGA
AGGGCCTCTACGCCACGCACGCCTGCGGGGAGCACCTGGAGGCCTTTGC
TTTGCTGGAGCGCTTCAGCGGCTACCGGGAAGACAATATCCCCCAGCTG
GAGGACGTCTCCCGCTTCCTGAAGGAGCGCACGGGCTTCCAGCTGCGGC
CTGTGGCCGGCCTGCTGTCCGCCCGGGACTTCCTGGCCAGCCTGGCCTT
CCGCGTGTTCCAGTGCACCCAGTATATCCGCCACGCGTCCTCGCCCATG
CACTCCCCTGAGCCGGACTGCTGCCACGAGCTGCTGGGGCACGTGCCCA
TGCTGGCCGACCGCACCTTCGCGCAGTTCTCGCAGGACATTGGCCTGGC
GTCCCTGGGGGCCTCGGATGAGGAAATTGAGAAGCTGTCCACGCTGTAC
TGGTTCACGGTGGAGTTCGGGCTGTGTAAGCAGAACGGGGAGGTGAAGG
CCTATGGTGCCGGGCTGCTGTCCTCCTACGGGGAGCTCCTGCACTGCCT
GTCTGAGGAGCCTGAGATTCGGGCCTTCGACCCTGAGGCTGCGGCCGTG
CAGCCCTACCAAGACCAGACGTACCAGTCAGTCTACTTCGTGTCTGAGA
GCTTCAGTGACGCCAAGGACAAGCTCAGGAGCTATGCCTCACGCATCCA
GCGCCCCTTCTCCGTGAAGTTCGACCCGTACACGCTGGCCATCGACGTG
CTGGACAGCCCCCAGGCCGTGCGGCGCTCCCTGGAGGGTGTCCAGGATG
AGCTGGACACCCTTGCCCATGCGCTGAGTGCCATTGGCTAACGCGCGAA
ACGCGCGCCGGTGAAACAGACCCTGAACTTTGATCTGCTGAAACTGGCG
GGCGATGTGGAAAGCAACCCGGGCCCGGCCACCATGGAGAAGGGCCCTG
TGCGGGCACCGGCGGAGAAGCCGCGGGGCGCCAGGTGCAGCAATGGGTT
CCCCGAGCGGGATCCGCCGCGGCCCGGGCCCAGCAGGCCGGCGGAGAAG
CCCCCGCGGCCCGAGGCCAAGAGCGCGCAGCCCGCGGACGGCTGGAAGG
GCGAGCGGCCCCGCAGCGAGGAGGATAACGAGCTGAACCTCCCTAACCT
GGCAGCCGCCTACTCGTCCATCCTGAGCTCGCTGGGCGAGAACCCCCAG
CGGCAAGGGCTGCTCAAGACGCCCTGGAGGGCGGCCTCGGCCATGCAGT
TCTTCACCAAGGGCTACCAGGAGACCATCTCAGATGTCCTAAACGATGC
TATATTTGATGAAGATCATGATGAGATGGTGATTGTGAAGGACATAGAC
ATGTTTTCCATGTGTGAGCATCACTTGGTTCCATTTGTTGGAAAGGTCC
ATATTGGTTATCTTCCTAACAAGCAAGTCCTTGGCCTCAGCAAACTTGC
GAGGATTGTAGAAATCTATAGTAGAAGACTACAAGTTCAGGAGCGCCTT
ACAAAACAAATTGCTGTAGCAATCACGGAAGCCTTGCGGCCTGCTGGAG
TCGGGGTAGTGGTTGAAGCAACACACATGTGTATGGTAATGCGAGGTGT
ACAGAAAATGAACAGCAAAACTGTGACCAGCACAATGTTGGGTGTGTTC
CGGGAGGATCCAAAGACTCGGGAAGAGTTCCTGACTCTCATTAGGAGCT
GAGCCACCTAATCAACCTCTGGATTACAAAATTTGTGAAAGATTGACTG
GTATTCTTAACTATGTTGCTCCTTTTACGCTATGTGGATACGCTGCTTT
AATGCCTTTGTATCATGCTATTGCTTCCCGTATGGCTTTCATTTTCTCC
TCCTTGTATAAATCCTGGTTGCTGTCTCTTTATGAGGAGTTGTGGCCCG
TTGTCAGGCAACGTGGCGTGGTGTGCACTGTGTTTGCTGACGCAACCCC
CACTGGTTGGGGCATTGCCACCACCTGTCAGCTCCTTTCCGGGACTTTC
GCTTTCCCCCTCCCTATTGCCACGGCGGAACTCATCGCCGCCTGCCTTG
CCCGCTGCTGGACAGGGGCTCGGCTGTTGGGCACTGACAATTCCGTGGT
GTTGTCGGGGAAATCATCGTCCTTTCCCTGGCTGACTGATACAATCGAT
TTCTGGATCCGCAGGCCTCTGCTAGCTTGACTGACTGAGATACAGCGTA
CCTTCAGCTCACAGACATGATAAGATACATTGATGAGTTTGGACAAACC
ACAACTAGAATGCAGTGAAAAAAATGCTTTATTTGTGAAATTTGTGATG
CTATTGCTTTATTTGTAACCATTATAAGCTGCAATAAACAAGTTAACAA
CAACAATTGCATTCATTTTATGTTTCAGGTTCAGGGGGAGGTGTGGGAG
GTTTTTTAAGCTTAACGCGGTAACCACGTGCGGACCCAACGGCCGCAGG
AACCCCTAGTGATGGAGTTGGCCACTCCCTCTCTGCGCGCTCGCTCGCT
CACTGAGGCCGGGCGACCAAAGGTCGCCCGACGCCCGGGCTTTGCCCGG
GCGGCCTCAGTGAGCGAGCGAGCGCGCAGCTGCCTGCAGGGGCGCCTGA
TGCGGTATTTTCTCCTTACGCATCTGTGCGGTATTTCACACCGCATACG
TCAAAGCAACCATAGTACGCGCCCTGTAGCGGCGCATTAAGCGCGGCGG
GTGTGGTGGTTACGCGCAGCGTGACCGCTACACTTGCCAGCGCCCTAGC
GCCCGCTCCTTTCGCTTTCTTCCCTTCCTTTCTCGCCACGTTCGCCGGC
TTTCCCCGTCAAGCTCTAAATCGGGGGCTCCCTTTAGGGTTCCGATTTA
GTGCTTTACGGCACCTCGACCCCAAAAAACTTGATTTGGGTGATGGTTC
ACGTAGTGGGCCATCGCCCTGATAGACGGTTTTTCGCCCTTTGACGTTG
GAGTCCACGTTCTTTAATAGTGGACTCTTGTTCCAAACTGGAACAACAC
TCAACCCTATCTCGGGCTATTCTTTTGATTTATAAGGGATTTTGCCGAT
TTCGGCCTATTGGTTAAAAAATGAGCTGATTTAACAAAAATTTAACGCG
AATTTTAACAAAATATTAACGTTTACAATTTTATGGTGCACTCTCAGTA
CAATCTGCTCTGATGCCGCATAGTTAAGCCAGCCCCGACACCCGCCAAC
ACCCGCTGACGCGCCCTGACGGGCTTGTCTGCTCCCGGCATCCGCTTAC
AGACAAGCTGTGACCGTCTCCGGGAGCTGCATGTGTCAGAGGTTTTCAC
CGTCATCACCGAAACGCGCGAGACGAAAGGGCCTCGTGATACGCCTATT
TTTATAGGTTAATGTCATGATAATAATGGTTTCTTAGACGTCAGGTGGC
ACTTTTCGGGGAAATGTGCGCGGAACCCCTATTTGTTTATTTTTCTAAA
TACATTCAAATATGTATCCGCTCATGAGACAATAACCCTGATAAATGCT
TCAATAATATTGAAAAAGGAAGAGTATGAGTATTCAACATTTCCGTGTC
GCCCTTATTCCCTTTTTTGCGGCATTTTGCCTTCCTGTTTTTGCTCACC
CAGAAACGCTGGTGAAAGTAAAAGATGCTGAAGATCAGTTGGGTGCACG
AGTGGGTTACATCGAACTGGATCTCAACAGCGGTAAGATCCTTGAGAGT
TTTCGCCCCGAAGAACGTTTTCCAATGATGAGCACTTTTAAAGTTCTGC
TATGTGGCGCGGTATTATCCCGTATTGACGCCGGGCAAGAGCAACTCGG
TCGCCGCATACACTATTCTCAGAATGACTTGGTTGAGTACTCACCAGTC
ACAGAAAAGCATCTTACGGATGGCATGACAGTAAGAGAATTATGCAGTG
CTGCCATAACCATGAGTGATAACACTGCGGCCAACTTACTTCTGACAAC
GATCGGAGGACCGAAGGAGCTAACCGCTTTTTTGCACAACATGGGGGAT
CATGTAACTCGCCTTGATCGTTGGGAACCGGAGCTGAATGAAGCCATAC
CAAACGACGAGCGTGACACCACGATGCCTGTAGCAATGGCAACAACGTT
GCGCAAACTATTAACTGGCGAACTACTTACTCTAGCTTCCCGGCAACAA
TTAATAGACTGGATGGAGGCGGATAAAGTTGCAGGACCACTTCTGCGCT
CGGCCCTTCCGGCTGGCTGGTTTATTGCTGATAAATCTGGAGCCGGTGA
GCGTGGGTCTCGCGGTATCATTGCAGCACTGGGGCCAGATGGTAAGCCC
TCCCGTATCGTAGTTATCTACACGACGGGGAGTCAGGCAACTATGGATG
AACGAAATAGACAGATCGCTGAGATAGGTGCCTCACTGATTAAGCATTG
GTAACTGTCAGACCAAGTTTACTCATATATACTTTAGATTGATTTAAAA
CTTCATTTTTAATTTAAAAGGATCTAGGTGAAGATCCTTTTTGATAATC
TCATGACCAAAATCCCTTAACGTGAGTTTTCGTTCCACTGAGCGTCAGA
CCCCGTAGAAAAGATCAAAGGATCTTCTTGAGATCCTTTTTTTCTGCGC
GTAATCTGCTGCTTGCAAACAAAAAAACCACCGCTACCAGCGGTGGTTT
GTTTGCCGGATCAAGAGCTACCAACTCTTTTTCCGAAGGTAACTGGCTT
CAGCAGAGCGCAGATACCAAATACTGTCCTTCTAGTGTAGCCGTAGTTA
GGCCACCACTTCAAGAACTCTGTAGCACCGCCTACATACCTCGCTCTGC
TAATCCTGTTACCAGTGGCTGCTGCCAGTGGCGATAAGTCGTGTCTTAC
CGGGTTGGACTCAAGACGATAGTTACCGGATAAGGCGCAGCGGTCGGGC
TGAACGGGGGGTTCGTGCACACAGCCCAGCTTGGAGCGAACGACCTACA
CCGAACTGAGATACCTACAGCGTGAGCTATGAGAAAGCGCCACGCTTCC
CGAAGGGAGAAAGGCGGACAGGTATCCGGTAAGCGGCAGGGTCGGAACA
GGAGAGCGCACGAGGGAGCTTCCAGGGGGAAACGCCTGGTATCTTTATA
GTCCTGTCGGGTTTCGCCACCTCTGACTTGAGCGTCGATTTTTGTGATG
CTCGTCAGGGGGGCGGAGCCTATGGAAAAACGCCAGCAACGCGGCCTTT
TTACGGTTCCTGGCCTTTTGCTGGCCTTTTGCTCACATGT

Preferably, the vector comprises a nucleic acid sequence substantially as set out in SEQ ID No: 15, or a fragment or variant thereof.

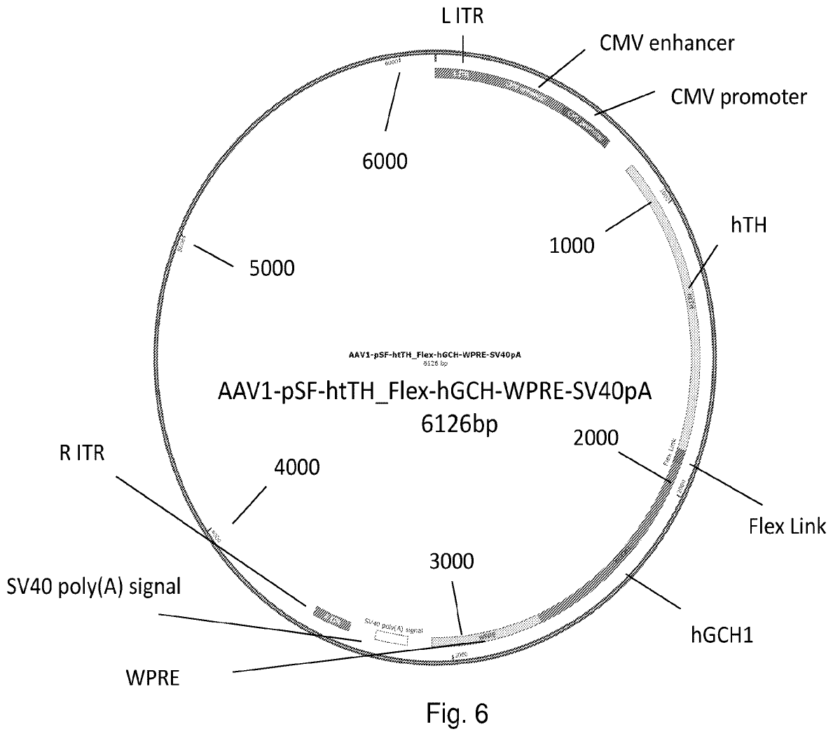

The following sequence, referred to herein as SEQ ID NO:16, depicts a vector similar to SEQ ID NO: 13, but this particular embodiment includes a flexible linker, instead of the EMCV IRES. A map showing the features of a plasmid comprising SEQ ID NO: 16 is shown in FIG. 6.

[SEQ ID NO: 16]
CCTGCAGGCAGCTGCGCGCTCGCTCGCTCACTGAGGCCGCCCGGGCGTC
GGGCGACCTTTGGTCGCCCGGCCTCAGTGAGCGAGCGAGCGCGCAGAGA
GGGAGTGGCCAACTCCATCACTAGGGGTTCCTGCGGCCGGTCGCGTACT
AGTAATCAATTACGGGGTCATTAGTTCATAGCCCATATATGGAGTTCCG
CGTTACATAACTTACGGTAAATGGCCCGCCTGGCTGACCGCCCAACGAC
CCCCGCCCATTGACGTCAATAATGACGTATGTTCCCATAGTAACGCCAA
TAGGGACTTTCCATTGACGTCAATGGGTGGAGTATTTACGGTAAACTGC
CCACTTGGCAGTACATCAAGTGTATCATATGCCAAGTACGCCCCCTATT
GACGTCAATGACGGTAAATGGCCCGCCTGGCATTATGCCCAGTACATGA
CCTTATGGGACTTTCCTACTTGGCAGTACATCTACGTATTAGTCATCGC
TATTACCATGCTGATGCGGTTTTGGCAGTACATCAATGGGCGTGGATAG
CGGTTTGACTCACGGGGATTTCCAAGTCTCCACCCCATTGACGTCAATG
GGAGTTTGTTTTGGCACCAAAATCAACGGGACTTTCCAAAATGTCGTAA
CAACTCCGCCCCATTGACGCAAATGGGCGGTAGGCGTGTACGGTGGGAG
GTCTATATAAGCAGAGCTGGTTTAGTGAACCGTCAGATCAGATCTTTGT
CGATCCTACCATCCACTCGACACACCCGCCAGCGGCCGCTGCCAAGCTT
CCGAGCTCTCGAATTCAAAGGAGGTACCCACCATGGCCACCATGAGCCC
CGCGGGGCCCAAGGTCCCCTGGTTCCCAAGAAAAGTGTCAGAGCTGGAC
AAGTGTCATCACCTGGTCACCAAGTTCGACCCTGACCTGGACTTGGACC
ACCCGGGCTTCTCGGACCAGGTGTACCGCCAGCGCAGGAAGCTGATTGC
TGAGATCGCCTTCCAGTACAGGCACGGCGACCCGATTCCCCGTGTGGAG
TACACCGCCGAGGAGATTGCCACCTGGAAGGAGGTCTACACCACGCTGA
AGGGCCTCTACGCCACGCACGCCTGCGGGGAGCACCTGGAGGCCTTTGC
TTTGCTGGAGCGCTTCAGCGGCTACCGGGAAGACAATATCCCCCAGCTG
GAGGACGTCTCCCGCTTCCTGAAGGAGCGCACGGGCTTCCAGCTGCGGC
CTGTGGCCGGCCTGCTGTCCGCCCGGGACTTCCTGGCCAGCCTGGCCTT
CCGCGTGTTCCAGTGCACCCAGTATATCCGCCACGCGTCCTCGCCCATG
CACTCCCCTGAGCCGGACTGCTGCCACGAGCTGCTGGGGCACGTGCCCA
TGCTGGCCGACCGCACCTTCGCGCAGTTCTCGCAGGACATTGGCCTGGC
GTCCCTGGGGGCCTCGGATGAGGAAATTGAGAAGCTGTCCACGCTGTAC
TGGTTCACGGTGGAGTTCGGGCTGTGTAAGCAGAACGGGGAGGTGAAGG
CCTATGGTGCCGGGCTGCTGTCCTCCTACGGGGAGCTCCTGCACTGCCT
GTCTGAGGAGCCTGAGATTCGGGCCTTCGACCCTGAGGCTGCGGCCGTG
CAGCCCTACCAAGACCAGACGTACCAGTCAGTCTACTTCGTGTCTGAGA
GCTTCAGTGACGCCAAGGACAAGCTCAGGAGCTATGCCTCACGCATCCA
GCGCCCCTTCTCCGTGAAGTTCGACCCGTACACGCTGGCCATCGACGTG
CTGGACAGCCCCCAGGCCGTGCGGCGCTCCCTGGAGGGTGTCCAGGATG
AGCTGGACACCCTTGCCCATGCGCTGAGTGCCATTGGCTAAGGAGGTGG
CGGGTCCGGGGGCGGGGGTAGCGGTGGCGGGGGCTCCGCCACCATGGAG
AAGGGCCCTGTGCGGGCACCGGCGGAGAAGCCGCGGGGCGCCAGGTGCA
GCAATGGGTTCCCCGAGCGGGATCCGCCGCGGCCCGGGCCCAGCAGGCC
GGCGGAGAAGCCCCCGCGGCCCGAGGCCAAGAGCGCGCAGCCCGCGGAC
GGCTGGAAGGGCGAGCGGCCCCGCAGCGAGGAGGATAACGAGCTGAACC
TCCCTAACCTGGCAGCCGCCTACTCGTCCATCCTGAGCTCGCTGGGCGA
GAACCCCCAGCGGCAAGGGCTGCTCAAGACGCCCTGGAGGGCGGCCTCG
GCCATGCAGTTCTTCACCAAGGGCTACCAGGAGACCATCTCAGATGTCC
TAAACGATGCTATATTTGATGAAGATCATGATGAGATGGTGATTGTGAA
GGACATAGACATGTTTTCCATGTGTGAGCATCACTTGGTTCCATTTGTT
GGAAAGGTCCATATTGGTTATCTTCCTAACAAGCAAGTCCTTGGCCTCA
GCAAACTTGCGAGGATTGTAGAAATCTATAGTAGAAGACTACAAGTTCA
GGAGCGCCTTACAAAACAAATTGCTGTAGCAATCACGGAAGCCTTGCGG
CCTGCTGGAGTCGGGGTAGTGGTTGAAGCAACACACATGTGTATGGTAA
TGCGAGGTGTACAGAAAATGAACAGCAAAACTGTGACCAGCACAATGTT
GGGTGTGTTCCGGGAGGATCCAAAGACTCGGGAAGAGTTCCTGACTCTC
ATTAGGAGCTGAGCCACCTAATCAACCTCTGGATTACAAAATTTGTGAA
AGATTGACTGGTATTCTTAACTATGTTGCTCCTTTTACGCTATGTGGAT
ACGCTGCTTTAATGCCTTTGTATCATGCTATTGCTTCCCGTATGGCTTT
CATTTTCTCCTCCTTGTATAAATCCTGGTTGCTGTCTCTTTATGAGGAG
TTGTGGCCCGTTGTCAGGCAACGTGGCGTGGTGTGCACTGTGTTTGCTG
ACGCAACCCCCACTGGTTGGGGCATTGCCACCACCTGTCAGCTCCTTTC
CGGGACTTTCGCTTTCCCCCTCCCTATTGCCACGGCGGAACTCATCGCC
GCCTGCCTTGCCCGCTGCTGGACAGGGGCTCGGCTGTTGGGCACTGACA
ATTCCGTGGTGTTGTCGGGGAAATCATCGTCCTTTCCCTGGCTGACTGA
TACAATCGATTTCTGGATCCGCAGGCCTCTGCTAGCTTGACTGACTGAG
ATACAGCGTACCTTCAGCTCACAGACATGATAAGATACATTGATGAGTT
TGGACAAACCACAACTAGAATGCAGTGAAAAAAATGCTTTATTTGTGAA
ATTTGTGATGCTATTGCTTTATTTGTAACCATTATAAGCTGCAATAAAC
AAGTTAACAACAACAATTGCATTCATTTTATGTTTCAGGTTCAGGGGGA
GGTGTGGGAGGTTTTTTAAGCTTAACGCGGTAACCACGTGCGGACCCAA
CGGCCGCAGGAACCCCTAGTGATGGAGTTGGCCACTCCCTCTCTGCGCG
CTCGCTCGCTCACTGAGGCCGGGCGACCAAAGGTCGCCCGACGCCCGGG
CTTTGCCCGGGCGGCCTCAGTGAGCGAGCGAGCGCGCAGCTGCCTGCAG
GGGCGCCTGATGCGGTATTTTCTCCTTACGCATCTGTGCGGTATTTCAC
ACCGCATACGTCAAAGCAACCATAGTACGCGCCCTGTAGCGGCGCATTA
AGCGCGGCGGGTGTGGTGGTTACGCGCAGCGTGACCGCTACACTTGCCA
GCGCCCTAGCGCCCGCTCCTTTCGCTTTCTTCCCTTCCTTTCTCGCCAC
GTTCGCCGGCTTTCCCCGTCAAGCTCTAAATCGGGGGCTCCCTTTAGGG
TTCCGATTTAGTGCTTTACGGCACCTCGACCCCAAAAAACTTGATTTGG
GTGATGGTTCACGTAGTGGGCCATCGCCCTGATAGACGGTTTTTCGCCC
TTTGACGTTGGAGTCCACGTTCTTTAATAGTGGACTCTTGTTCCAAACT
GGAACAACACTCAACCCTATCTCGGGCTATTCTTTTGATTTATAAGGGA
TTTTGCCGATTTCGGCCTATTGGTTAAAAAATGAGCTGATTTAACAAAA
ATTTAACGCGAATTTTAACAAAATATTAACGTTTACAATTTTATGGTGC
ACTCTCAGTACAATCTGCTCTGATGCCGCATAGTTAAGCCAGCCCCGAC
ACCCGCCAACACCCGCTGACGCGCCCTGACGGGCTTGTCTGCTCCCGGC
ATCCGCTTACAGACAAGCTGTGACCGTCTCCGGGAGCTGCATGTGTCAG
AGGTTTTCACCGTCATCACCGAAACGCGCGAGACGAAAGGGCCTCGTGA
TACGCCTATTTTTATAGGTTAATGTCATGATAATAATGGTTTCTTAGAC
GTCAGGTGGCACTTTTCGGGGAAATGTGCGCGGAACCCCTATTTGTTTA
TTTTTCTAAATACATTCAAATATGTATCCGCTCATGAGACAATAACCCT
GATAAATGCTTCAATAATATTGAAAAAGGAAGAGTATGAGTATTCAACA
TTTCCGTGTCGCCCTTATTCCCTTTTTTGCGGCATTTTGCCTTCCTGTT
TTTGCTCACCCAGAAACGCTGGTGAAAGTAAAAGATGCTGAAGATCAGT
TGGGTGCACGAGTGGGTTACATCGAACTGGATCTCAACAGCGGTAAGAT
CCTTGAGAGTTTTCGCCCCGAAGAACGTTTTCCAATGATGAGCACTTTT
AAAGTTCTGCTATGTGGCGCGGTATTATCCCGTATTGACGCCGGGCAAG
AGCAACTCGGTCGCCGCATACACTATTCTCAGAATGACTTGGTTGAGTA
CTCACCAGTCACAGAAAAGCATCTTACGGATGGCATGACAGTAAGAGAA
TTATGCAGTGCTGCCATAACCATGAGTGATAACACTGCGGCCAACTTAC
TTCTGACAACGATCGGAGGACCGAAGGAGCTAACCGCTTTTTTGCACAA
CATGGGGGATCATGTAACTCGCCTTGATCGTTGGGAACCGGAGCTGAAT
GAAGCCATACCAAACGACGAGCGTGACACCACGATGCCTGTAGCAATGG
CAACAACGTTGCGCAAACTATTAACTGGCGAACTACTTACTCTAGCTTC
CCGGCAACAATTAATAGACTGGATGGAGGCGGATAAAGTTGCAGGACCA
CTTCTGCGCTCGGCCCTTCCGGCTGGCTGGTTTATTGCTGATAAATCTG
GAGCCGGTGAGCGTGGGTCTCGCGGTATCATTGCAGCACTGGGGCCAGA
TGGTAAGCCCTCCCGTATCGTAGTTATCTACACGACGGGGAGTCAGGCA
ACTATGGATGAACGAAATAGACAGATCGCTGAGATAGGTGCCTCACTGA
TTAAGCATTGGTAACTGTCAGACCAAGTTTACTCATATATACTTTAGAT
TGATTTAAAACTTCATTTTTAATTTAAAAGGATCTAGGTGAAGATCCTT
TTTGATAATCTCATGACCAAAATCCCTTAACGTGAGTTTTCGTTCCACT
GAGCGTCAGACCCCGTAGAAAAGATCAAAGGATCTTCTTGAGATCCTTT
TTTTCTGCGCGTAATCTGCTGCTTGCAAACAAAAAAACCACCGCTACCA
GCGGTGGTTTGTTTGCCGGATCAAGAGCTACCAACTCTTTTTCCGAAGG
TAACTGGCTTCAGCAGAGCGCAGATACCAAATACTGTCCTTCTAGTGTA
GCCGTAGTTAGGCCACCACTTCAAGAACTCTGTAGCACCGCCTACATAC
CTCGCTCTGCTAATCCTGTTACCAGTGGCTGCTGCCAGTGGCGATAAGT
CGTGTCTTACCGGGTTGGACTCAAGACGATAGTTACCGGATAAGGCGCA
GCGGTCGGGCTGAACGGGGGGTTCGTGCACACAGCCCAGCTTGGAGCGA
ACGACCTACACCGAACTGAGATACCTACAGCGTGAGCTATGAGAAAGCG
CCACGCTTCCCGAAGGGAGAAAGGCGGACAGGTATCCGGTAAGCGGCAG
GGTCGGAACAGGAGAGCGCACGAGGGAGCTTCCAGGGGGAAACGCCTGG
TATCTTTATAGTCCTGTCGGGTTTCGCCACCTCTGACTTGAGCGTCGAT
TTTTGTGATGCTCGTCAGGGGGGCGGAGCCTATGGAAAAACGCCAGCAA
CGCGGCCTTTTTACGGTTCCTGGCCTTTTGCTGGCCTTTTGCTCACATG
T

Preferably, the vector comprises a nucleic acid sequence substantially as set out in SEQ ID No: 16, or a fragment or variant thereof.

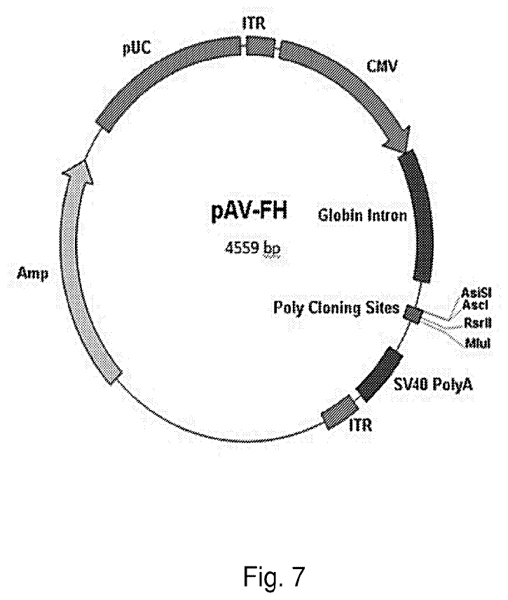

The following sequence, referred to herein as SEQ ID NO: 17, encodes a vector carrying AAV2 right and left ITRs. This vector is suitable for the production of AAV vectors; the genetic constructs of the first aspect can be subcloned into this vector. A map showing the features of a plasmid comprising SEQ ID NO: 17 is shown in FIG. 7. This vector is purely for illustrative purposes, and the skilled person would be aware of other suitable vectors. The pAV-FH vector sequence shown in FIG. 7, and other suitable vectors for the production of AAV vectors, are commercially available.

[SEQ ID NO: 17]
CCTGCAGGCAGCTGCGCGCTCGCTCGCTCACTGAGGCCGCCCGGGCGTC
GGGCGACCTTTGGTCGCCCGGCCTCAGTGAGCGAGCGAGCGCGCAGAGA
GGGAGTGGCCAACTCCATCACTAGGGGTTCCTGCGGCCGGTCGCGTCTA
GTTATTAATAGTAATCAATTACGGGGTCATTAGTTCATAGCCCATATAT
GGAGTTCCGCGTTACATAACTTACGGTAAATGGCCCGCCTGGCTGACCG
CCCAACGACCCCCGCCCATTGACGTCAATAATGACGTATGTTCCCATAG
TAACGCAATAGGGACTTTCCATTGACGTCAATGGGTGGAGTATTTACGG
TAAACTGCCCACTTGGCAGTACATCAAGTGTATCATATGCCAAGTACGC
CCCCTATTGACGTCAATGACGGTAAATGGCCCGCCTGGCATTATGCCCA
GTACATGACCTTATGGGACTTTCCTACTTGGCAGTACATCTACGTATTA
GTCATCGCTATTACCATGGTGATGCGGTTTTGGCAGTACATCAATGGGC
GTGGATAGCGGTTTGACTCACGGGGATTTCCAAGTCTCCACCCCATTGA
CGTCAATGGGAGTTTGTTTTGCACCAAAATCAACGGGACTTTCCAAAAT
GTCGTAACAACTCCGCCCCATTGACGCAAATGGGCGGTAGGCGTGTACG
GTGGGAGGTCTATATAAGCAGAGCTGTTTAGTGAACCGTCAGATCGCCT
GGAGACGCCATCCACGCTGTTTTGACCTCCATAGAAGACACCGGGACCG
ATCCAGCCTCCGCGGATTCGAATCCCGGCCGGGAACGGTGCATTGGAAC
GCGGATTCCCCGTGCCAAGAGTGACGTAAGTACCGCCTATAGAGTCTAT
AGGCCCACAAAAAATGCTTTCTTCTTTTAATATACTTTTTTGTTTATCT
TATTTCTAATACTTTCCCTAATCTCTTTCTTTCAGGGCAATAATGATAC
AATGTATCATGCCTCTTTGCACCATTCTAAAGAATAACAGTGATAATTT
CTGGGTTAAGGCAATAGCAATATTTCTGCATATAAATATTTCTGCATAT
AAATTGTAACTGATGTAAGAGGTTTCATATTGCTAATAGCAGCTACAAT
CCAGCTACCATTCTGCTTTTATTTTATGGTTGGGATAAGGCTGGATTAT
TCTGAGTCCAAGCTAGGCCCTTTTGCTAATCATGTTCATACCTCTTATC
TTCCTCCCACAGCTCCTGGGCAACGTGCTGGTCTGTGTGCTGGCCCATC
ACTTTGGCAAAGAATTGGGATTCGAACATCGATTGAATTCAGATCCGCT
AGTAATACGACTCACTATAGGGAGAGGATCCGGTACCGAGGAGATCTGC
CGCCGCGATCGCCGGCGCGCCAGATCTCACGCTTAACTAGCTAGCGGAC
CGACGCGTACGCGGCCGCTCGAGGATTATAAGGATGACGACGATAAATT
CGTCGAGCACCACCACCACCACCACTAATAAGGTTTATCCGATCCACCG
GATCTAGATAAGATATCCGATCCACCGGATCTAGATAACTGATCATAAT
CAGCCATACCACATTTGTAGAGGTTTTACTTGCTTTAAAAAACCTCCCA
CACCTCCCCCTGAACCTGAAACATAAAATGAATGCAATTGTTGTTGTTA
ACTTGTTTATTGCAGCTTATAATGGTTACAAATAAAGCAATAGCATCAC
AAATTTCACAAATAAAGCATTTTTTTCACTGCATTCTAGTTGTGGTTTG
TCCAAACTCATCAATGTATCTTAACGCGGTAACCACGTGCGGACCCAAC
GGCCGCAGGAACCCCTAGTGATGGAGTTGGCCACTCCCTCTagagCTGC
GCGCTCGCTCGCTCACTGAGGCCGGGCGACCAAAGGTCGCCCGACGCCC
GGGCTTTGCCCGGGCGGCCTCAGTGAGCGAGCGAGCGCGCAGCTGCCTG
CAGGGGCGCCTGATGCGGTATTTTCTCCTTACGCATCTGTGCGGTATTT
CACACCGCATACGTCAAAGCAACCATAGTACGCGCCCTGTAGCGGCGCA
TTAAGCGCGGCGGGTGTGGTGGTTACGCGCAGCGTGACCGCTACACTTG
CCAGCGCCCTAGCGCCCGCTCCTTTCGCTTTCTTCCCTTCCTTTCTCGC
CACGTTCGCCGGCTTTCCCCGTCAAGCTCTAAATCGGGGGCTCCCTTTA
GGGTTCCGATTTAGTGCTTTACGGCACCTCGACCCCAAAAAACTTGATT
TGGGTGATGGTTCACGTAGTGGGCCATCGCCCTGATAGACGGTTTTTCG
CCCTTTGACGTTGGAGTCCACGTTCTTTAATAGTGGACTCTTGTTCCAA
ACTGGAACAACACTCAACCCTATCTCGGGCTATTCTTTTGATTTATAAG
GGATTTTGCCGATTTCGGCCTATTGGTTAAAAAATGAGCTGATTTAACA
AAAATTTAACGCGAATTTTAACAAAATATTAACGTTTACAATTTTATGG
TGCACTCTCAGTACAATCTGCTCTGATGCCGCATAGTTAAGCCAGCCCC
GACACCCGCCAACACCCGCTGACGCGCCCTGACGGGCTTGTCTGCTCCC
GGCATCCGCTTACAGACAAGCTGTGACCGTCTCCGGGAGCTGCATGTGT
CAGAGGTTTTCACCGTCATCACCGAAACGCGCGAGACGAAAGGGCCTCG
TGATACGCCTATTTTTATAGGTTAATGTCATGATAATAATGGTTTCTTA
GACGTCAGGTGGCACTTTTCGGGGAAATGTGCGCGGAACCCCTATTTGT
TTATTTTTCTAAATACATTCAAATATGTATCCGCTCATGAGACAATAAC
CCTGATAAATGCTTCAATAATATTGAAAAAGGAAGAGTATGAGTATTCA
ACATTTCCGTGTCGCCCTTATTCCCTTTTTTGCGGCATTTTGCCTTCCT
GTTTTTGCTCACCCAGAAACGCTGGTGAAAGTAAAAGATGCTGAAGATC
AGTTGGGTGCACGAGTGGGTTACATCGAACTGGATCTCAACAGCGGTAA
GATCCTTGAGAGTTTTCGCCCCGAAGAACGTTTTCCAATGATGAGCACT
TTTAAAGTTCTGCTATGTGGCGCGGTATTATCCCGTATTGACGCCGGGC
AAGAGCAACTCGGTCGCCGCATACACTATTCTCAGAATGACTTGGTTGA
GTACTCACCAGTCACAGAAAAGCATCTTACGGATGGCATGACAGTAAGA
GAATTATGCAGTGCTGCCATAACCATGAGTGATAACACTGCGGCCAACT
TACTTCTGACAACGATCGGAGGACCGAAGGAGCTAACCGCTTTTTTGCA
CAACATGGGGGATCATGTAACTCGCCTTGATCGTTGGGAACCGGAGCTG
AATGAAGCCATACCAAACGACGAGCGTGACACCACGATGCCTGTAGCAA
TGGCAACAACGTTGCGCAAACTATTAACTGGCGAACTACTTACTCTAGC
TTCCCGGCAACAATTAATAGACTGGATGGAGGCGGATAAAGTTGCAGGA
CCACTTCTGCGCTCGGCCCTTCCGGCTGGCTGGTTTATTGCTGATAAAT
CTGGAGCCGGTGAGCGTGGGTCTCGCGGTATCATTGCAGCACTGGGGCC
AGATGGTAAGCCCTCCCGTATCGTAGTTATCTACACGACGGGGAGTCAG
GCAACTATGGATGAACGAAATAGACAGATCGCTGAGATAGGTGCCTCAC
TGATTAAGCATTGGTAACTGTCAGACCAAGTTTACTCATATATACTTTA
GATTGATTTAAAACTTCATTTTTAATTTAAAAGGATCTAGGTGAAGATC
CTTTTTGATAATCTCATGACCAAAATCCCTTAACGTGAGTTTTCGTTCC
ACTGAGCGTCAGACCCCGTAGAAAAGATCAAAGGATCTTCTTGAGATCC
TTTTTTTCTGCGCGTAATCTGCTGCTTGCAAACAAAAAAACCACCGCTA
CCAGCGGTGGTTTGTTTGCCGGATCAAGAGCTACCAACTCTTTTTCCGA
AGGTAACTGGCTTCAGCAGAGCGCAGATACCAAATACTGTCCTTCTAGT
GTAGCCGTAGTTAGGCCACCACTTCAAGAACTCTGTAGCACCGCCTACA
TACCTCGCTCTGCTAATCCTGTTACCAGTGGCTGCTGCCAGTGGCGATA
AGTCGTGTCTTACCGGGTTGGACTCAAGACGATAGTTACCGGATAAGGC
GCAGCGGTCGGGCTGAACGGGGGGTTCGTGCACACAGCCCAGCTTGGAG
CGAACGACCTACACCGAACTGAGATACCTACAGCGTGAGCTATGAGAAA
GCGCCACGCTTCCCGAAGGGAGAAAGGCGGACAGGTATCCGGTAAGCGG
CAGGGTCGGAACAGGAGAGCGCACGAGGGAGCTTCCAGGGGGAAACGCC
TGGTATCTTTATAGTCCTGTCGGGTTTCGCCACCTCTGACTTGAGCGTC
GATTTTTGTGATGCTCGTCAGGGGGGCGGAGCCTATGGAAAAACGCCAG
CAACGCGGCCTTTTTACGGTTCCTGGCCTTTTGCTGGCCTTTTGCTCAC
ATGT

Preferably, the recombinant vector of the invention may comprise a nucleic acid sequence which enhances expression of tyrosine hydroxylase (TH) and GTP cyclohydrolase 1 (GCH1). More preferably, the nucleic acid sequence comprises or consists of an optimised intron with pUC origin and RNA-OUT (OIPR) sequence, as described in Lu et al, 2017, “A 5′ Noncoding Exon Containing Engineered Intron Enhances Transgene Expression from Recombinant AAV Vectors in vivo”, Human Gene Therapy, Volume 28, Page 125-134 and WO2013119371.

The OIPR sequence may be referred to herein as SEQ ID No: 26, as follows:

[SEQ ID No: 26]
ATTGGGATCTTCACACAGCAGGTAAGGTTGCGGGCCGGGCCTGGGCCGG
GTCCGGGCCGGGTATTGCCCGCCTAATGAGCGGGCTTTTTTTTCTTACC
CCTTCTTCCGCTTCCTCGCTCACTGACTCGCTGCGCTCGGTCGTTCGGC
TGCGGCGAGCGGTATCAGCTCACTCAAAGGCGGTAATACGGTTATCCAC
AGAATCAGGGGATAACGCAGGAAAGAACATGTGAGCAAAAGGCCAGCAA
AAGGCCAGGAACCGTAAAAAGGCCGCGTTGCTGGCGTTTTTCCATAGGC
TCCGCCCCCCTGACGAGCATCACAAAAATCGACGCTCAAGTCAGAGGTG
GCGAAACCCGACAGGACTATAAAGATACCAGGCGTTTCCCCCTGGAAGC
TCCCTCGTGCGCTCTCCTGTTCCGACCCTGCCGCTTACCGGATACCTGT
CCGCCTTTCTCCCTTCGGGAAGCGTGGCGCTTTCTCATAGCTCACGCTG
TAGGTATCTCAGTTCGGTGTAGGTCGTTCGCTCCAAGCTGGGCTGTGTG
CACGAACCCCCCGTTCAGCCCGACCGCTGCGCCTTATCCGGTAACTATC
GTCTTGAGTCCAACCCGGTAAGACACGACTTATCGCCACTGGCAGCAGC
CACTGGTAACAGGATTAGCAGAGCGAGGTATGTAGGCGGTGCTACAGAG
TTCTTGAAGTGGTGGCCTAACTACGGCTACACTAGAAGAACAGTATTTG
GTATCTGCGCTCTGCTGAAGCCAGTTACCTTCGGAAAAAGAGTTGGTAG
CTCTTGATCCGGCAAACAAACCACCGCTGGTAGCGGTGGTTTTTTTGTT
TGCAAGCAGCAGATTACGCGCAGAAAAAAAGGATCTCAAGAAGATCCTT
TGATCTTTTCTACGGGGTCTGACGCTCAGTGGAACGAAAACTCACGTTA
AGGGATTTTGGTCATGAGATTATCAAAAAGGATCTTCACCTAGATCCTT
TTAAATTAAAAATGAAGTTTTAAATCAATCTAAAGTATATATGAGTAAA
CTTGGTCTGACAGTTACCAATGCTTAATCAGTGAGGCACCTATCTCAGC
GATCTGTCTATTTCGTTCATCCATAGTTGCCTGACTCCTGCAAACCACG
TTGTGGTAGAATTGGTAAAGAGAGTCGTGTAAAATATCGAGTTCGCACA
TCTTGTTGTCTGATTATTGATTTTTGGCGAAACCATTTGATCATATGAC
AAGATGTGTATCTACCTTAACTTAATGATTTTGATAAAAATCATTAGGT
ACCCCGGCCCGCACTGACCCCTGGTGTTGCTTTTTTTTTTTAGGCCGCA
AGCTGAAGCGTGTCC

Preferably, the OIPR sequence comprises a nucleic acid sequence substantially as set out in SEQ ID No: 26, or a fragment or variant thereof.

Preferably, the OIPR sequence is located within the main cassette and is disposed 3′ of the promoter sequence, and 5′ of the coding sequences of tyrosine hydroxylase (TH) and GTP cyclohydrolase 1 (GCH1).

The gene therapy vectors may be produced by any technique known in the art. For instance, the rAAV vectors may be produced using classic triple transfection methodology. Methods for the production of adeno-associated virus vectors are disclosed in Matsushita et al. (Matsushita et al., Adeno-associated virus vectors can be efficiently produced without helper virus. Gene Therapy (1998) 5, 938-945)

Hence, according to a third aspect, there is provided a method of manufacturing a gene therapy vector, the method comprising, (i) introducing into a host cell a nucleic acid sequence comprising a genetic construct according to the first aspect or a recombinant vector according to the second aspect, and (ii) culturing the host cell under conditions to result in the production of a gene therapy vector comprising the genetic construct according to the first aspect or the recombinant vector according to the second aspect.

The recombinant vectors of the second aspect are particularly suitable for therapy.

Hence, according to a fourth aspect, there is provided the genetic construct according to the first aspect, or the recombinant vector according to the second aspect, for use as a medicament or in therapy.

According to a fifth aspect, there is provided the genetic construct according to the first aspect, or the recombinant vector according to the second aspect, for use in treating, preventing, or ameliorating a neurodegenerative disorder.

According to a sixth aspect, there is provided a method of treating, preventing, or ameliorating a neurodegenerative disorder in a subject, the method comprising administering, to a subject in need of such treatment, a therapeutically effective amount of the genetic construct according to the first aspect, or the recombinant vector according to the second aspect.

Preferably, the genetic construct or the recombinant vector according to the invention are used in a gene therapy technique.

In an embodiment, the neurodegenerative disorder to be treated is a disease associated with catecholamine dysfunction. In a preferred embodiment, the catecholamine dysfunction may be characterised by a dopamine deficiency. In another embodiment, the disorder to be treated is selected from the group consisting of Parkinson's disease, DOPA responsive dystonia, vascular parkinsonism, side effects associated with L-DOPA treatment, L-DOPA induced dyskinesia, Segawa syndrome, or genetic dopamine receptor abnormalities. In a more preferred embodiment, the disease to be treated is Parkinson's disease.

The disclosed gene therapy technique leads to a constant level of production of L-DOPA in the striatum. This removes or reduces the need for oral L-DOPA and so results in reduced peak to trough variation. Hence, the disclosed gene therapy can be used for the treatment of side effects associated with L-DOPA treatment and of L-DOPA induced dyskinesia.

The disclosed gene therapy technique may be used for the treatment of Segawa syndrome. Although it would be possible to treat Segawa syndrome with a gene therapy delivering only GCH1, the inclusion of TH is not expected to be prejudicial and may be beneficial. The disclosed treatment is especially advantageous as, due to the rareness of Segawa syndrome, it may not be commercially attractive or viable to develop a treatment solely for this indication. Production of the disclosed invention for this indication as well as for other neurodegenerative diseases, such as Parkinson's disease, will reduce the unit cost of the therapy.

In a preferred embodiment, medicaments according to the invention may be administered to a subject by injection into the blood stream, a nerve, or directly into a site requiring treatment. For instance, the vector may be delivered to the brain. Specific regions of the brain may be targeted, such as striatum. The putamen or caudate nucleus may be targeted. The treatment may be centred on the dopaminergic neurons of the pars compacta region in the substantia nigra.

The delivery method may be direct injection. Methods for injection into the brain (for instance the striatum) are well known in the art (Bilang-Bleuel et al (1997) Proc. Acad. Natl. Sci. USA 94:8818-8823; Choi-Lundberg et al (1998) Exp. Neurol. 154:261-275; Choi-Lundberg et al (1997) Science 275:838-841; and Mandel et al (1997)) Proc. Acad. Natl. Sci. USA 94:14083-14088). Alternatively, or in addition, the vector chosen may have a tropism that is targeted towards a specific desired tissue, such as a neurone.

Modifications of the vector capsid properties could enable targeting of the vector to the striatal region also after intrathecal (IT) injection or injection into the cerebral ventricles (ICV). An alternative approach is to generate chimeric AAV serotypes that would inherit different binding properties from the two serotypes mixed.

In one embodiment the genome sequence described herein, i.e. the promoter-TH-linker-GCH1 sequence, may be administered by injection directly as naked DNA without a viral vector. The naked DNA may be administered as a plasmid. The naked DNA may be delivered as a plasmid administered in any suitable non-viral carrier that would be known to those skilled in the art.

Preferably, the non-viral carrier is selected from the groups consisting of: poly(2-ethyl-2-oxazoline)-PLA-g-PEI amphiphilic triblock micelles, a Poly(β-amino ester)-based biodegradable nanoparticle, a Pluronic® block-copolymer such as Pluronic F27, Pluronic F68 or Pluronic F85 a mixture of Pluronics such as SP1017, and a carrier such as BrainFectIn® (OZ Biosciences, Marseille, France).

It will be appreciated that the amount of the genetic construct or the recombinant vector that is required is determined by its biological activity and bioavailability, which in turn depends on the mode of administration, the physiochemical properties of the genetic construct or the recombinant vector and whether it is being used as a monotherapy or in a combined therapy. Optimal dosages to be administered may be determined by those skilled in the art, and will vary with the particular genetic construct or the recombinant vector in use, the strength of the pharmaceutical composition, the mode of administration, and the advancement of the neurodegenerative disorder. Additional factors depending on the particular subject being treated will result in a need to adjust dosages, including subject age, weight, gender, diet, and time of administration.

The dose delivered may be 300 μl to 4500 μl, 400 μl to 4000 μl, 500 μl to 3500 μl, 600 μl to 3000 μl, 700 μl to 2500 μl, 750 μl to 2000 μl, 800 μl to 1500 μl, 850 μl to 1000 μl, or roughly 900 μl. For delivery methods involving direct injection into the striatum, the two doses may be delivered, one per striatum. For example, one dose, such as 900 μl, may be delivered to the left side of the brain, and a further dose, such as 900 μl, may be delivered to the right side of the brain.

The titre of the dose may be 1E8 to 5E14, 1E9 to 1E14, 1E10 to 5E13, 1E11 to 1E13, 1E12 to 8E12, 4E12 to 6E12, or roughly 5E12 genome copies per ml (GC/ml).

The genetic construct or the recombinant vector may be administered before, during or after onset of the disorder. Doses may be given as a single administration, or multiple doses may be given over the course of the treatment. A dose may be administered to a patient, and the patient may be monitored in order to assess the necessity for a second or further doses. Repeat use delivery of the same genome may be facilitated by the switching the AAV capsid serotype to reduce the probability of interference by an antibody or cell mediated immune response induced by the previous treatment.

In some embodiments, the therapeutic methods may include, prior to gene therapy treatment, a test infusion of L-DOPA. The test infusion may be used to demonstrate that a subject is responsive to L-DOPA, and so may allow the selection of subjects most likely to benefit from gene therapy treatment. The L-DOPA test infusion may be by any means capable of creating a steady blood level over hours or days. Examples of suitable infusion methods include by nasogastric tube, i.v. infusion, infusion via a pump, by the use of DuoDOPA, or any other suitable means.

It will be appreciated that the genetic construct according to the first aspect, or the recombinant vector according to the second aspect may be used in a medicament, which may be used as a monotherapy (i.e. use of the genetic construct according to the first aspect or the vector according to the second aspect of the invention), for treating, ameliorating, or preventing any disorder as disclosed herein. Alternatively, the genetic construct or the recombinant vector according to the invention may be used as an adjunct to, or in combination with, known therapies for treating, ameliorating, or preventing any disorder as disclosed herein. In some cases, the genetic construct may be used as an adjunct to, in combination with, or alongside a treatment designed to improve the gene therapy. For instance, the genetic construct may be used in combination with an immunosuppressive treatment, in order to reduce, prevent, or control an immune response induced by the gene therapy itself. For example, the immunosuppressive treatment may prevent, reduce, or control an immune response directed to a capsid of a gene therapy vector, a genome comprised within a gene therapy vector, or a product produced by a gene therapy vector during therapy. The immunosuppressive regime may include a general immunosuppressant, such as steroid. The immunosuppressive regime may include more targeted immunosuppression designed to reduce specific immune responses, such as immunotherapy to specific antigens found within, or produced by, a gene therapy construct.

The genetic construct according or the recombinant vector according to the invention may be combined in compositions having a number of different forms depending, in particular, on the manner in which the composition is to be used. Thus, for example, the composition may be in the form of a powder, liquid, aerosol, spray, micellar solution, liposome suspension or any other suitable form that may be administered to a person or animal in need of treatment. It will be appreciated that the vehicle of medicaments according to the invention should be one which is well-tolerated by the subject to whom it is given.

Known procedures, such as those conventionally employed by the pharmaceutical industry (e.g. in vivo experimentation, clinical trials, etc.), may be used to form specific formulations of the genetic construct or the recombinant vector according to the invention and precise therapeutic regimes.

According to a seventh aspect, there is provided a pharmaceutical composition comprising the genetic construct according to the first aspect, or the recombinant vector according to the second aspect, and a pharmaceutically acceptable vehicle.

According to an eighth aspect, there is provided a method of preparing the pharmaceutical composition according to the seventh aspect, the method comprising contacting the genetic construct according to the first aspect, or the recombinant vector according to the second aspect, with a pharmaceutically acceptable vehicle.

A “subject” may be a vertebrate, mammal, or domestic animal. Hence, compositions and medicaments according to the invention may be used to treat any mammal, for example livestock (e.g. a horse), pets, or may be used in other veterinary applications. Most preferably, however, the subject is a human being.

A “therapeutically effective amount” of the genetic construct, the recombinant vector or the pharmaceutical composition is any amount which, when administered to a subject, is the amount of the aforementioned that is needed to treat the neurodegenerative disorder.

For example, the therapeutically effective amount of the genetic construct, the recombinant vector or the pharmaceutical composition used may be from about 0.01 mg to about 800 mg, and preferably from about 0.01 mg to about 500 mg. It is preferred that the amount of the genetic construct, the recombinant vector or the pharmaceutical composition is an amount from about 0.1 mg to about 250 mg, and most preferably from about 0.1 mg to about 20 mg.

A “pharmaceutically acceptable vehicle” as referred to herein, is any known compound or combination of known compounds that are known to those skilled in the art to be useful in formulating pharmaceutical compositions.

In a preferred embodiment, the pharmaceutically acceptable vehicle may be such as to allow injection of the composition directly into a subject. For instance, the vehicle may be suitable for allowing the injection of the composition into the brain.

In one embodiment, the pharmaceutically acceptable vehicle may be a solid, and the composition may be in the form of a powder, or suspension. A solid pharmaceutically acceptable vehicle may include one or more substances which may also act as, lubricants, solubilisers, suspending agents, dyes, fillers, glidants, compression aids, inert binders, preservatives, dyes, coatings, or solid-disintegrating agents. The vehicle may also be an encapsulating material. In powders, the vehicle is a finely divided solid that is in admixture with the finely divided active agents according to the invention. In another embodiment, the pharmaceutical vehicle may be a gel or the like.

However, the pharmaceutical vehicle may be a suspension or a liquid, and the pharmaceutical composition is in the form of a suspension or a solution. Liquid pharmaceutical compositions, which are sterile solutions or suspensions, can be utilized by, for example, intramuscular, intrathecal, epidural, intraperitoneal, intravenous and particularly subcutaneous injection. The genetic construct or the recombinant vector may be prepared as a sterile solid composition that may be dissolved or suspended at the time of administration using sterile water, saline, Dulbecco's Phosphate Buffered Saline (dPBS) with MgCl₂ and CaCl₂), or other appropriate sterile injectable medium.

Finally, in another aspect of the invention, there is provided a genetic construct comprising a promoter operably linked to a first coding sequence, which encodes tyrosine hydroxylase (TH), and a second coding sequence, which encodes GTP cyclohydrolase 1 (GCH1), wherein the second coding sequence is 3′ to the first coding sequence, and the first and second coding sequences are part of a single operon.

It will be appreciated that the invention extends to any nucleic acid or peptide or variant, derivative or analogue thereof, which comprises substantially the amino acid or nucleic acid sequences of any of the sequences referred to herein, including variants or fragments thereof. The terms “substantially the amino acid/nucleotide/peptide sequence”, “variant” and “fragment”, can be a sequence that has at least 40% sequence identity with the amino acid/nucleotide/peptide sequences of any one of the sequences referred to herein, for example 40% identity with the sequence identified as SEQ ID No: 1-31, and so on.

Amino acid/polynucleotide/polypeptide sequences with a sequence identity which is greater than 65%, more preferably greater than 70%, even more preferably greater than 75%, and still more preferably greater than 80% sequence identity to any of the sequences referred to are also envisaged. Preferably, the amino acid/polynucleotide/polypeptide sequence has at least 85% identity with any of the sequences referred to, more preferably at least 90% identity, even more preferably at least 92% identity, even more preferably at least 95% identity, even more preferably at least 97% identity, even more preferably at least 98% identity and, most preferably at least 99% identity with any of the sequences referred to herein.

The skilled technician will appreciate how to calculate the percentage identity between two amino acid/polynucleotide/polypeptide sequences. In order to calculate the percentage identity between two amino acid/polynucleotide/polypeptide sequences, an alignment of the two sequences must first be prepared, followed by calculation of the sequence identity value. The percentage identity for two sequences may take different values depending on:—(i) the method used to align the sequences, for example, ClustalW, BLAST, FASTA, Smith-Waterman (implemented in different programs), or structural alignment from 3D comparison; and (ii) the parameters used by the alignment method, for example, local vs global alignment, the pair-score matrix used (e.g. BLOSUM62, PAM250, Gonnet etc.), and gap-penalty, e.g. functional form and constants.

Having made the alignment, there are many different ways of calculating percentage identity between the two sequences. For example, one may divide the number of identities by: (i) the length of shortest sequence; (ii) the length of alignment; (iii) the mean length of sequence; (iv) the number of non-gap positions; or (v) the number of equivalenced positions excluding overhangs. Furthermore, it will be appreciated that percentage identity is also strongly length dependent. Therefore, the shorter a pair of sequences is, the higher the sequence identity one may expect to occur by chance.

Hence, it will be appreciated that the accurate alignment of protein or DNA sequences is a complex process. The popular multiple alignment program ClustalW (Thompson et al., 1994, Nucleic Acids Research, 22, 4673-4680; Thompson et al., 1997, Nucleic Acids Research, 24, 4876-4882) is a preferred way for generating multiple alignments of proteins or DNA in accordance with the invention. Suitable parameters for ClustalW may be as follows: For DNA alignments: Gap Open Penalty=15.0, Gap Extension Penalty=6.66, and Matrix=Identity. For protein alignments: Gap Open Penalty=10.0, Gap Extension Penalty=0.2, and Matrix=Gonnet. For DNA and Protein alignments: ENDGAP=−1, and GAPDIST=4. Those skilled in the art will be aware that it may be necessary to vary these and other parameters for optimal sequence alignment.

Preferably, calculation of percentage identities between two amino acid/polynucleotide/polypeptide sequences may then be calculated from such an alignment as (N/T)*100, where N is the number of positions at which the sequences share an identical residue, and T is the total number of positions compared including gaps but excluding overhangs. Hence, a most preferred method for calculating percentage identity between two sequences comprises (i) preparing a sequence alignment using the ClustalW program using a suitable set of parameters, for example, as set out above; and (ii) inserting the values of N and T into the following formula:—Sequence Identity=(N/T)*100.

Alternative methods for identifying similar sequences will be known to those skilled in the art. For example, a substantially similar nucleotide sequence will be encoded by a sequence which hybridizes to DNA sequences or their complements under stringent conditions. By stringent conditions, we mean the nucleotide hybridises to filter-bound DNA or RNA in 3× sodium chloride/sodium citrate (SSC) at approximately 45° C. followed by at least one wash in 0.2×SSC/0.1% SDS at approximately 20-65° C. Alternatively, a substantially similar polypeptide may differ by at least 1, but less than 5, 10, 20, 50 or 100 amino acids from the sequences shown in, for example, SEQ ID Nos: 3 and 5.

Due to the degeneracy of the genetic code, it is clear that any nucleic acid sequence described herein could be varied or changed without substantially affecting the sequence of the protein encoded thereby, to provide a functional variant thereof. Suitable nucleotide variants are those having a sequence altered by the substitution of different codons that encode the same amino acid within the sequence, thus producing a silent change. Other suitable variants are those having homologous nucleotide sequences but comprising all, or portions of, sequence, which are altered by the substitution of different codons that encode an amino acid with a side chain of similar biophysical properties to the amino acid it substitutes, to produce a conservative change. For example, small non-polar, hydrophobic amino acids include glycine, alanine, leucine, isoleucine, valine, proline, and methionine. Large non-polar, hydrophobic amino acids include phenylalanine, tryptophan and tyrosine. The polar neutral amino acids include serine, threonine, cysteine, asparagine and glutamine. The positively charged (basic) amino acids include lysine, arginine and histidine. The negatively charged (acidic) amino acids include aspartic acid and glutamic acid. It will therefore be appreciated which amino acids may be replaced with an amino acid having similar biophysical properties, and the skilled technician will know the nucleotide sequences encoding these amino acids.

All of the features described herein (including any accompanying claims, abstract and drawings), and/or all of the steps of any method or process so disclosed, may be combined with any of the above aspects in any combination, except combinations where at least some of such features and/or steps are mutually exclusive.

For a better understanding of the invention, and to show how embodiments of the same may be carried into effect, reference will now be made, by way of example, to the accompanying Figure, in which:—

FIG. 1 is a graphical representation of the results of an in vitro assay comparing the TH expression between various embodiments of the constructs of the invention and those described in prior art references;

FIG. 2 is a graphical representation of the results of an in vitro assay comparing the GCH1 expression between various embodiments of the construct of the invention and those described in prior art references;

FIG. 3 is a plasmid map of a first embodiment of the construct of the invention, showing the features of SEQ ID NO: 13;

FIG. 4 is a plasmid map of a second embodiment of the construct of the invention, showing the features of SEQ ID NO: 14;

FIG. 5 is a plasmid map of a third embodiment of the construct of the invention, showing the features of SEQ ID NO: 15;

FIG. 6 is a plasmid map of a fourth embodiment of the construct of the invention, showing the features of SEQ ID NO: 16;

FIG. 7 is a plasmid map of a fifth embodiment of the construct of the invention, showing the features of SEQ ID NO: 17;

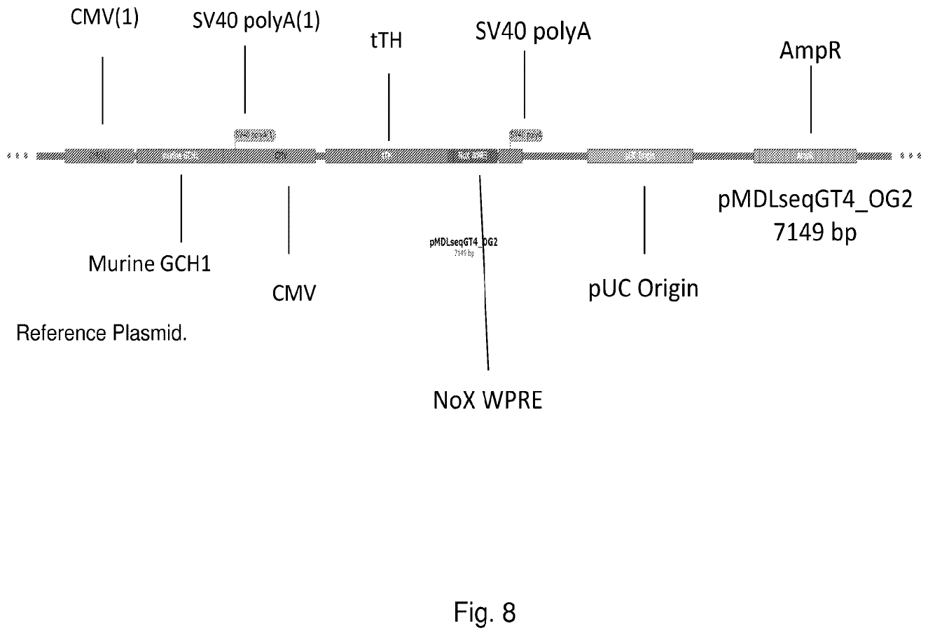

FIG. 8 is a plasmid map of a construct corresponding to the construct of WO2011/054976, and used herein as a reference plasmid;

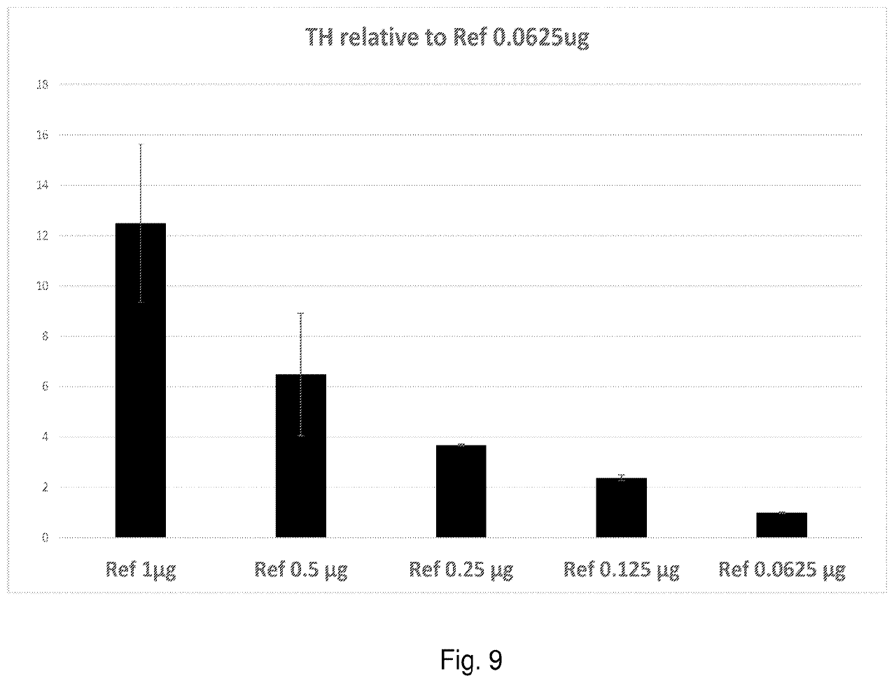

FIG. 9 is a graphical representation of the results of an in vitro assay comparing TH expression at various amounts of DNA;

FIG. 10 is a plasmid map of a sixth embodiment of the construct of the invention, showing the features of SEQ ID NO: 18;

FIG. 11 is a plasmid map of a seventh embodiment of the construct of the invention, showing the features of SEQ ID NO: 19;

FIG. 12 is a plasmid map of an eighth embodiment of the construct of the invention, showing the features of SEQ ID NO: 20;

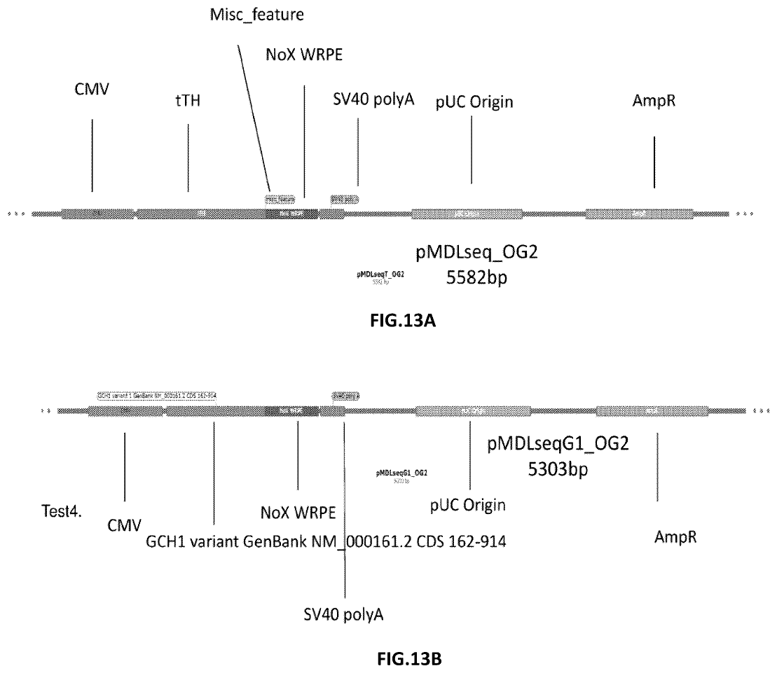

FIG. 13A is a plasmid map of a monocistronic construct which includes TH;

FIG. 13B is a plasmid map of a monocistronic construct which includes GCH1;

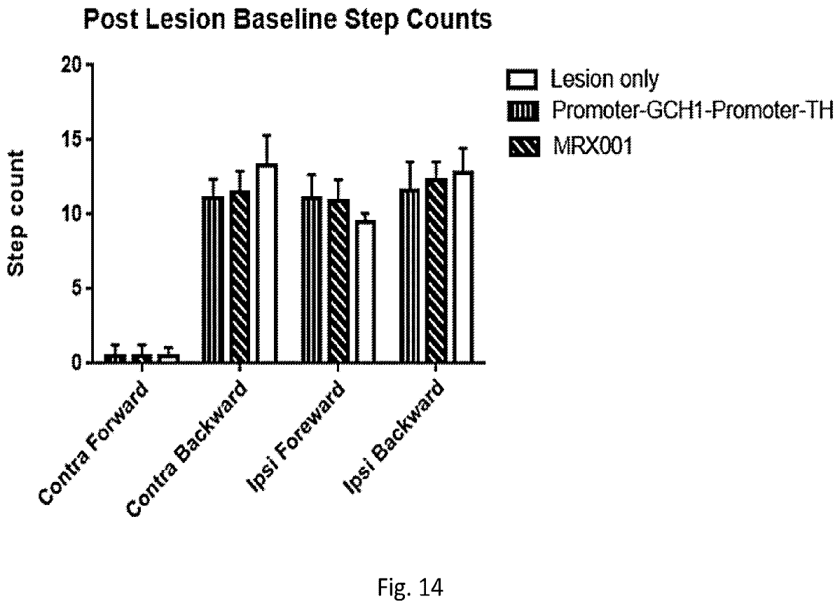

FIG. 14 is a graphical representation of the in vivo stepping assay control experiment showing baseline stepping in rats post-lesion; and

FIG. 15 is a graphical representation of the in vivo stepping assay showing a step count improvement in rats treated with a construct of the invention, when compared to a prior art construct, 4 weeks post treatment.

### EXAMPLES

The inventor has investigated gene therapy for treating neurodegenerative disorders, including dopamine deficiency disorders, and Parkinson's disease. The inventor has developed a novel genetic construct and associated AAV vector for use in treating Parkinson's disease.

### Example 1—Assessment of Tyrosine Hydroxylase Gene Expression

### Background

A reference plasmid corresponding to the prior art plasmid of WO 2011/054976 A2 was utilised in this study. This plasmid is graphically depicted in the map of FIG. 8.

The reference plasmid contains two separate expression cassettes for the murine GTH cyclohydrase 1 (GCH1) and human tyrosine hydroxylase (TH) genes. The purpose of the study was to compare TH expression of the aforementioned reference plasmid at the mRNA level versus three embodiments of the present invention, or a combination of two plasmids, each expressing a single gene, so as to identify a dual gene expression plasmid with improved TH expression.

### Preliminary Experiment

HEK 293 cells were transfected with five different amounts of the reference plasmid, followed by RNA extraction, cDNA synthesis, and subsequent assessment of mRNA expression of GAPDH (housekeeping gene) and human TH by qPCR. The results of this assay are shown in FIG. 9.

Three DNA amounts, 0.25 μg, 0.125 μg and 0.0625 μg, were selected for use in further experiments.

Comparison between the prior art constructs and embodiments of the invention HEK 293 cells were transfected, at the three DNA doses selected from the preliminary experiment, with either the reference plasmid or the four test conditions (Test1-Test4). This was followed by RNA extractions, cDNA synthesis and subsequent assessment of mRNA expression of GAPDH (housekeeping gene) and human tyrosine kinase by qPCR. The sample Ref 0.0625 μg was selected as the calibrator sample for qPCR analysis.

Test1 comprised transfection with a plasmid of a sequence according to SEQ ID NO: 18. This plasmid is graphically depicted in the map of FIG. 10. This construct comprises a sequence encoding TH, an IRES, and a downstream sequence encoding GCH1.

Test2 comprised transfection with a plasmid of a sequence according to SEQ ID NO: 19. This plasmid is graphically depicted in the map of FIG. 11. This construct comprises a sequence encoding TH, a furin cleavage site, a viral 2A peptide spacer, and a downstream sequence encoding GCH1.

Test3 comprised transfection with a plasmid of a sequence according to SEQ ID NO: 20. This plasmid is graphically depicted in the map of FIG. 12. This construct comprises a sequence encoding TH, a flexible linker, and a downstream sequence encoding GCH1.

Test4 comprised transfection involving co-administration of a plasmid graphically depicted in the map of FIG. 13A and of a plasmid graphically depicted in the map of FIG. 13B. One construct encodes TH (FIG. 13A), whereas the other construct encodes GCH1 (FIG. 13B). Test4 was to allow the testing of the expression achieved by the above bicistronic constructs over these two co-administered monocistronic constructs.

The results of this experiment are shown in FIG. 1 and Table 1. Test1, Test2, and Test3 (embodiments of the present invention) were all improved over the reference plasmid. Test1 resulted in an up to 9.4-fold increase in mRNA expression over the reference plasmid. It is notable that if sample Ref 0.0625 μg were used as the calibrator sample, up to 9.9-fold increase in mRNA level could be detected.

TABLE 1
Assessment of Tyrosine Hydroxylase gene expression

| | Av | SD |
|---|---|---|
| Ref 0.25 μg | 1 | 0.032029 |
| Ref 0.125 μg | 0.481388 | 0.024329 |
| Ref 0.0625 μg | 0.261409 | 0.033758 |
| Test1 0.25 μg | 9.411235 | 0.182147 |
| Test1 0.125 μg | 4.417566 | 0.702172 |
| Test1 0.0625 μg | 2.587243 | 0.722136 |
| Test2 0.25 μg | 4.161938 | 2.724108 |
| Test2 0.125 μg | 3.838677 | 2.227054 |
| Test2 0.0625 μg | 1.415666 | 0.075599 |
| Test3 0.25 μg | 2.553369 | 0.039338 |
| Test3 0.125 μg | 1.326091 | 0.157098 |
| Test3 0.0625 μg | 0.574204 | 0.077967 |
| Test4 0.25 μg | 2.877203 | 0.380339 |
| Test4 0.125 μg | 1.401549 | 0.002572 |
| Test4 0.0625 μg | 1.009721 | 0.216553 |

Surprisingly, Test1 and Test2 were also improved over Test4 (the co-administered monocistronic constructs). This is particularly advantageous as therapy with a single biscistronic construct has further advantages over therapy using two monocistronic constructs. These advantages are as disclosed herein, but in short the bicistronic construct ensures that the genes are delivered to the same cell and also has advantages in manufacturing and economy.

### Example 2—Assessment of Murine GTP Cyclohydrolase 1 Gene Expression

The purpose of this study was to quantify expression of the murine GCH1 gene from vectors featuring a number of gene configurations. In particular, two embodiments of the invention were compared to a reference plasmid corresponding to the prior art plasmid of WO 2011/054976 A2.

The reference plasmid was the same as that described in Example 1 and FIG. 8.

The plasmid used in Test1 was the same as that described in Example 1, FIG. 10, and SEQ ID NO: 18.

The plasmid used in Test2 was the same as that described in Example 1, FIG. 11, and SEQ ID NO: 19.

The results of this experiment are shown in FIG. 2 and Table 2. Test1 and Test2 (embodiments of the present invention) were improved over the reference plasmid. Notably, an up to 2.9-fold increase in mRNA expression was observed for Test1 plasmid over the reference plasmid, and if using sample Ref 0.0625 μg as the calibrator sample, up to 3.3-fold increase in mRNA level could be detected.

TABLE 2
Assessment of murine GTP cyclohydrolase 1 gene expression

| | Av | SD |
|---|---|---|
| Ref 0.25 μg | 1 | 0.016089 |
| Ref 0.125 μg | 0.452139 | 0.040327 |
| Ref 0.0625 μg | 0.244783 | 0.037694 |
| Test1 0.25 μg | 2.874335 | 0.029164 |
| Test1 0.125 μg | 1.309529 | 0.077492 |
| Test1 0.0625 μg | 0.8055 | 0.187179 |
| Test2 0.25 μg | 1.289959 | 0.887127 |
| Test2 0.125 μg | 1.226948 | 0.708271 |
| Test2 0.0625 μg | 0.442867 | 0.021152 |

### Example 3—In Vivo Assessment of the Construct of the Invention

The purpose of this study was to compare the ability of the inventor's construct (MRX001, FIG. 3, SEQ ID NO: 13) expressing TH and GCH1 by the same promoter with a comparator based on the prior art construct expressing the two genes by separate promoters, to improve the step count in a rat model of Parkinson's disease, using the gold standard technique of the art.

### Materials and Methods

The methods used in this example are described in detail in Cederfjall et al, Scientific

Reports, 2013; 3:2157. Therefore, this experiment represents a robust comparison with the closest prior art construct.

20× Female Sprague Dawley rats, weighing 200-250 g were used. All rats received unilateral 6-OHDA medial forebrain bundle lesions. Rats were tested for 3 and 4 week post-lesion amphetamine rotations to confirm adequate lesioning. Rats were allocated to the treatment groups to ensure that the magnitude of lesion effect was evenly distributed between the two groups. Rats received intrastriatal infusions of the reference vector (Promoter-GCH1-Promoter-TH) (n=8), the experimental vector (MRX) (n=8) or remained as lesion only controls (n=4).

Each animal in each vector group received a total of 2×10⁹ genome copies of vector in 5 ml of vehicle This was intended to be not to fully restore motor function i.e to be sufficiently high to show activity but not sufficient to achieve optimal results and leaves a clear window to demonstrate enhanced efficacy if present in the comparator. The injections were made at two sites with two 1.5 ml deposits in the ventral tract and two 1.0 ml deposits in the dorsal injection tract. The coordinates were: (1). AP: 11.0 mm; ML:−2.8 mm and DV:−4.5, −3.5 mm and (2) AP: 0.0 mm; ML:−4.0 mm and DV:−5.0, −4.0 mm with the tooth bar set to −2.4 mm. The AAV vector was injected at a speed of 0.4 ml/min and the needle was kept in place for 1 min after the ventral and 3 min after the dorsal deposit was delivered, before it was slowly retracted.

Stepping tests were performed according to the method described in Olsson et al. (Olsson, M., et al. Forelimb akinesia in the rat Parkinson model: differential effects of dopamine agonists and nigral transplants as assessed by a new stepping test. J Neurosci 15, 3863-3875 (1995). An investigator blinded to the group identities of the animals assessed forelimb use by holding the rat with two hands only allowing one forepaw to reach the table surface. The animal was then moved sideways over a defined distance at a constant speed over 4-5 sec. The investigator scored the numbers of adjusting side steps in the forehand direction twice on a testing day, and the average was calculated. The primary endpoint was prospectively defined as the difference in step count at week 4 post injection of vector.

### Results

The results of this experiment are shown in FIGS. 14 and 15. Surprisingly, the inventor has found that the claimed construct is capable of partially restoring the step count in mice models of Parkinson's disease, whereas the prior art construct provides no such effect. This result demonstrates that the inventor's construct is significantly superior, and represents an important improvement in the art.

### CONCLUSIONS

WO2013/061076 and WO2010/055209 (Oxford Biomedica) identified three proteins that were potentially effective in the treatment of Parkinson's disease, and all three proteins were regarded as being essential to provide a therapeutic effect. There is no discussion on the advantages of using a single promoter approach, as claimed, over a separate promoter approach. In fact, on page 3 in WO2013/061076, in the last line of the second paragraph, it is stated that: “ . . . increased levels of L-DOPA and dopamine were not due to increases in protein expression from the fusion design vectors . . . ”. Thus, these documents teach that, TH, GCH1 and AADC are required to produce a therapeutic effect. It is silent on the advantages or disadvantages of different promoter set-ups, as are all other relevant publications in relation to such constructs.

Cederfjall and Kirik (including WO2015/152813) moved the art forward to some extent by identifying that only two proteins were required to produce a therapeutic effect, these being TH and GCH1. However, the expression of these two genes is driven by separate promoters, and while WO2015/152813 suggests that this may be an effective means of expressing these genes for therapy, subsequently published work has proven that this approach is ineffective. The separate promoter approach surprisingly results in sub-optimal expression when translated to results outside of that of murine studies. So much so that it failed to achieve complete reversal of motor symptoms in a primate model of Parkinson's disease. As a result of this defect, the corresponding product never went into full development, and Cederfjall et al. (Scientific reports; 3:2157; 8 Jul. 2013) stated that “this problem requires a solution prior to the initiation of clinical trials utilizing this approach”.

Surprisingly, and without wishing to be bound to any particular theory, the inventor has discovered that a separate promoter approach, as used in the prior art, results in random interference between the promoters, such that the expression of one or both genes is reduced to sub-therapeutic levels. This is not described in the any prior art document in the field, and would not have been considered by the skilled person looking to solve the problem of providing an improved TH and GCH1 expression construct.

The inventor has developed a novel construct for expression of TH and GCH1 (and not AADC), wherein expression of the two genes is driven by a single promoter. This results in significantly higher levels of expression than that of the construct of WO2015/152813. For example, the in vitro data provided in FIGS. 1 and 2 compare the expression levels of TH and GCH1 with that of the control construct which places the same promoter before each gene in the same manner as described WO2015/152813. This comparative data shows that the claimed invention displays surprisingly higher levels of expression of both TH and GCH1.

The in vivo experiments performed in a murine model (FIGS. 14 and 15) confirm the in vitro findings. The methods used in the inventor's in vivo experiments are the same as those disclosed in Cederfjall et al, 2013, the authors being the inventors of WO2015/152813. Therefore, this experiment represents a robust comparison with the closest prior art construct. The control construct places the same promoter before each gene in the same manner as described, in WO2015/152813. The inventor performed the experiments at titers that are sufficiently high to show activity, but not sufficient to achieve optimal results, thus leaving a clear window to demonstrate the enhanced efficacy.

Surprisingly, the inventor has found that the claimed construct is capable of restoring the step count in mice models of Parkinson's disease, whereas construct incorporating a promoter before each gene shows no effect. Moreover, it is particularly surprising that this bicistronic construct has been demonstrated by the inventor to be markedly more effective in vitro than a 1:1 mixture of equivalent tires of a monocistronic TH vector and a monocistronic GCH1 vector.

This result demonstrates that the inventor's construct is far superior, and represents a significant improvement in the art. The inventor's construct results in improved expression of TH and GH1 and this translates to an improved therapeutic effect, as evidenced by the in vitro and in vivo data provided herein.

1-48. (canceled)

49. A genetic construct comprising:
a promoter operably linked to a first coding sequence, which encodes tyrosine hydroxylase (TH), and a second coding sequence, which encodes GTP cyclohydrolase 1 (GCH1);
wherein the first coding sequence comprises a sequence of (a) SEQ ID NO: 1 or (b) SEQ ID No:2, or a fragment or variant thereof having greater than 85% sequence identity to SEQ ID No: 1, or (c) SEQ ID No: 2, or a nucleotide sequence encoding an amino acid sequence as set out in SEQ ID NO: 21 or SEQ ID No:22, or a fragment or variant thereof having greater than 85% sequence identity to SEQ ID No: 21 or SEQ ID No: 22;
wherein the second coding sequence comprises a sequence of SEQ ID NO: 4, or a fragment or variant thereof having greater than 85% sequence identity to SEQ ID No: 4, or a nucleotide sequence encoding an amino acid sequence as set out in SEQ ID NO: 23, or a fragment or variant thereof having greater than 85% sequence identity to SEQ ID No: 23,
wherein the second coding sequence is 3′ to the first coding sequence, and the first and second coding sequences are part of a single operon,
wherein the genetic construct comprises between the first and second coding sequences an intervening sequence so as to enable, upon expression, production of a functional TH protein and a functional GCH1 protein; and
wherein the genetic construct does not encode aromatic amino acid decarboxylase (AADC).

50. The genetic construct according to claim 49, wherein the first coding sequence comprises a nucleotide sequence as set out in SEQ ID NO: 1 or SEQ ID No:2, or comprises a nucleotide sequence encoding an amino acid sequence as set out in SEQ ID NO: 21 or SEQ ID No: 22.

51. The genetic construct according to claim 49, wherein the second coding sequence comprises a nucleotide sequence as set out in SEQ ID NO: 4, or comprises a nucleotide sequence encoding an amino acid sequence as set out in SEQ ID NO: 23.

52. The genetic construct according to claim 49, wherein the intervening sequence comprises an IRES or a sequence encoding a furin cleavage site.

53. The genetic construct according to claim 49, wherein the intervening sequence comprises a nucleotide sequence as set out as SEQ ID NO: 6 or 7 or a fragment or variant thereof, or a nucleotide sequence as set out in SEQ ID No:8, or a fragment or variant thereof.

54. The genetic construct according to claim 49, wherein the promoter is a constitutive promoter, an activatable promoter, an inducible promoter, or a tissue-specific promoter.

55. The genetic construct according to claim 54, optionally wherein the promoter comprises a CMV promoter or a human synapsin promoter.

56. The genetic construct according to claim 49, wherein the promoter is operably linked to an enhancer.

57. The genetic construct according to claim 56, wherein the enhancer comprises a CMV enhancer.

58. The genetic construct according to claim 49, wherein the promoter comprises a nucleotide sequence as set out in SEQ ID No: 5 or 25.

59. The genetic construct according to claim 49, wherein the genetic construct comprises: (i) a nucleotide sequence encoding Woodchuck Hepatitis Virus Post-transcriptional Regulatory Element (WPRE), and optionally wherein the WPRE comprises a nucleic acid sequence as set out in SEQ ID NO: 10 or SEQ ID NO: 11; (ii) a nucleotide sequence encoding a polyA tail, and/or optionally wherein the poly A tail comprises a nucleic acid sequence as set out in SEQ ID No: 12; and/or (iii) left and/or right Inverted Terminal Repeat sequences (ITRs).

60. The genetic construct according to claim 49, wherein the construct comprises a sequence as set out in SEQ ID NO: 18 or SEQ ID NO: 19.

61. A recombinant vector comprising the genetic construct according to claim 49.

62. The recombinant vector according to claim 61, wherein the recombinant vector is a recombinant AAV vector.

63. The recombinant vector according to claim 61, wherein the recombinant vector comprises a sequence as set out in SEQ ID NO: 13, SEQ ID NO: 14, or SEQ ID NO: 15.

64. A method of treating, preventing, or ameliorating a neurodegenerative disorder in a subject, the method comprising administering, to a subject in need of such treatment, a therapeutically effective amount of the genetic construct according to claim 49.

65. The method of claim 64, wherein the neurodegenerative disorder is a disease associated with catecholamine dysfunction, optionally wherein the disease associated with catecholamine dysfunction is characterised by a dopamine deficiency.

66. The method of claim 64, wherein the neurodegenerative disorder is Parkinson's disease.

67. A pharmaceutical composition comprising the genetic construct according to claim 49, and a pharmaceutically acceptable vehicle.


SEQUENCE LISTING (31 sequences)

SEQ ID NO: 1 - DNA, 1494 bp, Homo sapiens
  source 1..1494: mol_type = genomic DNA
atgcccacccccgacgccaccacgccacaggccaagggcttccgcagggccgtgtctgagctggacgccaagcaggcagaggccatcatgtccccgcggttcattgggcgcaggcagagcctcatcgaggacgcccgcaaggagcgggaggcggcggtggcagcagcggccgctgcagtcccctcggagcccggggaccccctggaggctgtggcctttgaggagaaggaggggaaggccgtgctaaacctgctcttctccccgagggccaccaagccctcggcgctgtcccgagctgtgaaggtgtttgagacgtttgaagccaaaatccaccatctagagacccggcccgcccagaggccgcgagctgggggcccccacctggagtacttcgtgcgcctcgaggtgcgccgaggggacctggccgccctgctcagtggtgtgcgccaggtgtcagaggacgtgcgcagccccgcggggcccaaggtcccctggttcccaagaaaagtgtcagagctggacaagtgtcatcacctggtcaccaagttcgaccctgacctggacttggaccacccgggcttctcggaccaggtgtaccgccagcgcaggaagctgattgctgagatcgccttccagtacaggcacggcgacccgattccccgtgtggagtacaccgccgaggagattgccacctggaaggaggtctacaccacgctgaagggcctctacgccacgcacgcctgcggggagcacctggaggcctttgctttgctggagcgcttcagcggctaccgggaagacaatatcccccagctggaggacgtctcccgcttcctgaaggagcgcacgggcttccagctgcggcctgtggccggcctgctgtccgcccgggacttcctggccagcctggccttccgcgtgttccagtgcacccagtatatccgccacgcgtcctcgcccatgcactcccctgagccggactgctgccacgagctgctggggcacgtgcccatgctggccgaccgcaccttcgcgcagttctcgcaggacattggcctggcgtccctgggggcctcggatgaggaaattgagaagctgtccacgctgtactggttcacggtggagttcgggctgtgtaagcagaacggggaggtgaaggcctatggtgccgggctgctgtcctcctacggggagctcctgcactgcctgtctgaggagcctgagattcgggccttcgaccctgaggctgcggccgtgcagccctaccaagaccagacgtaccagtcagtctacttcgtgtctgagagcttcagtgacgccaaggacaagctcaggagctatgcctcacgcatccagcgccccttctccgtgaagttcgacccgtacacgctggccatcgacgtgctggacagcccccaggccgtgcggcgctccctggagggtgtccaggatgagctggacacccttgcccatgcgctgagtgccattggctag

SEQ ID NO: 2 - DNA, 1029 bp, Homo sapiens
  source 1..1029: mol_type = genomic DNA
atgagccccgcggggcccaaggtcccctggttcccaagaaaagtgtcagagctggacaagtgtcatcacctggtcaccaagttcgaccctgacctggacttggaccacccgggcttctcggaccaggtgtaccgccagcgcaggaagctgattgctgagatcgccttccagtacaggcacggcgacccgattccccgtgtggagtacaccgccgaggagattgccacctggaaggaggtctacaccacgctgaagggcctctacgccacgcacgcctgcggggagcacctggaggcctttgctttgctggagcgcttcagcggctaccgggaagacaatatcccccagctggaggacgtctcccgcttcctgaaggagcgcacgggcttccagctgcggcctgtggccggcctgctgtccgcccgggacttcctggccagcctggccttccgcgtgttccagtgcacccagtatatccgccacgcgtcctcgcccatgcactcccctgagccggactgctgccacgagctgctggggcacgtgcccatgctggccgaccgcaccttcgcgcagttctcgcaggacattggcctggcgtccctgggggcctcggatgaggaaattgagaagctgtccacgctgtactggttcacggtggagttcgggctgtgtaagcagaacggggaggtgaaggcctatggtgccgggctgctgtcctcctacggggagctcctgcactgcctgtctgaggagcctgagattcgggccttcgaccctgaggctgcggccgtgcagccctaccaagaccagacgtaccagtcagtctacttcgtgtctgagagcttcagtgacgccaaggacaagctcaggagctatgcctcacgcatccagcgccccttctccgtgaagttcgacccgtacacgctggccatcgacgtgctggacagcccccaggccgtgcggcgctccctggagggtgtccaggatgagctggacacccttgcccatgcgctgagtgccattggctag

SEQ ID NO: 3 - DNA, 792 bp, Mus musculus
  source 1..792: mol_type = genomic DNA
ggtggttttcctttgaaaaacacgatgataatatggccacaaccgcggccgtagatcccgggaccatggagaagccgcggggagtcaggtgcaccaatgggttctccgagcgggagctgccgcggcccggggccagcccgcctgccgagaagtcccggccgcccgaggccaagggcgcacagccggccgacgcctggaaggcagggcggcaccgcagcgaggaggaaaaccaggtgaacctccccaaactggcggctgcttactcgtccattctgctctcgctgggcgaggacccccagcggcaggggctgctcaagacgccctggagggcggccaccgccatgcagtacttcaccaagggataccaggagaccatctcagatgtcctgaatgatgctatatttgatgaagatcatgacgagatggtgattgtgaaggacatagatatgttctccatgtgtgagcatcaccttgttccatttgtaggaagggtccatattggctatcttcctaacaagcaagtccttggtctcagtaaacttgccaggattgtagaaatctacagtagacgactacaagttcaagagcgcctcaccaaacagattgcggtggccatcacagaagccttgcagcctgctggcgttggagtagtgattgaagcgacacacatgtgcatggtaatgcgaggcgtgcagaaaatgaacagcaagactgtcactagcaccatgctgggcgtgttccgggaagaccccaagactcgggaggagttcctcacactaatcaggagctgag

SEQ ID NO: 4 - DNA, 753 bp, Homo sapiens
  source 1..753: mol_type = genomic DNA
atggagaagggccctgtgcgggcaccggcggagaagccgcggggcgccaggtgcagcaatgggttccccgagcgggatccgccgcggcccgggcccagcaggccggcggagaagcccccgcggcccgaggccaagagcgcgcagcccgcggacggctggaagggcgagcggccccgcagcgaggaggataacgagctgaacctccctaacctggcagccgcctactcgtccatcctgagctcgctgggcgagaacccccagcggcaagggctgctcaagacgccctggagggcggcctcggccatgcagttcttcaccaagggctaccaggagaccatctcagatgtcctaaacgatgctatatttgatgaagatcatgatgagatggtgattgtgaaggacatagacatgttttccatgtgtgagcatcacttggttccatttgttggaaaggtccatattggttatcttcctaacaagcaagtccttggcctcagcaaacttgcgaggattgtagaaatctatagtagaagactacaagttcaggagcgccttacaaaacaaattgctgtagcaatcacggaagccttgcggcctgctggagtcggggtagtggttgaagcaacacacatgtgtatggtaatgcgaggtgtacagaaaatgaacagcaaaactgtgaccagcacaatgttgggtgtgttccgggaggatccaaagactcgggaagagttcctgactctcattaggagctga

SEQ ID NO: 5 - DNA, 469 bp, Homo sapiens
  source 1..469: mol_type = genomic DNA
ctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcag

SEQ ID NO: 6 - DNA, 461 bp, Foot-and-mouth disease virus
  source 1..461: mol_type = genomic DNA
agcaggtttccccaactgacacaaaacgtgcaacttgaaactccgcctggtctttccaggtctagaggggtaacactttgtactgcgtttggctccacgctcgatccactggcgagtgttagtaacagcactgttgcttcgtagcggagcatgacggccgtgggaactcctccttggtaacaaggacccacggggccaaaagccacgcccacacgggcccgtcatgtgtgcaaccccagcacggcgactttactgcgaaacccactttaaagtgacattgaaactggtacccacacactggtgacaggctaaggatgcccttcaggtaccccgaggtaacacgcgacactcgggatctgagaaggggactggggcttctataaaagcgctcggtttaaaaagcttctatgcctgaataggtgaccggaggtcggcacctttcctttgcaattactgaccac

SEQ ID NO: 7 - DNA, 551 bp, Encephalomyocarditis virus
  source 1..551: mol_type = genomic DNA
cgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctcccctcaagcgtattcaacaaggggctgaaggatgcccagaaggtaccccattgtatgggatctgatctggggcctcggtgcacatgcttttcatgtgtttagtcgaggttaaaaaacgtctaggccccccgaaccacggggacgtggttttcctttgaaaaacacgatgataata

SEQ ID NO: 8 - DNA, 84 bp, unidentified
  misc_feature 1..84: note = Viral 2A peptide spacer sequence
  source 1..84: mol_type = genomic DNA
cgcgcgaaacgcgcgccggtgaaacagaccctgaactttgatctgctgaaactggcgggcgatgtggaaagcaacccgggcccg

SEQ ID NO: 9 - DNA, 45 bp, unidentified
  misc_feature 1..45: note = Flexible linker sequence.
  source 1..45: mol_type = genomic DNA
ggaggtggcgggtccgggggcgggggtagcggtggcgggggctcc

SEQ ID NO: 10 - DNA, 592 bp, Woodchuck hepatitis virus
  source 1..592: mol_type = genomic DNA
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctg

SEQ ID NO: 11 - DNA, 247 bp, Woodchuck hepatitis virus
  source 1..247: mol_type = genomic DNA
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgt

SEQ ID NO: 12 - DNA, 224 bp, Simian virus 40
  source 1..224: mol_type = genomic DNA
agcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggta

SEQ ID NO: 13 - DNA, 6633 bp, unidentified
  misc_feature 1..6633: note = An embodiemnt of the AAAV1 vector of the invention, which includes a CMV promoter, a CMV enchancer, an EMCV IRES, and a SV40 poly A tail.
  source 1..6633: mol_type = genomic DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccggtcgcgtactagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgctgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcagatctttgtcgatcctaccatccactcgacacacccgccagcggccgctgccaagcttccgagctctcgaattcaaaggaggtacccaccatggccaccatgagccccgcggggcccaaggtcccctggttcccaagaaaagtgtcagagctggacaagtgtcatcacctggtcaccaagttcgaccctgacctggacttggaccacccgggcttctcggaccaggtgtaccgccagcgcaggaagctgattgctgagatcgccttccagtacaggcacggcgacccgattccccgtgtggagtacaccgccgaggagattgccacctggaaggaggtctacaccacgctgaagggcctctacgccacgcacgcctgcggggagcacctggaggcctttgctttgctggagcgcttcagcggctaccgggaagacaatatcccccagctggaggacgtctcccgcttcctgaaggagcgcacgggcttccagctgcggcctgtggccggcctgctgtccgcccgggacttcctggccagcctggccttccgcgtgttccagtgcacccagtatatccgccacgcgtcctcgcccatgcactcccctgagccggactgctgccacgagctgctggggcacgtgcccatgctggccgaccgcaccttcgcgcagttctcgcaggacattggcctggcgtccctgggggcctcggatgaggaaattgagaagctgtccacgctgtactggttcacggtggagttcgggctgtgtaagcagaacggggaggtgaaggcctatggtgccgggctgctgtcctcctacggggagctcctgcactgcctgtctgaggagcctgagattcgggccttcgaccctgaggctgcggccgtgcagccctaccaagaccagacgtaccagtcagtctacttcgtgtctgagagcttcagtgacgccaaggacaagctcaggagctatgcctcacgcatccagcgccccttctccgtgaagttcgacccgtacacgctggccatcgacgtgctggacagcccccaggccgtgcggcgctccctggagggtgtccaggatgagctggacacccttgcccatgcgctgagtgccattggctaaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctcccctcaagcgtattcaacaaggggctgaaggatgcccagaaggtaccccattgtatgggatctgatctggggcctcggtgcacatgcttttcatgtgtttagtcgaggttaaaaaacgtctaggccccccgaaccacggggacgtggttttcctttgaaaaacacgatgataatagccaccatggagaagggccctgtgcgggcaccggcggagaagccgcggggcgccaggtgcagcaatgggttccccgagcgggatccgccgcggcccgggcccagcaggccggcggagaagcccccgcggcccgaggccaagagcgcgcagcccgcggacggctggaagggcgagcggccccgcagcgaggaggataacgagctgaacctccctaacctggcagccgcctactcgtccatcctgagctcgctgggcgagaacccccagcggcaagggctgctcaagacgccctggagggcggcctcggccatgcagttcttcaccaagggctaccaggagaccatctcagatgtcctaaacgatgctatatttgatgaagatcatgatgagatggtgattgtgaaggacatagacatgttttccatgtgtgagcatcacttggttccatttgttggaaaggtccatattggttatcttcctaacaagcaagtccttggcctcagcaaacttgcgaggattgtagaaatctatagtagaagactacaagttcaggagcgccttacaaaacaaattgctgtagcaatcacggaagccttgcggcctgctggagtcggggtagtggttgaagcaacacacatgtgtatggtaatgcgaggtgtacagaaaatgaacagcaaaactgtgaccagcacaatgttgggtgtgttccgggaggatccaaagactcgggaagagttcctgactctcattaggagctgagccacctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccctggctgactgatacaatcgatttctggatccgcaggcctctgctagcttgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaagcttaacgcggtaaccacgtgcggacccaacggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 14 - DNA, 6542 bp, unidentified
  misc_feature 1..6542: note = An example of a AAAV1 vector of the invention. Thjis vector similar to SEQ ID NO: 13, but this particular embodiment includes an FMDV IRES instead of the EMCV IRES.
  source 1..6542: mol_type = genomic DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccggtcgcgtactagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgctgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcagatctttgtcgatcctaccatccactcgacacacccgccagcggccgctgccaagcttccgagctctcgaattcaaaggaggtacccaccatggccaccatgagccccgcggggcccaaggtcccctggttcccaagaaaagtgtcagagctggacaagtgtcatcacctggtcaccaagttcgaccctgacctggacttggaccacccgggcttctcggaccaggtgtaccgccagcgcaggaagctgattgctgagatcgccttccagtacaggcacggcgacccgattccccgtgtggagtacaccgccgaggagattgccacctggaaggaggtctacaccacgctgaagggcctctacgccacgcacgcctgcggggagcacctggaggcctttgctttgctggagcgcttcagcggctaccgggaagacaatatcccccagctggaggacgtctcccgcttcctgaaggagcgcacgggcttccagctgcggcctgtggccggcctgctgtccgcccgggacttcctggccagcctggccttccgcgtgttccagtgcacccagtatatccgccacgcgtcctcgcccatgcactcccctgagccggactgctgccacgagctgctggggcacgtgcccatgctggccgaccgcaccttcgcgcagttctcgcaggacattggcctggcgtccctgggggcctcggatgaggaaattgagaagctgtccacgctgtactggttcacggtggagttcgggctgtgtaagcagaacggggaggtgaaggcctatggtgccgggctgctgtcctcctacggggagctcctgcactgcctgtctgaggagcctgagattcgggccttcgaccctgaggctgcggccgtgcagccctaccaagaccagacgtaccagtcagtctacttcgtgtctgagagcttcagtgacgccaaggacaagctcaggagctatgcctcacgcatccagcgccccttctccgtgaagttcgacccgtacacgctggccatcgacgtgctggacagcccccaggccgtgcggcgctccctggagggtgtccaggatgagctggacacccttgcccatgcgctgagtgccattggctaaagcaggtttccccaactgacacaaaacgtgcaacttgaaactccgcctggtctttccaggtctagaggggtaacactttgtactgcgtttggctccacgctcgatccactggcgagtgttagtaacagcactgttgcttcgtagcggagcatgacggccgtgggaactcctccttggtaacaaggacccacggggccaaaagccacgcccacacgggcccgtcatgtgtgcaaccccagcacggcgactttactgcgaaacccactttaaagtgacattgaaactggtacccacacactggtgacaggctaaggatgcccttcaggtaccccgaggtaacacgcgacactcgggatctgagaaggggactggggcttctataaaagcgctcggtttaaaaagcttctatgcctgaataggtgaccggaggtcggcacctttcctttgcaattactgaccacgccaccatggagaagggccctgtgcgggcaccggcggagaagccgcggggcgccaggtgcagcaatgggttccccgagcgggatccgccgcggcccgggcccagcaggccggcggagaagcccccgcggcccgaggccaagagcgcgcagcccgcggacggctggaagggcgagcggccccgcagcgaggaggataacgagctgaacctccctaacctggcagccgcctactcgtccatcctgagctcgctgggcgagaacccccagcggcaagggctgctcaagacgccctggagggcggcctcggccatgcagttcttcaccaagggctaccaggagaccatctcagatgtcctaaacgatgctatatttgatgaagatcatgatgagatggtgattgtgaaggacatagacatgttttccatgtgtgagcatcacttggttccatttgttggaaaggtccatattggttatcttcctaacaagcaagtccttggcctcagcaaacttgcgaggattgtagaaatctatagtagaagactacaagttcaggagcgccttacaaaacaaattgctgtagcaatcacggaagccttgcggcctgctggagtcggggtagtggttgaagcaacacacatgtgtatggtaatgcgaggtgtacagaaaatgaacagcaaaactgtgaccagcacaatgttgggtgtgttccgggaggatccaaagactcgggaagagttcctgactctcattaggagctgagccacctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccctggctgactgatacaatcgatttctggatccgcaggcctctgctagcttgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaagcttaacgcggtaaccacgtgcggacccaacggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 15 - DNA, 6165 bp, unidentified
  misc_feature 1..6165: note = An example of an AAAV1 vector of the invention. This is similar to SEQ ID NO: 13, but this particular embodiment includes a Furin cleavage site and a viral 2A peptide spacer, instead of the EMCV IRES
  source 1..6165: mol_type = genomic DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccggtcgcgtactagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgctgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcagatctttgtcgatcctaccatccactcgacacacccgccagcggccgctgccaagcttccgagctctcgaattcaaaggaggtacccaccatggccaccatgagccccgcggggcccaaggtcccctggttcccaagaaaagtgtcagagctggacaagtgtcatcacctggtcaccaagttcgaccctgacctggacttggaccacccgggcttctcggaccaggtgtaccgccagcgcaggaagctgattgctgagatcgccttccagtacaggcacggcgacccgattccccgtgtggagtacaccgccgaggagattgccacctggaaggaggtctacaccacgctgaagggcctctacgccacgcacgcctgcggggagcacctggaggcctttgctttgctggagcgcttcagcggctaccgggaagacaatatcccccagctggaggacgtctcccgcttcctgaaggagcgcacgggcttccagctgcggcctgtggccggcctgctgtccgcccgggacttcctggccagcctggccttccgcgtgttccagtgcacccagtatatccgccacgcgtcctcgcccatgcactcccctgagccggactgctgccacgagctgctggggcacgtgcccatgctggccgaccgcaccttcgcgcagttctcgcaggacattggcctggcgtccctgggggcctcggatgaggaaattgagaagctgtccacgctgtactggttcacggtggagttcgggctgtgtaagcagaacggggaggtgaaggcctatggtgccgggctgctgtcctcctacggggagctcctgcactgcctgtctgaggagcctgagattcgggccttcgaccctgaggctgcggccgtgcagccctaccaagaccagacgtaccagtcagtctacttcgtgtctgagagcttcagtgacgccaaggacaagctcaggagctatgcctcacgcatccagcgccccttctccgtgaagttcgacccgtacacgctggccatcgacgtgctggacagcccccaggccgtgcggcgctccctggagggtgtccaggatgagctggacacccttgcccatgcgctgagtgccattggctaacgcgcgaaacgcgcgccggtgaaacagaccctgaactttgatctgctgaaactggcgggcgatgtggaaagcaacccgggcccggccaccatggagaagggccctgtgcgggcaccggcggagaagccgcggggcgccaggtgcagcaatgggttccccgagcgggatccgccgcggcccgggcccagcaggccggcggagaagcccccgcggcccgaggccaagagcgcgcagcccgcggacggctggaagggcgagcggccccgcagcgaggaggataacgagctgaacctccctaacctggcagccgcctactcgtccatcctgagctcgctgggcgagaacccccagcggcaagggctgctcaagacgccctggagggcggcctcggccatgcagttcttcaccaagggctaccaggagaccatctcagatgtcctaaacgatgctatatttgatgaagatcatgatgagatggtgattgtgaaggacatagacatgttttccatgtgtgagcatcacttggttccatttgttggaaaggtccatattggttatcttcctaacaagcaagtccttggcctcagcaaacttgcgaggattgtagaaatctatagtagaagactacaagttcaggagcgccttacaaaacaaattgctgtagcaatcacggaagccttgcggcctgctggagtcggggtagtggttgaagcaacacacatgtgtatggtaatgcgaggtgtacagaaaatgaacagcaaaactgtgaccagcacaatgttgggtgtgttccgggaggatccaaagactcgggaagagttcctgactctcattaggagctgagccacctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccctggctgactgatacaatcgatttctggatccgcaggcctctgctagcttgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaagcttaacgcggtaaccacgtgcggacccaacggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 16 - DNA, 6126 bp, unidentified
  misc_feature 1..6126: note = An example of an AAAV1 vector of the invention. This is similar to SEQ ID NO: 13, but this particular embodiment includes a flexible linker, instead of the EMCV IRES.
  source 1..6126: mol_type = genomic DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccggtcgcgtactagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgctgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcagatctttgtcgatcctaccatccactcgacacacccgccagcggccgctgccaagcttccgagctctcgaattcaaaggaggtacccaccatggccaccatgagccccgcggggcccaaggtcccctggttcccaagaaaagtgtcagagctggacaagtgtcatcacctggtcaccaagttcgaccctgacctggacttggaccacccgggcttctcggaccaggtgtaccgccagcgcaggaagctgattgctgagatcgccttccagtacaggcacggcgacccgattccccgtgtggagtacaccgccgaggagattgccacctggaaggaggtctacaccacgctgaagggcctctacgccacgcacgcctgcggggagcacctggaggcctttgctttgctggagcgcttcagcggctaccgggaagacaatatcccccagctggaggacgtctcccgcttcctgaaggagcgcacgggcttccagctgcggcctgtggccggcctgctgtccgcccgggacttcctggccagcctggccttccgcgtgttccagtgcacccagtatatccgccacgcgtcctcgcccatgcactcccctgagccggactgctgccacgagctgctggggcacgtgcccatgctggccgaccgcaccttcgcgcagttctcgcaggacattggcctggcgtccctgggggcctcggatgaggaaattgagaagctgtccacgctgtactggttcacggtggagttcgggctgtgtaagcagaacggggaggtgaaggcctatggtgccgggctgctgtcctcctacggggagctcctgcactgcctgtctgaggagcctgagattcgggccttcgaccctgaggctgcggccgtgcagccctaccaagaccagacgtaccagtcagtctacttcgtgtctgagagcttcagtgacgccaaggacaagctcaggagctatgcctcacgcatccagcgccccttctccgtgaagttcgacccgtacacgctggccatcgacgtgctggacagcccccaggccgtgcggcgctccctggagggtgtccaggatgagctggacacccttgcccatgcgctgagtgccattggctaaggaggtggcgggtccgggggcgggggtagcggtggcgggggctccgccaccatggagaagggccctgtgcgggcaccggcggagaagccgcggggcgccaggtgcagcaatgggttccccgagcgggatccgccgcggcccgggcccagcaggccggcggagaagcccccgcggcccgaggccaagagcgcgcagcccgcggacggctggaagggcgagcggccccgcagcgaggaggataacgagctgaacctccctaacctggcagccgcctactcgtccatcctgagctcgctgggcgagaacccccagcggcaagggctgctcaagacgccctggagggcggcctcggccatgcagttcttcaccaagggctaccaggagaccatctcagatgtcctaaacgatgctatatttgatgaagatcatgatgagatggtgattgtgaaggacatagacatgttttccatgtgtgagcatcacttggttccatttgttggaaaggtccatattggttatcttcctaacaagcaagtccttggcctcagcaaacttgcgaggattgtagaaatctatagtagaagactacaagttcaggagcgccttacaaaacaaattgctgtagcaatcacggaagccttgcggcctgctggagtcggggtagtggttgaagcaacacacatgtgtatggtaatgcgaggtgtacagaaaatgaacagcaaaactgtgaccagcacaatgttgggtgtgttccgggaggatccaaagactcgggaagagttcctgactctcattaggagctgagccacctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccctggctgactgatacaatcgatttctggatccgcaggcctctgctagcttgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaagcttaacgcggtaaccacgtgcggacccaacggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 17 - DNA, 4561 bp, unidentified
  misc_feature 1..4561: note = A vector carrying AAV2 right and left ITRs. This vector is suitable for the production of AAV vectors.
  source 1..4561: mol_type = genomic DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccggtcgcgtctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgcaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttgcaccaaaatcaacgggactttccaaaatgtcgtaacaactccgccccattgacgcaaatgggcggtaggcgtgtacggtgggaggtctatataagcagagctgtttagtgaaccgtcagatcgcctggagacgccatccacgctgttttgacctccatagaagacaccgggaccgatccagcctccgcggattcgaatcccggccgggaacggtgcattggaacgcggattccccgtgccaagagtgacgtaagtaccgcctatagagtctataggcccacaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgggattcgaacatcgattgaattcagatccgctagtaatacgactcactatagggagaggatccggtaccgaggagatctgccgccgcgatcgccggcgcgccagatctcacgcttaactagctagcggaccgacgcgtacgcggccgctcgaggattataaggatgacgacgataaattcgtcgagcaccaccaccaccaccactaataaggtttatccgatccaccggatctagataagatatccgatccaccggatctagataactgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcggtaaccacgtgcggacccaacggccgcaggaacccctagtgatggagttggccactccctctagagctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 18 - DNA, 6773 bp, unidentified
  misc_feature 1..6773: note = The genetic construct as shown in Fig. 10, which includes a CMV promoter and murine GCH1.
  source 1..6773: mol_type = genomic DNA
ggcgatcgcggctcccgacatcttggaccattagctccacaggtatcttcttccctctagtggtcataacagcagcttcagctacctctcaattcaaaaaacccctcaagacccgtttagaggccccaaggggttatgctatcaatcgttgcgttacacacacaaaaaaccaacacacatccatcttcgatggatagcgattttattatctaactgctgatcgagtgtagccagatctagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgctgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcagatctagagatcccgggaccgccaccatgagccccgcggggcccaaggtcccctggttcccaagaaaagtgtcagagctggacaagtgtcatcacctggtcaccaagttcgaccctgacctggacttggaccacccgggcttctcggaccaggtgtaccgccagcgcaggaagctgattgctgagatcgccttccagtacaggcacggcgacccgattccccgtgtggagtacaccgccgaggagattgccacctggaaggaggtctacaccacgctgaagggcctctacgccacgcacgcctgcggggagcacctggaggcctttgctttgctggagcgcttcagcggctaccgggaagacaatatcccccagctggaggacgtctcccgcttcctgaaggagcgcacgggcttccagctgcggcctgtggccggcctgctgtccgcccgggacttcctggccagcctggccttccgcgtgttccagtgcacccagtatatccgccacgcgtcctcgcccatgcactcccctgagccggactgctgccacgagctgctggggcacgtgcccatgctggccgaccgcaccttcgcgcagttctcgcaggacattggcctggcgtccctgggggcctcggatgaggaaattgagaagctgtccacgctgtactggttcacggtggagttcgggctgtgtaagcagaacggggaggtgaaggcctatggtgccgggctgctgtcctcctacggggagctcctgcactgcctgtctgaggagcctgagattcgggccttcgaccctgaggctgcggccgtgcagccctaccaagaccagacgtaccagtcagtctacttcgtgtctgagagcttcagtgacgccaaggacaagctcaggagctatgcctcacgcatccagcgccccttctccgtgaagttcgacccgtacacgctggccatcgacgtgctggacagcccccaggccgtgcggcgctccctggagggtgtccaggatgagctggacacccttgcccatgcgctgagtgccattggctaaagcaggtttccccaactgacacaaaacgtgcaacttgaaactccgcctggtctttccaggtctagaggggtaacactttgtactgcgtttggctccacgctcgatccactggcgagtgttagtaacagcactgttgcttcgtagcggagcatgacggccgtgggaactcctccttggtaacaaggacccacggggccaaaagccacgcccacacgggcccgtcatgtgtgcaaccccagcacggcgactttactgcgaaacccactttaaagtgacattgaaactggtacccacacactggtgacaggctaaggatgcccttcaggtaccccgaggtaacacgcgacactcgggatctgagaaggggactggggcttctataaaagcgctcggtttaaaaagcttctatgcctgaataggtgaccggaggtcggcacctttcctttgcaattactgaccacgccaccatggagaagccgcggggagtcaggtgcaccaatgggttctccgagcgggagctgccgcggcccggggccagcccgcctgccgagaagtcccggccgcccgaggccaagggcgcacagccggccgacgcctggaaggcagggcggcaccgcagcgaggaggaaaaccaggtgaacctccccaaactggcggctgcttactcgtccattctgctctcgctgggcgaggacccccagcggcaggggctgctcaagacgccctggagggcggccaccgccatgcagtacttcaccaagggataccaggagaccatctcagatgtcctgaatgatgctatatttgatgaagatcatgacgagatggtgattgtgaaggacatagatatgttctccatgtgtgagcatcaccttgttccatttgtaggaagggtccatattggctatcttcctaacaagcaagtccttggtctcagtaaacttgccaggattgtagaaatctacagtagacgactacaagttcaagagcgcctcaccaaacagattgcggtggccatcacagaagccttgcagcctgctggcgttggagtagtgattgaagcgacacacatgtgcatggtaatgcgaggcgtgcagaaaatgaacagcaagactgtcactagcaccatgctgggcgtgttccgggaagaccccaagactcgggaggagttcctcacactaatcaggagctgaggccacctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttcccatatgcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtattggcccatctctatcggtatcgtagcataaccccttggggcctctaaacgggtcttgaggggttttttgtgcccctcgggccggattgctatctaccggcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtcagctatcctgcaggcgatctctcgatttcgatcaagacattcctttaatggtcttttctggacaccactaggggtcagaagtagttcatcaaactttcttccctccctaatctcattggttaccttgggctatcgaaacttaattaaccagtcaagtcagctacttggcgagatcgacttgtctgggtttcgactacgctcagaattgcgtcagtcaagttcgatctggtccttgctattgcacccgttctccgattacgagtttcatttaaatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcatttaaatttccgaactctccaaggccctcgtcggaaaatcttcaaacctttcgtccgatccatcttgcaggctacctctcgaacgaactatcgcaagtctcttggccggccttgcgccttggctattgcttggcagcgcctatcgccaggtattactccaatcccgaatatccgagatcgggatcacccgagagaagttcaacctacatcctcaatcccgatctatccgagatccgaggaatatcgaaatcggggcgcgcctggtgtaccgagaacgatcctctcagtgcgagtctcgacgatccatatcgttgcttggcagtcagccagtcggaatccagcttgggacccaggaagtccaatcgtcagatattgtactcaagcctggtcacggcagcgtaccgatctgtttaaacctagatattgatagtctgatcggtcaacgtataatcgagtcctagcttttgcaaacatctatcaagagacaggatcagcaggaggctttcgcatgagtattcaacatttccgtgtcgcccttattcccttttttgcggcattttgccttcctgtttttgctcacccagaaacgctggtgaaagtaaaagatgctgaagatcagttgggtgcgcgagtgggttacatcgaactggatctcaacagcggtaagatccttgagagttttcgccccgaagaacgctttccaatgatgagcacttttaaagttctgctatgtggcgcggtattatcccgtattgacgccgggcaagagcaactcggtcgccgcatacactattctcagaatgacttggttgagtattcaccagtcacagaaaagcatcttacggatggcatgacagtaagagaattatgcagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaaccttgcgtaaactattaactggcgaactacttactctagcttcccggcaacagttgatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaaccgattctaggtgcattggcgcagaaaaaaatgcctgatgcgacgctgcgcgtcttatactcccacatatgccagattcagcaacggatacggcttccccaacttgcccacttccatacgtgtcctccttaccagaaatttatccttaagatcccgaatcgtttaaactcgactctggctctatcgaatctccgtcgtttcgagcttacgcgaacagccgtggcgctcatttgctcgtcgggcatcgaatctcgtcagctatcgtcagcttacctttttggca

SEQ ID NO: 19 - DNA, 6390 bp, unidentified
  misc_feature 1..6390: note = The genetic construct as shown in Fig. 11. This embodiment includes a CMV promoter and murine GCH1.
  source 1..6390: mol_type = genomic DNA
ggcgatcgcggctcccgacatcttggaccattagctccacaggtatcttcttccctctagtggtcataacagcagcttcagctacctctcaattcaaaaaacccctcaagacccgtttagaggccccaaggggttatgctatcaatcgttgcgttacacacacaaaaaaccaacacacatccatcttcgatggatagcgattttattatctaactgctgatcgagtgtagccagatctagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgctgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcagatctagagatcccgggaccgccaccatgagccccgcggggcccaaggtcccctggttcccaagaaaagtgtcagagctggacaagtgtcatcacctggtcaccaagttcgaccctgacctggacttggaccacccgggcttctcggaccaggtgtaccgccagcgcaggaagctgattgctgagatcgccttccagtacaggcacggcgacccgattccccgtgtggagtacaccgccgaggagattgccacctggaaggaggtctacaccacgctgaagggcctctacgccacgcacgcctgcggggagcacctggaggcctttgctttgctggagcgcttcagcggctaccgggaagacaatatcccccagctggaggacgtctcccgcttcctgaaggagcgcacgggcttccagctgcggcctgtggccggcctgctgtccgcccgggacttcctggccagcctggccttccgcgtgttccagtgcacccagtatatccgccacgcgtcctcgcccatgcactcccctgagccggactgctgccacgagctgctggggcacgtgcccatgctggccgaccgcaccttcgcgcagttctcgcaggacattggcctggcgtccctgggggcctcggatgaggaaattgagaagctgtccacgctgtactggttcacggtggagttcgggctgtgtaagcagaacggggaggtgaaggcctatggtgccgggctgctgtcctcctacggggagctcctgcactgcctgtctgaggagcctgagattcgggccttcgaccctgaggctgcggccgtgcagccctaccaagaccagacgtaccagtcagtctacttcgtgtctgagagcttcagtgacgccaaggacaagctcaggagctatgcctcacgcatccagcgccccttctccgtgaagttcgacccgtacacgctggccatcgacgtgctggacagcccccaggccgtgcggcgctccctggagggtgtccaggatgagctggacacccttgcccatgcgctgagtgccattggctaacgcgcgaaacgcgcgccggtgaaacagaccctgaactttgatctgctgaaactggcgggcgatgtggaaagcaacccgggcccgatggagaagccgcggggagtcaggtgcaccaatgggttctccgagcgggagctgccgcggcccggggccagcccgcctgccgagaagtcccggccgcccgaggccaagggcgcacagccggccgacgcctggaaggcagggcggcaccgcagcgaggaggaaaaccaggtgaacctccccaaactggcggctgcttactcgtccattctgctctcgctgggcgaggacccccagcggcaggggctgctcaagacgccctggagggcggccaccgccatgcagtacttcaccaagggataccaggagaccatctcagatgtcctgaatgatgctatatttgatgaagatcatgacgagatggtgattgtgaaggacatagatatgttctccatgtgtgagcatcaccttgttccatttgtaggaagggtccatattggctatcttcctaacaagcaagtccttggtctcagtaaacttgccaggattgtagaaatctacagtagacgactacaagttcaagagcgcctcaccaaacagattgcggtggccatcacagaagccttgcagcctgctggcgttggagtagtgattgaagcgacacacatgtgcatggtaatgcgaggcgtgcagaaaatgaacagcaagactgtcactagcaccatgctgggcgtgttccgggaagaccccaagactcgggaggagttcctcacactaatcaggagctgaggccacctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttcccatatgcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtattggcccatctctatcggtatcgtagcataaccccttggggcctctaaacgggtcttgaggggttttttgtgcccctcgggccggattgctatctaccggcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtcagctatcctgcaggcgatctctcgatttcgatcaagacattcctttaatggtcttttctggacaccactaggggtcagaagtagttcatcaaactttcttccctccctaatctcattggttaccttgggctatcgaaacttaattaaccagtcaagtcagctacttggcgagatcgacttgtctgggtttcgactacgctcagaattgcgtcagtcaagttcgatctggtccttgctattgcacccgttctccgattacgagtttcatttaaatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcatttaaatttccgaactctccaaggccctcgtcggaaaatcttcaaacctttcgtccgatccatcttgcaggctacctctcgaacgaactatcgcaagtctcttggccggccttgcgccttggctattgcttggcagcgcctatcgccaggtattactccaatcccgaatatccgagatcgggatcacccgagagaagttcaacctacatcctcaatcccgatctatccgagatccgaggaatatcgaaatcggggcgcgcctggtgtaccgagaacgatcctctcagtgcgagtctcgacgatccatatcgttgcttggcagtcagccagtcggaatccagcttgggacccaggaagtccaatcgtcagatattgtactcaagcctggtcacggcagcgtaccgatctgtttaaacctagatattgatagtctgatcggtcaacgtataatcgagtcctagcttttgcaaacatctatcaagagacaggatcagcaggaggctttcgcatgagtattcaacatttccgtgtcgcccttattcccttttttgcggcattttgccttcctgtttttgctcacccagaaacgctggtgaaagtaaaagatgctgaagatcagttgggtgcgcgagtgggttacatcgaactggatctcaacagcggtaagatccttgagagttttcgccccgaagaacgctttccaatgatgagcacttttaaagttctgctatgtggcgcggtattatcccgtattgacgccgggcaagagcaactcggtcgccgcatacactattctcagaatgacttggttgagtattcaccagtcacagaaaagcatcttacggatggcatgacagtaagagaattatgcagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaaccttgcgtaaactattaactggcgaactacttactctagcttcccggcaacagttgatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaaccgattctaggtgcattggcgcagaaaaaaatgcctgatgcgacgctgcgcgtcttatactcccacatatgccagattcagcaacggatacggcttccccaacttgcccacttccatacgtgtcctccttaccagaaatttatccttaagatcccgaatcgtttaaactcgactctggctctatcgaatctccgtcgtttcgagcttacgcgaacagccgtggcgctcatttgctcgtcgggcatcgaatctcgtcagctatcgtcagcttacctttttggca

SEQ ID NO: 20 - DNA, 6380 bp, unidentified
  misc_feature 1..6380: note = The genetic construct is shown in Fig. 12. This embodiment includes a CMV promoter and murine GCH1.
  source 1..6380: mol_type = genomic DNA
ggcgatcgcggctcccgacatcttggaccattagctccacaggtatcttcttccctctagtggtcataacagcagcttcagctacctctcaattcaaaaaacccctcaagacccgtttagaggccccaaggggttatgctatcaatcgttgcgttacacacacaaaaaaccaacacacatccatcttcgatggatagcgattttattatctaactgctgatcgagtgtagccagatctagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgctgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcagatctagagatcccgggaccgccaccatgagccccgcggggcccaaggtcccctggttcccaagaaaagtgtcagagctggacaagtgtcatcacctggtcaccaagttcgaccctgacctggacttggaccacccgggcttctcggaccaggtgtaccgccagcgcaggaagctgattgctgagatcgccttccagtacaggcacggcgacccgattccccgtgtggagtacaccgccgaggagattgccacctggaaggaggtctacaccacgctgaagggcctctacgccacgcacgcctgcggggagcacctggaggcctttgctttgctggagcgcttcagcggctaccgggaagacaatatcccccagctggaggacgtctcccgcttcctgaaggagcgcacgggcttccagctgcggcctgtggccggcctgctgtccgcccgggacttcctggccagcctggccttccgcgtgttccagtgcacccagtatatccgccacgcgtcctcgcccatgcactcccctgagccggactgctgccacgagctgctggggcacgtgcccatgctggccgaccgcaccttcgcgcagttctcgcaggacattggcctggcgtccctgggggcctcggatgaggaaattgagaagctgtccacgctgtactggttcacggtggagttcgggctgtgtaagcagaacggggaggtgaaggcctatggtgccgggctgctgtcctcctacggggagctcctgcactgcctgtctgaggagcctgagattcgggccttcgaccctgaggctgcggccgtgcagccctaccaagaccagacgtaccagtcagtctacttcgtgtctgagagcttcagtgacgccaaggacaagctcaggagctatgcctcacgcatccagcgccccttctccgtgaagttcgacccgtacacgctggccatcgacgtgctggacagcccccaggccgtgcggcgctccctggagggtgtccaggatgagctggacacccttgcccatgcgctgagtgccattggcggaggtggcgggtccgggggcgggggtagcggtggcgggggctccgccaccatggagaagggccctgtgcgggcaccggcggagaagccgcggggcgccaggtgcagcaatgggttccccgagcgggatccgccgcggcccgggcccagcaggccggcggagaagcccccgcggcccgaggccaagagcgcgcagcccgcggacggctggaagggcgagcggccccgcagcgaggaggataacgagctgaacctccctaacctggcagccgcctactcgtccatcctgagctcgctgggcgagaacccccagcggcaagggctgctcaagacgccctggagggcggcctcggccatgcagttcttcaccaagggctaccaggagaccatctcagatgtcctaaacgatgctatatttgatgaagatcatgatgagatggtgattgtgaaggacatagacatgttttccatgtgtgagcatcacttggttccatttgttggaaaggtccatattggttatcttcctaacaagcaagtccttggcctcagcaaacttgcgaggattgtagaaatctatagtagaagactacaagttcaggagcgccttacaaaacaaattgctgtagcaatcacggaagccttgcggcctgctggagtcggggtagtggttgaagcaacacacatgtgtatggtaatgcgaggtgtacagaaaatgaacagcaaaactgtgaccagcacaatgttgggtgtgttccgggaggatccaaagactcgggaagagttcctgactctcattaggagctgagccacctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttcccatatgcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtattggcccatctctatcggtatcgtagcataaccccttggggcctctaaacgggtcttgaggggttttttgtgcccctcgggccggattgctatctaccggcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtcagctatcctgcaggcgatctctcgatttcgatcaagacattcctttaatggtcttttctggacaccactaggggtcagaagtagttcatcaaactttcttccctccctaatctcattggttaccttgggctatcgaaacttaattaaccagtcaagtcagctacttggcgagatcgacttgtctgggtttcgactacgctcagaattgcgtcagtcaagttcgatctggtccttgctattgcacccgttctccgattacgagtttcatttaaatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcatttaaatttccgaactctccaaggccctcgtcggaaaatcttcaaacctttcgtccgatccatcttgcaggctacctctcgaacgaactatcgcaagtctcttggccggccttgcgccttggctattgcttggcagcgcctatcgccaggtattactccaatcccgaatatccgagatcgggatcacccgagagaagttcaacctacatcctcaatcccgatctatccgagatccgaggaatatcgaaatcggggcgcgcctggtgtaccgagaacgatcctctcagtgcgagtctcgacgatccatatcgttgcttggcagtcagccagtcggaatccagcttgggacccaggaagtccaatcgtcagatattgtactcaagcctggtcacggcagcgtaccgatctgtttaaacctagatattgatagtctgatcggtcaacgtataatcgagtcctagcttttgcaaacatctatcaagagacaggatcagcaggaggctttcgcatgagtattcaacatttccgtgtcgcccttattcccttttttgcggcattttgccttcctgtttttgctcacccagaaacgctggtgaaagtaaaagatgctgaagatcagttgggtgcgcgagtgggttacatcgaactggatctcaacagcggtaagatccttgagagttttcgccccgaagaacgctttccaatgatgagcacttttaaagttctgctatgtggcgcggtattatcccgtattgacgccgggcaagagcaactcggtcgccgcatacactattctcagaatgacttggttgagtattcaccagtcacagaaaagcatcttacggatggcatgacagtaagagaattatgcagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaaccttgcgtaaactattaactggcgaactacttactctagcttcccggcaacagttgatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaaccgattctaggtgcattggcgcagaaaaaaatgcctgatgcgacgctgcgcgtcttatactcccacatatgccagattcagcaacggatacggcttccccaacttgcccacttccatacgtgtcctccttaccagaaatttatccttaagatcccgaatcgtttaaactcgactctggctctatcgaatctccgtcgtttcgagcttacgcgaacagccgtggcgctcatttgctcgtcgggcatcgaatctcgtcagctatcgtcagcttacctttttggca

SEQ ID NO: 21 - protein, 497 aa, Homo sapiens
MPTPDATTPQAKGFRRAVSELDAKQAEAIMSPRFIGRRQSLIEDARKEREAAVAAAAAAVPSEPGDPLEAVAFEEKEGKAVLNLLFSPRATKPSALSRAVKVFETFEAKIHHLETRPAQRPRAGGPHLEYFVRLEVRRGDLAALLSGVRQVSEDVRSPAGPKVPWFPRKVSELDKCHHLVTKFDPDLDLDHPGFSDQVYRQRRKLIAEIAFQYRHGDPIPRVEYTAEEIATWKEVYTTLKGLYATHACGEHLEAFALLERFSGYREDNIPQLEDVSRFLKERTGFQLRPVAGLLSARDFLASLAFRVFQCTQYIRHASSPMHSPEPDCCHELLGHVPMLADRTFAQFSQDIGLASLGASDEEIEKLSTLYWFTVEFGLCKQNGEVKAYGAGLLSSYGELLHCLSEEPEIRAFDPEAAAVQPYQDQTYQSVYFVSESFSDAKDKLRSYASRIQRPFSVKFDPYTLAIDVLDSPQAVRRSLEGVQDELDTLAHALSAIG

SEQ ID NO: 22 - protein, 342 aa, Homo sapiens
MSPAGPKVPWFPRKVSELDKCHHLVTKFDPDLDLDHPGFSDQVYRQRRKLIAEIAFQYRHGDPIPRVEYTAEEIATWKEVYTTLKGLYATHACGEHLEAFALLERFSGYREDNIPQLEDVSRFLKERTGFQLRPVAGLLSARDFLASLAFRVFQCTQYIRHASSPMHSPEPDCCHELLGHVPMLADRTFAQFSQDIGLASLGASDEEIEKLSTLYWFTVEFGLCKQNGEVKAYGAGLLSSYGELLHCLSEEPEIRAFDPEAAAVQPYQDQTYQSVYFVSESFSDAKDKLRSYASRIQRPFSVKFDPYTLAIDVLDSPQAVRRSLEGVQDELDTLAHALSAIG

SEQ ID NO: 23 - protein, 250 aa, Homo sapiens
MEKGPVRAPAEKPRGARCSNGFPERDPPRPGPSRPAEKPPRPEAKSAQPADGWKGERPRSEEDNELNLPNLAAAYSSILSSLGENPQRQGLLKTPWRAASAMQFFTKGYQETISDVLNDAIFDEDHDEMVIVKDIDMFSMCEHHLVPFVGKVHIGYLPNKQVLGLSKLARIVEIYSRRLQVQERLTKQIAVAITEALRPAGVGVVVEATHMCMVMRGVQKMNSKTVTSTMLGVFREDPKTREEFLTLIRS

SEQ ID NO: 24 - protein, 15 aa, unidentified
  REGION 1..15: note = Flexible linker sequence.
GGGGSGGGGSGGGGS

SEQ ID NO: 25 - DNA, 663 bp, unidentified
  misc_feature 1..663: note = CMV promoter
  source 1..663: mol_type = genomic DNA
acgcgtggagctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgtcaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttgcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcc

SEQ ID NO: 26 - DNA, 1338 bp, unidentified
  misc_feature 1..1338: note = OIPR sequence
  source 1..1338: mol_type = genomic DNA
attgggatcttcacacagcaggtaaggttgcgggccgggcctgggccgggtccgggccgggtattgcccgcctaatgagcgggcttttttttcttaccccttcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcctgcaaaccacgttgtggtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaggtaccccggcccgcactgacccctggtgttgctttttttttttaggccgcaagctgaagcgtgtcc

SEQ ID NO: 27 - DNA, 60 bp, unidentified
  misc_feature 1..60: note = E2A sequence
  source 1..60: mol_type = genomic DNA
cagtgtactaattatgctctcttgaaattggctggagatgttgagagcaaccctggacct

SEQ ID NO: 28 - DNA, 66 bp, unidentified
  misc_feature 1..66: note = F2A sequence.
  source 1..66: mol_type = genomic DNA
gtgaaacagactttgaattttgaccttctcaagttggcgggagacgtggagtccaaccctggacct

SEQ ID NO: 29 - DNA, 57 bp, unidentified
  misc_feature 1..57: note = P2A sequence.
  source 1..57: mol_type = genomic DNA
gccacgaacttctctctgttaaagcaagcaggagatgttgaagaaaaccccgggcct

SEQ ID NO: 30 - DNA, 54 bp, unidentified
  misc_feature 1..54: note = T2A sequence.
  source 1..54: mol_type = genomic DNA
gagggcaggggaagtcttctaacatgcggggacgtggaggaaaatcccggcccc

SEQ ID NO: 31 - skipped